(19) United States
(12) Patent Application Publication
(10) Pub. No.: US 2007/0021419 A1
(43) Pub. Date: Jan. 25, 2007

(54) 2-Aminophenyl-4-phenylpyrimidines as kinase inhibitors

(75) Inventors: Shudong Wang, Nottingham (GB); Janice McLachlan, Dundee (GB); Darren Gibson, Dundee (GB); Ashley Causton, Kent (GB); Nicholas John Turner, Edinburgh (GB); Peter Martin Fischer, Nottingham (GB)

Correspondence Address: LAHIVE & COCKFIELD, LLP, ONE POST OFFICE SQUARE, BOSTON, MA, 02109-2127, US

(73) Assignee: Cyclacel Limited, Dundee (GB)

(21) Appl. No.: 11/339,058

(22) Filed: Jan. 25, 2006

Related U.S. Application Data
(63) Continuation of application No. PCT/GB04/03284, filed on Jul. 30, 2004.

(30) Foreign Application Priority Data
Aug. 5, 2003 (GB) 0318345.6
Jul. 30, 2003 (GB) 0317841.5

(51) Int. Cl. A61K 31/5377; A61K 31/506; C07D 413/14; C07D 403/14

(52) U.S. Cl. 514/235.5; 544/122; 544/295; 544/329; 544/330; 514/252.14; 514/252.18; 514/275

(57) ABSTRACT

The present invention relates to compounds of formula I, or pharmaceutically acceptable salt thereof, 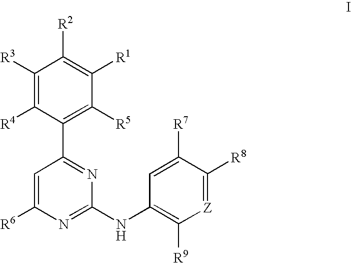 wherein:
Z is CR¹⁰ or N;
one of R¹ and R² is selected from (CH₂)ₘR¹¹, (CH₂)ₘR¹², (CH₂)ₘNR¹²R¹³,
(CH₂)ₘOR¹², (CH₂)ₘNR¹³CO(CH₂)ₙR¹¹, (CH₂)ₘNR¹³COR¹²,
(CH₂)ₘCONR¹³(CH₂)ₙR¹¹, (CH₂)ₘCONR¹²R¹³, (CH₂)ₘCO(CH₂)ₙR¹¹ and
(CH₂)ₘCOR¹²; where m is 0, 1, 2, 3 or 4 and n is 1, 2, 3 or 4; the other of R¹ and R² is H or R¹¹;
R³ and R⁵ are both H; R⁴ is H or R¹¹;
R⁶is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, (CH₂)_{q}OR¹³(CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹², (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group; providing that when
   Z is CR¹⁰ and R⁹ is H, at least one of R⁷, R⁸ and R¹⁰ is other than OMe; and
   Z is CR¹⁰ and R⁷⁻⁹ are all H, R¹⁰ is other than OCF₂CHF₂.

### RELATED APPLICATIONS

This application is a continuation of PCT/GB2004/003284, filed on Jul. 30, 2004, which claims priority to GB 0318345.6, filed on Aug. 5, 2003 and GB 0317841.5, filed on Jul. 30, 2003. The entire contents of each of these applications are hereby incorporated herein by reference.

### BACKGROUND TO THE INVENTION

In eukaryotes, all biological functions, including DNA replication, cell cycle progression, energy metabolism, and cell growth and differentiation, are regulated through the reversible phosphorylation of proteins. The phosphorylation state of a protein determines not only its function, subcellular distribution, and stability, but also what other proteins or cellular components it associates with. The balance of specific phosphorylation in the proteome as a whole, as well as of individual members in a biochemical pathway, is thus used by organisms as a strategy to maintain homeostasis in response to an ever-changing environment. The enzymes that carry out these phosphorylation and dephosphorylation steps are protein kinases and phosphatases, respectively.

The eukaryotic protein kinase family is one of the largest in the human genome, comprising some 500 genes [1,2]. The majority of kinases contain a 250-300 amino acid residue catalytic domain with a conserved core structure. This domain comprises a binding pocket for ATP (less frequently GTP), whose terminal phosphate group the kinase transfers covalently to its macromolecular substrates. The phosphate donor is always bound as a complex with a divalent ion (usually Mg²⁺ or Mn²⁺). Another important function of the catalytic domain is the binding and orientation for phosphotransfer of the macromolecular substrate. The catalytic domains present in most kinases are more or less homologous.

A wide variety of molecules capable of inhibiting protein kinase function through antagonising ATP binding are known in the art [3-7]. By way of example, the applicant has previously disclosed 2-anilino-4-heteroaryl-pyrimidine compounds with kinase inhibitory properties, particularly against cyclin-dependent kinases (CDKS) [8-12]. CDKs are serine/threonine protein kinases that associate with various cyclin subunits. These complexes are important for the regulation of eukaryotic cell cycle progression, but also for the regulation of transcription [13,14].

The present invention seeks to provide [4-(3-substituted-phenyl)-pyrimidin-2-yl]-phenyl-amines and [4-(3-substituted-phenyl)-pyrimidin-2-yl]-(pyridine-3-yl)-amines. More specifically, the invention relates to compounds that have broad therapeutic applications in the treatment of a number of different diseases and/or that are capable of lo inhibiting one or more protein kinases.

### STATEMENT OF INVENTION

The present invention relates to substituted pyrimidine derivatives. In particular, the invention relates to [4-(3-substituted-phenyl)-pyrimidin-2-yl]-phenyl-amines and [4-(3-substituted-phenyl)-pyrimidin-2-yl]-(pyridine-3-yl)-amines and their use in therapy. More specifically, but not exclusively, the invention relates to compounds that are capable of inhibiting one or more protein kinases.

A first aspect of the invention relates to compounds of formula I, or pharmaceutically acceptable salts thereof, 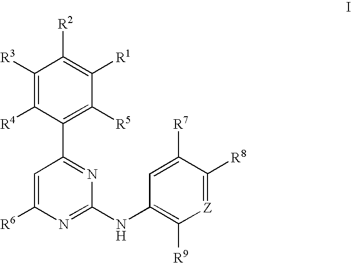
wherein:
Z is CR¹⁰ or N;
one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)ₙR¹¹, (CH₂)ₘCONR¹²R¹³, (CH₂)ₘCO(CH₂)ₙR¹¹ and (CH₂)ₘCOR¹²; where m is 0, 1, 2, 3 or 4 and n is 1, 2, 3 or 4;
the other of R¹ and R² is H or R¹¹;
R³ and R⁵ are both H;
R⁴ is H or R¹¹;
R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R⁴, OCH₂CH₂OH, OCH₂CH₂OMe, morpholino, piperidinyl, and piperazinyl;
each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹², (CH₂)ₛNR¹²R¹³, morpholino, piperidinyl or piperazinyl, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
providing that when
   Z is CR¹⁰ and R⁹ is H, at least one of R⁷, R⁸ and R¹⁰ is other than OMe; and
   Z is CR¹⁰ and R⁷⁻⁹ are all H, R¹⁰ is other than OCF₂CHF₂.

A second aspect of the invention relates to a pharmaceutical composition comprising a compound of formula I as defined above admixed with a pharmaceutically acceptable diluent, excipient or carrier.

Further aspects of the invention relate to the use of compounds of formula I as defined above in the preparation of a medicament for treating one or more of the following:
a proliferative disorder;
a viral disorder;
a CNS disorder;
a stroke;
alopecia; and
diabetes.

Another aspect of the invention relates to the use of compounds of formula I as defined above in an assay for identifying further candidate compounds capable of inhibiting one or more of a cyclin dependent kinase, GSK, aurora kinase and a PLK enzyme.

### DETAILED DESCRIPTION

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, alicyclic, heteroalicyclic or alkenyl group. More preferably, the hydrocarbyl group is an aryl, heteroaryl, alkyl, cycloalkyl, aralkyl or alkenyl group. The hydrocarbyl group may be optionally substituted by one or more R¹¹ groups.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more R¹¹ groups. Preferably, the alkyl group is unsubstituted.

As used herein, the term “cycloalkyl” refers to a cyclic alkyl group which may be substituted (mono- or poly-) or unsubstituted. Preferably, the cycloalkyl group is a C₃₋₁₂ cycloalkyl group. Suitable substituents include, for example, one or more R¹¹ groups.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group. Suitable substituents include, for example, one or more R¹¹ groups as defined above.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R¹¹ groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Again, suitable substituents include, for example, one or more R¹¹ groups.

As used herein, the term “alicyclic” refers to a cyclic aliphatic group which optionally contains one or more heteroatoms. Preferred alicyclic groups include piperidinyl, pyrrolidinyl, piperazinyl and morpholino. More preferably, the alicyclic group is selected from N-piperidinyl, N-pyrrolidinyl, N-piperazinyl and N-morpholino

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

One preferred embodiment of the invention relates to compounds of formula Ia, or pharmaceutically acceptable salts thereof, 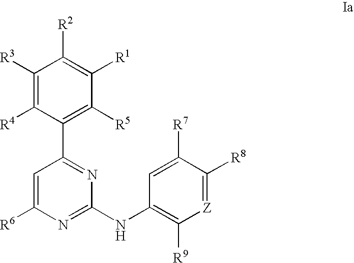
wherein:
Z is CR¹⁰ or N;
R¹ is selected from (CH₂)ₘR¹¹, (CH₂)ₘR¹², (CH₂)ₘNR¹²R¹³, (CH₂)ₘOR¹², (CH₂)ₘNR¹³CO(CH₂)R¹¹, (CH₂)ₘNR¹³COR¹², (CH₂)ₘCONR¹³(CH₂)ₙR¹¹, (CH₂)ₘCONR¹²R¹³, (CH₂)ₘCO(CH₂)ₙR¹¹ and (CH₂)ₘCOR¹²; where m is 0, 1, 2, 3 or 4 and n is 1, 2, 3 or 4;
R³ and R⁵ are both H;
R² and R⁴ are each independently H or R¹¹;
R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³ NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, OR¹³, NR¹³R¹⁴, NHCOR¹³, CF₃, COR¹³, R¹³, CONR¹³R¹⁴, SO₂NR¹³R¹⁴, SO₂R¹³, OR¹³, NR¹³SO₂R¹⁴, OCH₂CH₂OH, OCH₂CH₂OMe, morpholine, piperidine or piperazine;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
providing that when
   Z is CR¹⁰ and R⁹ is H, at least one of R⁷, R⁸ and R⁹ is other than OMe; and
   Z is CR¹⁰ and R⁷⁻⁹ are all H, R¹⁰ is other than OCF₂CHF₂.

In one preferred embodiment of the invention, one of R¹ and R² is selected from (CH₂)ₘR¹¹, (CH₂)ₘR¹², (CH₂)ₘNR¹²R¹³, (CH₂)ₘNR¹³COR¹², and (CH₂)ₘOR².

In one preferred embodiment of the invention, R¹ is selected from (CH₂)ₘR¹¹, (CH₂)ₘR¹², (CH₂)ₘNR¹²R¹³, (CH₂)ₘNR¹³COR¹³, and (CH₂)ₘOR¹².

In one preferred embodiment, one of R¹ and R² is selected from NO₂, CN, halogen, CH₂R¹¹, CH₂R¹², OR¹², NR¹²R¹³, NR¹³COR¹², CH₂NR¹²R¹³, CH₂NHSO₂R¹⁴, CF₃, NR¹³R¹⁴, R¹³, CH₂NR¹³COR¹² and NR¹³SO₂R¹².

In another preferred embodiment, R¹ is selected from NO₂, CN, halogen, CH₂R¹¹, CH₂R¹², OR¹², NR¹² R¹³, NR¹³COR¹², CH₂NR¹²R¹³, CH₂NHSO₂R¹⁴, CF₃, NR¹³R¹⁴, R¹³, CH₂NR¹³COR¹² and NR¹³SO₂R¹².

In one particularly preferred embodiment of the invention, R¹ is selected from NO₂, CN, halogen, (CH₂)ₘR¹¹, (CH₂)ₘR¹², (CH₂)ₘNR¹²R¹³, (CH₂)ₘNR¹³COR¹², and (CH₂)ₘOR¹².

In another preferred embodiment, R¹ is selected from NO₂, CN, halogen, CH₂R¹¹, CH₂R¹², OR¹², NR¹²R¹³ NR¹³COR¹², CH₂NR¹²R¹³ and CH₂NHSO₂R¹⁴.

In one preferred embodiment, R⁴ is H, OR¹³, halogen or R¹³.

In a more preferred embodiment, R⁴ is H, OMe, Me or F.

In one particularly preferred embodiment, each R¹² is independently selected from alkyl, alkenyl, alkynyl, aralkyl, a cyclic group, a saturated or unsaturated alicyclic group, and an aryl group, each of which may optionally contain one to four heteroatoms selected from O, S, and N, and each of which may optionally be substituted with one, two or three R¹¹ groups.

In one particularly preferred embodiment, each R¹² is independently selected from alkyl, alkenyl, alkynyl, aralkyl, a heteroaryl group, a saturated or unsaturated alicyclic group optionally contain one to four heteroatoms selected from O, S, and N, and an aryl group, each of which may optionally be substituted with one, two or three R¹¹ groups. In one preferred embodiment, R¹² is selected from aryl, aralkyl heteroaryl and a saturated alicyclic group optionally contain one to four heteroatoms selected from O, S, and N, each of which may optionally be substituted with one, two or three R¹¹ groups.

In a more preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three R¹¹ groups.

In an even more preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three substituents selected from NO₂, CONR¹³ R¹⁴, (CH₂)_{q}OR¹³ and R¹³.

In a further preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three substituents selected from NO₂, CONH₂, CH₂CH₂OH, CH₂OH and Me groups.

Preferably, R¹⁵ is a C₁₋₅ alkyl group.

Preferably, each R¹³ and each R¹⁴ is independently H or a C₁₋₅ alkyl group.

Even more preferably, each R¹³ and R¹⁴ is independently H or an unsubstituted C₁₋₅ alkyl group.

In one especially preferred embodiment of the invention,
each R¹² is independently selected from alkyl, alkenyl, alkynyl, aralkyl, a cyclic group, a saturated or unsaturated alicyclic group, and an aryl group, each of which may optionally contain one to four heteroatoms selected from O, S, and N, and each of which may optionally be substituted with one, two or three R¹¹ groups;
each R¹³ and each R¹⁴ is independently H or a C₁₋₅ alkyl group; and
R¹⁵ is a C₁₋₅ alkyl group.

Preferably, R¹⁵ is an unsubstituted C₁₋₅ alkyl group.

In one preferred embodiment, each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, NR¹³SO₂R¹², (CH₂)ₛNR¹²R¹³, morpholino, piperidinyl or piperazinyl, where q, r and s are each independently 0, 1, 2, 3 or 4.

In another preferred embodiment, each R¹¹ is selected from halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

In another especially preferred embodiment, R¹¹ is selected from halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

In a preferred embodiment, one of R¹ and R² is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂

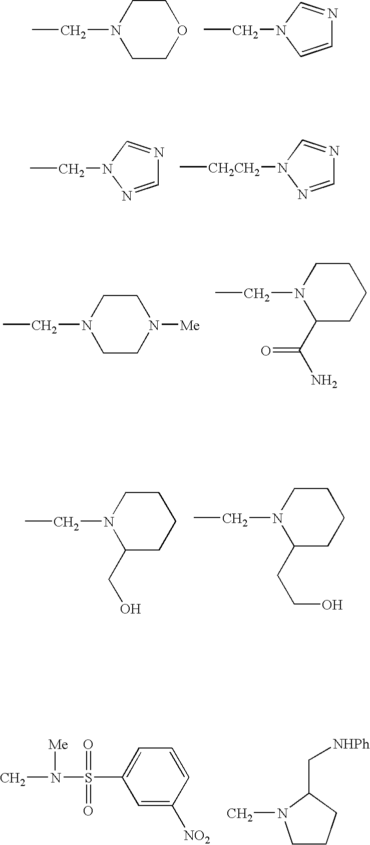

In a more preferred embodiment, R¹ is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂

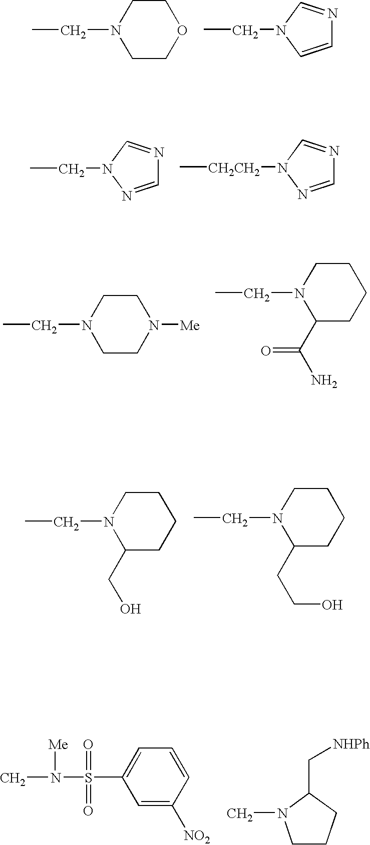

In one preferred embodiment, R² is H, halogen, OR¹³ or (CH₂)ₘR¹².

Even more preferably, R² is selected from H, Cl, OMe, OEt

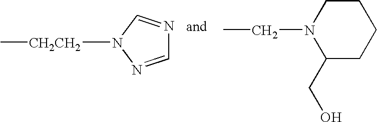

In one particularly preferred embodiment, R¹ is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph,

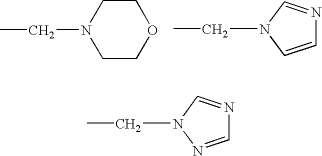

In one preferred embodiment, R⁷, R⁸, R⁹, and R¹⁰ are each independently selected from H, halogen, NO₂, CN, OH, NH₂, NHCOMe, CF₃, COMe, Me, Et, ⁱPr, NHMe, NMe₂, CONHMe, CONMe₂, SO₂NH₂, SO₂NHMe, SO₂NMe₂, SO₂Me, OMe, OEt, OCH₂CH₂OH, OCH₂CH₂OMe, CH₂OH, morpholino, piperidinyl, and piperazinyl.

In one preferred embodiment, R⁶ and R⁹ are both H.

In one preferred embodiment, R⁷ is selected from H, NO₂, NR¹³R¹⁴, OR¹³, CN, CF₃, CH₂OR¹³, SO₂R¹³ and halogen.

In a more preferred embodiment, R⁷ is selected from H, NO₂, NH₂, OH, OMe, CN, CH₂OH, F, CF₃ and SO₂Me.

In one preferred embodiment, R⁸ is selected from H, OR¹³, NO₂, OCH₂CH₂OMe, halogen, NR¹³R¹⁴, N-morpholino and OR¹³.

In a more preferred embodiment, R⁸ is selected from H, OH, NO₂, OCH₂CH₂OMe, Cl, F, NMe₂, N-morpholino, Me and OMe.

In another particularly preferred embodiment, R⁷, R⁸, R⁹, and R¹⁰ are each independently selected from H, halogen, NO₂, CN, OH, NH₂, NHCOMe, CF₃, COMe, Me, Et, ⁱPr, NHMe, NMe₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl, and piperazinyl.

Preferably, R⁷, R⁸ and R⁹ are each independently selected from H, halogen, NO₂, CN, OR¹³, NR¹³R¹⁴, NHCOR¹³, CF₃, COR¹³, R¹³, CONR¹³R¹⁴, SO₂NR¹³R¹⁴, SO₂R¹³, OR¹³, NR¹³SO₂R¹⁴, OCH₂CH₂OH, OCH₂CH₂OMe, morpholino, piperidinyl and piperazinyl.

Preferably,
R² is H or halogen;
R⁴ is H or OR³;
R⁶ and R⁹ are both H;
R⁷ is selected from H, NO₂, NR¹³R¹⁴, OR¹³ and CN;
R⁸ is selected from H, OR¹³, NO₂, OCH₂CH₂OMe, halogen, NR¹³R¹⁴, N-morpholino and OMe.

Preferably, where Z is CR¹⁰ and R⁹ is H, at least two of R⁷, R⁸ and R¹⁰ are other than OMe.

In yet another particularly preferred embodiment,
R² is H or Cl;
R⁴ is H or OMe;
R⁷ is selected from H, NO₂, NH₂, OH, OMe and CN; and
R⁸ is selected from H, OH, NO₂, OCH₂CH₂OMe, Cl, F, NMe₂, N-morpholino.

In one preferred embodiment, Z is CR¹⁰.

Preferably, R¹⁰ is selected from H, halogen, NO₂, CN, OR¹³, NR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, morpholino, piperidinyl and piperazinyl.

More preferably, R¹⁰ is selected from NO₂, NH₂, H, OH, OMe, CN, F, CH₂OH, CF₂ and SO₂Me.

More preferably still, R¹⁰ is H.

In another preferred embodiment, Z is N.

Another aspect of the invention relates to a compound selected from the following:
4-[4-(3-Nitro-phenyl)-pyrimidin-2-ylamino]-phenol [1];
(4-Nitro-phenyl)-[4-(3-nitro-phenyl)-pyrimidin-2-yl]-amine [2];
[4-(3-Amino-phenyl)-pyrimidin-2-yl]-[4-(2-methoxy-ethoxy)-phenyl]-amine [3];
[4-(3-Amino-phenyl)-pyrimidin-2-yl]-(4-nitro-phenyl)-amine [4];
(3-Nitro-phenyl)-[4-(3-nitro-phenyl)-pyrimidin-2-yl]-amine [5];
(4-Fluoro-phenyl)-[4-(3-nitro-phenyl)-pyrimidin-2-yl]-amine [6];
[4-(3-Amino-phenyl)-pyrimidin-2-yl]-(4-fluoro-phenyl)-amine [7];
N-[4-(3-Amino-phenyl)-pyrimidin-2-yl]-benzene-1,3-diamine [8];
N,N-Dimethyl-N′-[4-(3-nitro-phenyl)-pyrimidin-2-yl]-benzene-1,4-diamine [9];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
N-{3-[2-(4-Hydroxy-phenylamino)-pyrimidin-4-yl]-phenyl}-N-methyl-acetamide [12];
N-{3-[2-(4-Hydroxy-phenylamino)-pyrimidin-4-yl]-phenyl}-N-isobutyl-acetamide [13];
4-[4-(3-Methylamino-phenyl)-pyrimidin-2-ylamino]-phenol [14];
4-[4-(3-Amino-phenyl)-pyrimidin-2-ylamino]-phenol [15];
(4-Chloro-phenyl)-[4-(3-chloro-phenyl)-pyrimidin-2-yl]-amine [16];
4-[4-(3-Chloro-phenyl)-pyrimidin-2-ylamino]-phenol [17];
3-[4-(3-Chloro-phenyl)-pyrimidin-2-ylamino]-phenol [18]
[4-(3-Amino-phenyl)-pyrimidin-2-yl]-(3-nitro-phenyl)-amine [19];
N-[4-(3,4-Dichloro-phenyl)-pyrimidin-2-yl]-N′,N′-dimethyl-benzene-1,4-diamine [20];
4-[4-(3,4-Dichloro-phenyl)-pyrimidin-2-ylamino]-phenol [21];
3-[4-(3,4-Dichloro-phenyl)-pyrimidin-2-ylamino]-phenol [22];
N-Ethyl-N-{3-[2-(4-methoxy-phenylamino)-pyrimidin-4-yl]-phenyl}-acetamide [23];
N-Ethyl-N-{3-[2-(4-nitro-phenylamino)-pyrimidin-4-yl]-phenyl}-acteamide [24];
[4-(3-Ethylamino-phenyl)-pyrimidin-2-yl]-(4-methoxy-phenyl)-amine [25];
[4-(3-Ethylamino-phenyl)-pyrimidin-2-yl]-(4-nitro-phenyl)-amine [26];
{4-[3-(Benzylamino-methyl)-phenyl]-pyrimidin-2-yl}-(3-nitro-phenyl)-amine [27];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
[4-(3,4-Dichloro-phenyl)-pyrimidin-2-yl]-(3-nitro-phenyl)-amine [31];
(4-Morpholin-4-yl-phenyl)-[4-(3-[1,2,4]triazol-1-ylmethyl-phenyl)-pyrimidin-2-yl]-amine [32];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35];
3-[4-(3-[1,2,4]triazol-1-ylmethyl-phenyl)-pyrimidin-2-ylamino]-benzonitrile [36]
Phenyl-(4-phenyl-pyrimidin-2-yl)-amine [37];
[4-(5-Fluoro-2-methoxy-phenyl)-pyrimidin-2-yl]-phenyl-amine [38];
[4-(3-Morpholin-4-ylmethyl-phenyl)-pyrimidin-2-yl]-(3-nitro-phenyl)-amine [39];
N-{3-[2-(3-Nitro-phenylamino)-pyrimidin-4-yl]-benzyl}-methanesulfonamide [40];
(4-Nitro-phenyl)-[4-(3-[1,2,4]triazol-1-ylmethyl-phenyl)-pyrimidin-2-yl]-amine [41];
(4-Methoxy-phenyl)-[4-(3-[1,2,4]triazol-1-ylmethyl-phenyl)-pyrimidin-2-yl]-amine [42];
N,N-Dimethyl-N′-[4-(3-[1,2,4]triazol-1-ylmethyl-phenyl)-pyrimidin-2-yl]-benzene-1,4-diamine [43];
[4-(2,5-Dimethoxy-phenyl)-pyrimidin-2-yl]-(3-nitro-phenyl)-amine [44];
4-[4-(2,5-Dimethoxy-phenyl)-pyrimidin-2-ylamino]-phenol [45];
(4-{3-[(Ethyl-isopropyl-amino)-methyl]-phenyl}-pyrimidin-2-yl)-(3-nitro-phenyl)-amine [46];
[4-(4-Chloro-3-[1,2,4]triazol-1-ylmethyl-phenyl)-pyrimidin-2-yl]-(3-nitro-phenyl)-amine [47];
{4-[3-(Benzylamino-methyl)-phenyl]-pyrimidin-2-yl}-(6-chloro-pyridin-3-yl)-amine [48];
[4-(3,4-Dichloro-phenyl)-pyrimidin-2-yl]-(6-methoxy-pyridin-3-yl)-amine [49];
(6-Methoxy-pyridin-3-yl)-[4-(3-[1,2,4]triazol-1-ylmethyl-phenyl)-pyrimidin-2-yl]-amine [50];
3-[2-(6-Methoxy-pyridin-3-ylamino)-pyrimidin-4-yl]-benzonitrile [51];
[4-(2,5-Dimethoxy-phenyl)-pyrimidin-2-yl]-(6-methoxy-pyridin-3-yl)-amine [52];
(4-{3-[(Ethyl-isopropyl-amino)-methyl]-phenyl}-pyrimidin-2-yl)-(6-methoxy-pyridin-3-yl)-amine [53];
{4-[3-(4-Methyl-piperazin-1-ylmethyl)-phenyl]-pyrimidin-2-yl}-(3-nitro-phenyl)-amine [54];
3-[2-(3-Nitro-phenylamino)-pyrimidin-4-yl]-phenol [55];
[3-[2-(3-Hydroxy-phenylamino)-pyrimidin-4-yl]-phenol [56];
3-[2-(3-Fluoro-phenylamino)-pyrimidin-4-yl]-phenol [57];
(6-Methoxy-pyridin-3-yl)-{4-[3-(4-methyl-piperazin-1-ylmethyl)-phenyl]-pyrimidin-2-yl}-amine [58];
[4-(3-Imidazol-1-ylmethyl-phenyl)-pyrimidin-2-yl]-(6-methoxy-pyridin-3-yl)-amine [59];
N-{3-[2-(3-Hydroxymethyl-phenylamino)-pyrimidin-4-yl]-phenyl}-acetamide [60];
[4-(2,5-Dimethyl-phenyl)-pyrimidin-2-yl]-(3-nitro-phenyl)-amine [61];
3-[4-(2,5-Dimethyl-phenyl)-pyrimidin-2-ylamino]-phenol [62];
[4-(2,5-Dimethyl-phenyl)-pyrimidin-2-yl]-(3-fluoro-phenyl)-amine [63];
3-[4-(3-Nitro-phenyl)-pyrimidin-2-ylamino]-phenol [64];
(3-Fluoro-phenyl)-[4-(3-nitro-phenyl)-pyrimidin-2-yl]-amine [65];
N-[3-(2-Phenylamino-pyrimidin-4-yl)-phenyl]-acetamide [66];
N-{3-[2-(3-Hydroxy-phenylamino)-pyrimidin-4-yl]-phenyl}-acetamide [67];
N-{3-[2-(3,5-Dimethoxy-phenylamino)-pyrimidin-4-yl]-phenyl}-acetamide [68];
N-{3-[2-(3-Nitro-phenylamino)-pyrimidin-4-yl]-phenyl}-acetamide [69];
N-{3-[2-(Pyridin-3-ylamino)-pyrimidin-4-yl]-phenyl}-acetamide [70];
[4-(3-Dimethylaminomethyl-phenyl)-pyrimidin-2-yl]-(3-nitro-phenyl)-amine [71];
3-[2-(3-Hydroxymethyl-phenylamino)-pyrimidin-4-yl]-phenol [72];
3-[2-(Pyridin-3-ylamino)-pyrimidin-4-yl]-phenol [73];
3-[2-(6-Methoxy-pyridin-3-ylamino)-pyrimidin-4-yl]-phenol [74];
3-[2-(3,5-Bis-trifluoromethyl-phenylamino)-pyrimidin-4-yl]-phenol [75];
3-[4-(4-Methoxy-phenyl)-pyrimidin-2-ylamino]-phenol [76];
[4-(3-Methoxy-phenyl)-pyrimidin-2-yl]-(6-methoxy-pyridin-3-yl)-amine [77];
N-Isopropyl-N-{3-[2-(3-nitro-phenylamino)-pyrimidin-4-yl]-benzyl}-acetamide [78];
(1-{3-[2-(3-Nitro-phenylamino)-pyrimidin-4-yl]-benzyl}-piperidin-2-yl)-methanol [79];
3-[4-(3-Dimethylaminomethyl-phenyl)-pyrimidin-2-ylamino]-phenol [80];
4-[4-(3-Dimethylaminomethyl-phenyl)-pyrimidin-2-ylamino]-phenol [81];
[4-(3-Dimethylaminomethyl-phenyl)-pyrimidin-2-yl]-(4-morpholin-4-yl-phenyl)-amine [82];
[4-(3-Dimethylaminomethyl-phenyl)-pyrimidin-2-yl]-(6-methoxy-pyridin-3-yl)-amine [83];
[4-(3-Dimethylaminomethyl-phenyl)-pyrimidin-2-yl]-(3-nitro-phenyl)-amine [84];
N-Methyl-3-nitro-N-{3-[2-(3-nitro-phenylamino)-pyrimidin-4-yl]-benzyl}-benzenesulfonamide [85];
(3-Nitro-phenyl)-{4-[3-(2-phenylaminomethyl-pyrrolidin-1-ylmethyl)-phenyl]-pyrimidin-2-yl}-amine [86];
[4-(3-Methoxy-phenyl)-pyrimidin-2-yl]-(3-nitro-phenyl)-amine [87];
3-[4-(3-Methoxy-phenyl)-pyrimidin-2-ylamino]-phenol [88];
4-[4-(3,4-Dimethoxy-phenyl)-pyrimidin-2-ylamino]-phenol [89];
[4-(3,4-Dimethoxy-phenyl)-pyrimidin-2-yl]-(3-nitro-phenyl)-amine [90];
{3-[2-(3-Nitro-phenylamino)-pyrimidin-4-yl]-phenyl}-methanol [91];
3-[2-(3-Hydroxy-phenylamino)-pyrimidin-4-yl]-benzonitrile [92];
3-[2-(4-Hydroxy-phenylamino)-pyrimidin-4-yl]-benzonitrile [93];
[4-(4-Methoxy-phenyl)-pyrimidin-2-yl]-(3-nitro-phenyl)-amine [94];
3-[4-(3-trifluoromethyl-phenyl)-pyrimidin-2-ylamino]-phenol [95];
4-[4-(3-trifluoromethyl-phenyl)-pyrimidin-2-ylamino]-phenol [96];
(3-Nitro-phenyl)-[4-(3-trifluoromethyl-phenyl)-pyrimidin-2-yl]-amine [97];
4-[4-(3-Methoxy-phenyl)-pyrimidin-2-ylamino]-phenol [98];
1-{3-[2-(3-Nitro-phenylamino)-pyrimidin-4-yl]-benzyl}-piperidine-3-carboxylic acid amide [99];
2-(1-{3-[2-(3-Nitro-phenylamino)-pyrimidin-4-yl]-benzyl}-piperidin-3-yl)-ethanol [100];
(1-{3-[2-(4-Morpholin-4-yl-phenylamino)-pyrimidin-4-yl]-benzyl}-piperidin-2-yl)-methanol [101];
(1-{3-[2-(6-Methoxy-pyridin-3-ylamino)-pyrimidin-4-yl]-benzyl}-piperidin-2-yl)-methanol [102];
3-{4-[3-(2-Hydroxymethyl-piperidin-1-ylmethyl)-phenyl]-pyrimidin-2-ylamino}-phenol [103];
(3-Methanesulfonyl-phenyl)-[4-(3-[1,2,4]triazol-1-ylmethyl-phenyl)-pyrimidin-2-yl]-amine [104];
(1-{3-[2-(3-Nitro-phenylamino)-pyrimidin-4-yl]-benzyl}-piperidin-3-yl)-methanol [105];
4-{4-[3-(2-Hydroxymethyl-piperidin-1-ylmethyl)-phenyl]-pyrimidin-2-ylamino}-phenol [106];
(1-{3-[2-(3,5-Bis-hydroxymethyl-phenylamino)-pyrimidin-4-yl]-benzyl}-piperidin-2-yl)-methanol [107];
(1-{3-[2-(4-Methyl-3-nitro-phenylamino)-pyrimidin-4-yl]-benzyl}-piperidin-2-yl)-methanol [108];
3-[4-(4-Ethoxy-phenyl)-pyrimidin-2-ylamino]-phenol [109];
4-[4-(4-Methoxy-phenyl)-pyrimidin-2-ylamino]-phenol [110];
[4-(4-Methoxy-phenyl)-pyrimidin-2-yl]-(4-morpholin-4-yl-phenyl)-amine [111];
[4-(3-Chloro-phenyl)-pyrimidin-2-yl]-(3-nitro-phenyl)-amine [112];
4-[4-(3-Fluoro-phenyl)-pyrimidin-2-ylamino]-phenol [113];
3-[4-(2,5-Difluoro-phenyl)-pyrimidin-2-ylamino]-phenol [114];
3-[4-(3-Hydroxymethyl-phenyl)-pyrimidin-2-ylamino]-phenol [115];
{3-[2-(3-Fluoro-phenylamino)-pyrimidin-4-yl]-phenyl}-methanol [116];
{3-[2-(3,5-Dinitro-phenylamino)-pyrimidin-4-yl]-phenyl}-methanol [117];
(3-Fluoro-phenyl)-[4-(3-methoxy-phenyl)-pyrimidin-2-yl]-amine [118];
(3-Fluoro-phenyl)-[4-(4-methoxy-phenyl)-pyrimidin-2-yl]-amine [119];
3-[2-(3,4,5-trimethoxy-phenylamino)-pyrimidin-4-yl]-phenol [120];
3-[2-(3,5-Dimethoxy-phenylamino)-pyrimidin-4-yl]-phenol [121];
3-[2-(4-Hydroxy-phenylamino)-pyrimidin-4-yl]-phenol [122];
[4-(2,5-Difluoro-phenyl)-pyrimidin-2-yl]-(3-nitro-phenyl)-amine [123];
[4-(4-Methoxy-phenyl)-pyrimidin-2-yl]-(6-methoxy-pyridin-3-yl)-amine [124];
{3-[2-(6-Methoxy-pyridin-3-ylamino)-pyrimidin-4-yl]-phenyl}-methanol [125];
(3-Nitro-phenyl)-{4-[4-(2-[1,2,4]triazol-1-yl-ethyl)-phenyl]-pyrimidin-2-yl}-amine [126];
(1-{4-[2-(3-Nitro-phenylamino)-pyrimidin-4-yl]-benzyl}-piperidin-2-yl)-methanol [127];
[4-(4-Methoxy-phenyl)-pyrimidin-2-yl]-(3,4,5-trimethoxy-phenyl)-amine [128];
N-Methyl-N-{3-[2-(3-nitro-phenylamino)-pyrimidin-4-yl]-phenyl}-methanesulfonamide [129];
N-{3-[2-(3-Hydroxy-phenylamino)-pyrimidin-4-yl]-phenyl}-N-methyl-methanesulfonamide [130];
N-{3-[2-(4-Hydroxy-phenylamino)-pyrimidin-4-yl]-phenyl}-N-methyl-methanesulfonamide [131]; and
N-{3-[2-(6-Methoxy-pyridin-3-ylamino)-pyrimidin-4-yl]-phenyl}-N-methyl-methanesulfonamide [132].

In one embodiment, the present invention relates to a compound selected from the following:
4-[4-(3-Nitro-phenyl)-pyrimidin-2-ylamino]-phenol [1];
(4-Nitro-phenyl)-[4-(3-nitro-phenyl)-pyrimidin-2-yl]-amine [2];
[4-(3-Amino-phenyl)-pyrimidin-2-yl]-[4-(2-methoxy-ethoxy)-phenyl]-amine [3];
[4-(3-Amino-phenyl)-pyrimidin-2-yl]-(4-nitro-phenyl)-amine [4];
(3-Nitro-phenyl)-[4-(3-nitro-phenyl)-pyrimidin-2-yl]-amine [5];
(4-Fluoro-phenyl)-[4-(3-nitro-phenyl)-pyrimidin-2-yl]-amine [6];
[4-(3-Amino-phenyl)-pyrimidin-2-yl]-(4-fluoro-phenyl)-amine [7];
N-[4-(3-Amino-phenyl)-pyrimidin-2-yl]-benzene-1,3-diamine [8];
N,N-Dimethyl-N′-[4-(3-nitro-phenyl)-pyrimidin-2-yl]-benzene-1,4-diamine [9];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl-acetamide [11];
N-{3-[2-(4-Hydroxy-phenylamino)-pyrimidin-4-yl]-phenyl}-N-methyl-acetamide [12];
N-{3-[2-(4-Hydroxy-phenylamino)-pyrimidin-4-yl]-phenyl}-N-isobutyl-acetamide [13];
4-[4-(3-Methylamino-phenyl)-pyrimidin-2-ylamino]-phenol [14];
4-[4-(3-Amino-phenyl)-pyrimidin-2-ylamino]-phenol [15];
(4-Chloro-phenyl)-[4-(3-chloro-phenyl)-pyrimidin-2-yl]-amine [16];
4-[4-(3-Chloro-phenyl)-pyrimidin-2-ylamino]-phenol [17];
3-[4-(3-Chloro-phenyl)-pyrimidin-2-ylamino]-phenol [18];
[4-(3-Amino-phenyl)-pyrimidin-2-yl]-(3-nitro-phenyl)-amine [19];
N-[4-(3,4-Dichloro-phenyl)-pyrimidin-2-yl]-N′,N′-dimethyl-benzene-1,4-diamine [20];
4-[4-(3,4-Dichloro-phenyl)-pyrimidin-2-ylamino]-phenol [21];
3-[4-(3,4-Dichloro-phenyl)-pyrimidin-2-ylamino]-phenol [22];
N-Ethyl-N-{3-[2-(4-methoxy-phenylamino)-pyrimidin-4-yl]-phenyl}-acetamide [23];
N-Ethyl-N-{3-[2-(4-nitro-phenylamino)-pyrimidin-4-yl]-phenyl}-acetamide [24];
[4-(3-Ethylamino-phenyl)-pyrimidin-2-yl]-(4-methoxy-phenyl)-amine [25];
[4-(3-Ethylamino-phenyl)-pyrimidin-2-yl]-(4-nitro-phenyl)-amine [26];
{4-[3-(Benzylamino-methyl)-phenyl]-pyrimidin-2-yl}-(3-nitro-phenyl)-amine [27];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
[4-(3,4-Dichloro-phenyl)-pyrimidin-2-yl]-(3-nitro-phenyl)-amine [31];
(4-Morpholin-4-yl-phenyl)-[4-(3-[1,2,4]triazol-1-ylmethyl-phenyl)-pyrimidin-2-yl]-amine [32];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1 ,2,4]triazol-1-ylmethyl-phenyl)-pyrimidin-2-yl]-amine [35];
3-[4-(3-[1,2,4]triazol-1-ylmethyl-phenyl)-pyrimidin-2-ylamino]-benzonitrile [36];
Phenyl-(4-phenyl-pyrimidin-2-yl)-amine [37];
[4-(5-Fluoro-2-methoxy-phenyl)-pyrimidin-2-yl]-phenyl-amine [38];
[4-(3-Morpholin-4-ylmethyl-phenyl)-pyrimidin-2-yl]-(3-nitro-phenyl)-amine [39];
N-{3-[2-(3-Nitro-phenylamino)-pyrimidin-4-yl]-benzyl}-methanesulfonamide [40];
(4-Nitro-phenyl)-[4-(3-[1,2,4]triazol-1-ylmethyl-phenyl)-pyrimidin-2-yl]-amine [41];
(4-Methoxy-phenyl)-[4-(3-[1,2,4]triazol-1-ylmethyl-phenyl)-pyrimidin-2-yl]-amine [42];
N,N-Dimethyl-N′-[4-(3-[1,2,4]triazol-1-ylmethyl-phenyl)-pyrimidin-2-yl]-benzene-1,4-diamine [43];
[4-(2,5-Dimethoxy-phenyl)-pyrimidin-2-yl]-(3-nitro-phenyl)-amine [44];
4-[4-(2,5-Dimethoxy-phenyl)-pyrimidin-2-ylamino]-phenol [45];
(4-{3-[(Ethyl-isopropyl-amino)-methyl]-phenyl}-pyrimidin-2-yl)-(3-nitro-phenyl)-amine [46];
[4-(4-Chloro-3-[1,2,4]triazol-1-ylmethyl-phenyl)-pyrimidin-2-yl]-(3-nitro-phenyl)-amine [47];
{4-[3-(Benzylamino-methyl)-phenyl]-pyrimidin-2-yl}-(6-chloro-pyridin-3-yl)-amine [48];
[4-(3,4-Dichloro-phenyl)-pyrimidin-2-yl]-(6-methoxy-pyridin-3-yl)-amine [49];
(6-Methoxy-pyridin-3-yl)-[4-(3-[1,2,4]triazol-1-ylmethyl-phenyl)-pyrimidin-2-yl]-amine [50];
3-[2-(6-Methoxy-pyridin-3-ylamino)-pyrimidin-4-yl]-benzonitrile [51];
[4-(2,5-Dimethoxy-phenyl)-pyrimidin-2-yl]-(6-methoxy-pyridin-3-yl)-amine [52]; and
(4-3-[(Ethyl-isopropyl-amino)-methyl]-phenyl}-pyrimidin-2-yl)-(6-methoxy-pyridin-3-yl)-amine [53].

In another particularly preferred embodiment of the invention, the compound is selected from [3], [10], [11], [26], [29], [30], [34], [39], [40], [44], [46], [53], [54], [58], [78], [79], [80], [81], [82], [83], [99], [100] and [103].

In another particularly preferred embodiment of the invention, the compound is selected from [3], [26], [29], [40], [44], [46], [53], [54], [78], [79], [80], [81], [83], [99] and [100].

In another particularly preferred embodiment of the invention, the compound is selected from [26], [44], [46], [54], [79], [83] and [100].

In another particularly preferred embodiment of the invention, the compound is selected from [46], [79] and [100].

In one preferred embodiment, the compound of the invention is capable of inhibiting one or more protein kinases selected from CDK1/cyclin B, CDK2/cyclin A, CDK2/cyclin E, CDK4/cyclin D1, CDK7/cyclin H, CDK9/cyclin T1, GSK3β, aurora kinase and PLK1, as measured by the appropriate assay. In one particularly preferred embodiment, the compound of the invention exhibits an IC₅₀ value for kinase inhibition of less than 10 μM, more preferably less than 1 μM, more preferably still less than 0.1 μM. Compounds falling within each of these preferred embodiments can be identified from Table 1, which shows the IC₅₀ values for compounds [1]-[134]. Details of the various kinase assays are disclosed in the accompanying Examples section.

In one preferred embodiment, the invention relates to compounds that are capable of exhibiting an antiproliferative effect against one or more transformed human cell lines in vitro as measured by a 72-h MTT cytotoxicity assay. In a particularly preferred embodiment, the compound of the invention exhibits an IC₅₀ value (average) of less than 10 μM against one or more transformed human cell lines in vitro as measured by a 72-h MTT cytotoxicity assay. More preferably, the compound exhibits an IC₅₀ value (average) of less than 5 μM, more preferably still, less than 1 μM. Compounds falling within each of these preferred embodiments can be identified from Table 2, which shows the IC₅₀ values for selected compounds of the invention. Details of the various cytotoxicity assays are disclosed in the accompanying Examples section.

In a preferred embodiment, the invention relates to compounds that are capable of exhibiting an antiproliferative effect against one or more transformed human cell lines in vitro, wherein said compound is selected from the following:
[4-(3-Amino-phenyl)-pyrimidin-2-yl]-[4-(2-methoxy-ethoxy)-phenyl]-amine [3];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
(4-Morpholin-4-yl-phenyl)-[4-(3-[1,2,4]triazol-1-ylmethyl-phenyl)-pyrimidin-2-yl]-amine [32];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35]; and
(6-Methoxy-pyridin-3-yl)-[4-(3-[1,2,4]triazol-1-ylmethyl-phenyl)-pyrimidin-2-yl]-amine [50].

Even more preferably, the compound of the invention is capable of exhibiting an IC₅₀ value (average) of less than 10 μM against one or more transformed human cell lines in vitro as measured by a 72-h MTT cytotoxicity assay. Preferably, the compound is selected from the following:
[4-(3-Amino-phenyl)-pyrimidin-2-yl]-[4-(2-methoxy-ethoxy)-phenyl]-amine [3];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30]; and
3-[4-(3-[1,2,4]triazol-1-ylmethyl-phenyl)-pyrimidin-2-ylamino]-phenol [34].

Even more preferably still, the compound of the invention is capable of exhibiting an IC₅₀ value (average) of less than 5 μM against one or more transformed human cell lines in vitro as measured by a 72-h MTT cytotoxicity assay. Preferably, the compound is selected from:
[4-(3-Amino-phenyl)-pyrimidin-2-yl]-[4-(2-methoxy-ethoxy)-phenyl]-amine [3]; and
[4-(3-Imidazol-1-ylmethyl-phenyl)-pyrimidin-2-yl]-(3-nitro-phenyl)-amine [29].

In another preferred embodiment, the compound of the invention is capable of inhibiting one or more protein kinases selected from CDK1/cyclin B, CDK2/cyclin A, CDK2/cyclin E, CDK4/cyclin D1, CDK7/cyclin H, CDK9/cyclin T1, GSK3β, aurora kinase and PLK1, as measured by the appropriate assay. Preferably, the compound is selected from the following:
4-[4-(3-Nitro-phenyl)-pyrimidin-2-ylamino]-phenol [1];
[4-(3-Amino-phenyl)-pyrimidin-2-yl]-[4-(2-methoxy-ethoxy)-phenyl]-amine [3];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
N-{3-[2-(4-Hydroxy-phenylamino)-pyrimidin-4-yl]-phenyl}-N-methyl-acetamide [12];
N-{3-[2-(4-Hydroxy-phenylamino)-pyrimidin-4-yl]-phenyl}-N-isobutyl-acetamide [13];
4-[4-(3-Methylamino-phenyl)-pyrimidin-2-ylamino]-phenol [14];
4-[4-(3-Amino-phenyl)-pyrimidin-2-ylamino]-phenol [15];
4-[4-(3-Chloro-phenyl)-pyrimidin-2-ylamino]-phenol [17];
3-[4-(3-Chloro-phenyl)-pyrimidin-2-ylamino]-phenol [18];
4-[4-(3,4-Dichloro-phenyl)-pyrimidin-2-ylamino]-phenol [21];
{4-[3-(Benzylamino-methyl)-phenyl]-pyrimidin-2-yl}-(3-nitro-phenyl)-amine [27];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
(4-Morpholin-4-yl-phenyl)-[4-(3-[1,2,4]triazol-1-ylmethyl-phenyl)-pyrimidin-2-yl]-amine [32];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35];
3-[4-(3-[1,2,4]triazol-1-ylmethyl-phenyl)-pyrimidin-2-ylamino]-benzonitrile [36];
{4-[3-(Benzylamino-methyl)-phenyl]-pyrimidin-2-yl}-(6-chloro-pyridin-3-yl)-amine [48]; and
(6-Methoxy-pyridin-3-yl)-[4-(3-[1,2,4]triazol-1-ylmethyl-phenyl)-pyrimidin-2-yl]-amine [50].

More preferably, the compound exhibits an IC₅₀ value (for kinase inhibition) of less than 10 μM. Preferably, the compound is selected from the following:
[4-(3-Amino-phenyl)-pyrimidin-2-yl]-[4-(2-methoxy-ethoxy)-phenyl]-amine [3];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
N-{3-[2-(4-Hydroxy-phenylamino)-pyrimidin-4-yl]-phenyl}-N-methyl-acetamide [12];
4-[4-(3-Methylamino-phenyl)-pyrimidin-2-ylamino]-phenol [14];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35];
3-[4-(3-[1,2,4]triazol-1-ylmethyl-phenyl)-pyrimidin-2-ylamino]-benzonitrile [36]; and
(6-Methoxy-pyridin-3-yl)-[4-(3-[1,2,4]triazol-1-ylmethyl-phenyl)-pyrimidin-2-yl]-amine [50].

More preferably still, the compound of said second aspect exhibits an IC₅₀ value (for kinase inhibition) of less than 0.1 μM. Preferably, the compound is selected from the following:
[4-(3-Amino-phenyl)-pyrimidin-2-yl]-[4-(2-methoxy-ethoxy)-phenyl]-amine [3];
4-[4-(3-Methylamino-phenyl)-pyrimidin-2-ylamino]-phenol [14];
(3-Nitro-phenyl)-[4-(3-[1,2,4]triazol-1-ylmethyl-phenyl)-pyrimidin-2-yl]-amine [30]; and
3-[4-(3-[1,2,4]triazol-1-ylmethyl-phenyl)-pyrimidin-2-ylamino]-benzonitrile [36].
   Therapeutic Use

The compounds of the present invention have been found to possess anti-proliferative activity and are therefore believed to be of use in the treatment of proliferative disorders such as cancers, leukaemias and other disorders associated with uncontrolled cellular proliferation such as psoriasis and restenosis. As defined herein, an anti-proliferative effect within the scope of the present invention may be demonstrated by the ability to inhibit cell proliferation in an in vitro whole cell assay, for example using any of the cell lines A549, HT29 or Saos-2 Using such assays it may be determined whether a compound is anti-proliferative in the context of the present invention.

One preferred embodiment of the present invention therefore relates to the use of one or more compounds of the invention in the preparation of a medicament for treating a proliferative disorder.

As used herein the phrase “preparation of a medicament” includes the use of a compound of the invention directly as the medicament in addition to its use in a screening programme for further therapeutic agents or in any stage of the manufacture of such a medicament.

Preferably, the proliferative disorder is a cancer or leukaemia. The term proliferative disorder is used herein in a broad sense to include any disorder that requires control of the cell cycle, for example cardiovascular disorders such as restenosis, cardiomyopathy and myocardial infarction, auto-immune disorders such as glomerulonephritis and rheumatoid arthritis, dermatological disorders such as psoriasis, anti-inflammatory, anti-fungal, antiparasitic disorders such as malaria, emphysema, alopecia, and chronic obstructive pulmonary disorder. In these disorders, the compounds of the present invention may induce apoptosis or maintain stasis within the desired cells as required.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment of the invention, the compound of the invention is administered in an amount sufficient to inhibit at least one CDK enzyme.

Preferably, the compound of the invention is administered in an amount sufficient to inhibit at least one of CDK2 and/or CDK4.

Another aspect of the invention relates to the use of a compound of the invention in the preparation of a medicament for treating a viral disorder, such as human cytomegalovirus (HCMV), herpes simplex virus type 1 (HSV-1), human immunodeficiency virus type 1 (HIV-1), and varicella zoster virus (VZV).

In a more preferred embodiment of the invention, the compound of the invention is administered in an amount sufficient to inhibit one or more of the host cell CDKs involved in viral replication, i.e. CDK2, CDK7, CDK8, and CDK9 [23].

As defined herein, an anti-viral effect within the scope of the present invention may be demonstrated by the ability to inhibit CDK2, CDK7, CDK8 or CDK9.

In a particularly preferred embodiment, the invention relates to the use of one or more compounds of the invention in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

A further aspect of the invention relates to a method of treating a CDK-dependent disorder, said method comprising administering to a subject in need thereof, a compound according to the invention, or a pharmaceutically acceptable salt thereof, as defined above in an amount sufficient to inhibit a cyclin dependent kinase.

Preferably, the CDK-dependent disorder is a viral disorder or a proliferative disorder, more preferably cancer.

Another aspect of the invention relates to the use of compounds of the invention, or pharmaceutically accetable salts thereof, in the preparation of a medicament for treating diabetes.

In a particularly preferred embodiment, the diabetes is type II diabetes.

GSK3 is one of several protein kinases that phosphorylate glycogen synthase (GS). The stimulation of glycogen synthesis by insulin in skeletal muscle results from the dephosphorylation and activation of GS. GSK3's action on GS thus results in the latter's deactivation and thus suppression of the conversion of glucose into glycogen in muscles.

Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes [24]. Furthermore, it has been demonstrated that GSK3 is over-expressed in muscle cells of type II diabetics and that an inverse correlation exists between skeletal muscle GSK3 activity and insulin action [25].

GSK3 inhibition is therefore of therapeutic significance in the treatment of diabetes, particularly type II, and diabetic neuropathy.

It is notable that GSK3 is known to phosphorylate many substrates other than GS, and is thus involved in the regulation of multiple biochemical pathways. For example, GSK is highly expressed in the central and peripheral nervous systems.

Preferably, the compound is administered in an amount sufficient to inhibit GSK, more preferably GSK3, more preferably still GSK3β.

Another aspect of the invention therefore relates to the use of compounds of the invention, or pharmaceutically acceptable salts thereof, in the preparation of a medicament for treating a CNS disorders, for example neurodegenerative disorders.

Preferably, the CNS disorder is Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages.

Without wishing to be bound by theory, it is believed that GSK3 inhibitors may be able to prevent and/or reverse the abnormal hyperphosphorylation, of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process [26].

Another aspect of the invention relates to the use of compounds of the invention, or pharmaceutically acceptable salts thereof, in the preparation of a medicament for treating bipolar disorder.

Yet another aspect of the invention relates to the use of compounds of the invention, or pharmaceutically acceptable salts thereof, in the preparation of a medicament for treating a stroke.

Reducing neuronal apoptosis is an important therapeutic goal in the context of head trauma, stroke, epilepsy, and motor neuron disease [27]. Therefore, GSK3 as a pro-apoptotic factor in neuronal cells makes this protein kinase an attractive therapeutic target for the design of inhibitory drugs to treat these diseases.

Yet another aspect of the invention relates to the use of compounds of the invention, or pharmaceutically acceptable salts thereof, in the preparation of a medicament for treating alopecia.

Hair growth is controlled by the Wnt signalling pathway, in particular Wnt-3. In tissue-culture model systems of the skin, the expression of non-degradable mutants of β-catenin leads to a dramatic increase in the population of putative stem cells, which have greater proliferative potential [28]. This population of stem cells expresses a higher level of non-cadherin-associated β-catenin [29], which may contribute to their high proliferative potential. Moreover, transgenic mice overexpressing a truncated β-catenin in the skin undergo de novo hair-follicle morphogenesis, which normally is only established during embryogenesis. The ectopic application of GSK3 inhibitors may therefore be therapeutically useful in the treatment of baldness and in restoring hair growth following chemotherapy-induced alopecia.

A further aspect of the invention relates to a method of treating a GSK3-dependent disorder, said method comprising administering to a subject in need thereof, a compound according to the invention, or a pharmaceutically acceptable salt thereof, as defined above in an amount sufficient to inhibit GSK3.

Preferably, the GSK3-dependent disorder is diabetes.

Preferably, the compound of the invention, or pharmaceutically acceptable salt thereof, is administered in an amount sufficient to inhibit GSK30.

In one embodiment of the invention, the compound of the invention is administered in an amount sufficient to inhibit at least one PLK enzyme.

The polo-like kinases (PLKs) constitute a family of serine/threonine protein kinases. Mitotic Drosophila melanogaster mutants at the polo locus display spindle abnormalities [30] and polo was found to encode a mitotic kinase [31]. In humans, there exist three closely related PLKs [32]. They contain a highly homologous amino-terminal catalytic kinase domain and their carboxyl termini contain two or three conserved regions, the polo boxes. The function of the polo boxes remains incompletely understood but they are implicated in the targeting of PLKs to subcellular compartments [33,34], mediation of interactions with other proteins [35], or may constitute part of an autoregulatory domain [36]. Furthermore, the polo box-dependent PLK1 activity is required for proper metaphase/anaphase transition and cytokinesis [37,38].

Studies have shown that human PLKs regulate some fundamental aspects of mitosis [39,40]. In particular, PLK1 activity is believed to be necessary for the functional maturation of centrosomes in late G2/early prophase and subsequent establishment of a bipolar spindle. Depletion of cellular PLK1 through the small interfering RNA (siRNA) technique has also confirmed that this protein is required for multiple mitotic processes and completion of cytokinesis [41].

In a more preferred embodiment of the invention, the compound of the invention is administered in an amount sufficient to inhibit PLK1.

Of the three human PLKs, PLK1 is the best characterized; it regulates a number of cell division cycle effects, including the onset of mitosis [42,43], DNA-damage checkpoint activation [44,45], regulation of the anaphase promoting complex [46-48], phosphorylation of the proteasome [49], and centrosome duplication and maturation [50].

Specifically, initiation of mitosis requires activation of M-phase promoting factor (MPF), the complex between the cyclin dependent kinase CDK1and B-type cyclins [51]. The latter accumulate during the S and G2 phases of the cell cycle and promote the inhibitory phosphorylation of the MPF complex by WEE1, MIK1, and MYT1 kinases. At the end of the G2 phase, corresponding dephosphorylation by the dual-specificity phosphatase CDC25C triggers the activation of MPF [52]. In interphase, cyclin B localizes to the cytoplasm [53], it then becomes phosphorylated during prophase and this event causes nuclear translocation [54,55]. The nuclear accumulation of active MPF during prophase is thought to be important for initiating M-phase events [56]. However, nuclear MPF is kept inactive by WEE1 unless counteracted by CDC25C. The phosphatase CDC25C itself, localized to the cytoplasm during interphase, accumulates in the nucleus in prophase [57-59]. The nuclear entry of both cyclin B [60] and CDC25C [61] are promoted through phosphorylation by PLK1 [43]. This kinase is an important regulator of M-phase initiation.

In one particularly preferred embodiment, the compounds of the invention are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of the invention is administered in an amount sufficient to inhibit PLK2 and/or PLK3.

Mammalian PLK2 (also known as SNK) and PLK3 (also known as PRK and FNK) were originally shown to be immediate early gene products. PLK3 kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [62]. PLK2 is the least well understood homologue of the three PLKs. Both PLK2 and PLK3 may have additional important post-mitotic functions [35].

A further aspect of the invention relates to a method of treating a PLK-dependent disorder, said method comprising administering to a subject in need thereof, a compound according to the invention, or a pharmaceutically acceptable salt thereof, as defined above in an amount sufficient to inhibit PLK.

Preferably, the PLK-dependent disorder is a proliferative disorder, more preferably cancer.

Preferably, the compound of the invention, or pharmaceutically acceptable salt thereof, is administered in an amount sufficient to inhibit aurora kinase.

A further aspect of the invention relates to a method of treating an aurora kinase-dependent disorder, said method comprising administering to a subject in need thereof, a compound according to the invention, or a pharmaceutically acceptable salt thereof, as defined above in an amount sufficient to inhibit aurora kinase.

Preferably, the aurora kinase dependent disorder is a viral disorder as defined above.

Pharmaceutical Compositions

Another aspect of the invention relates to a pharmaceutical composition comprising a compound of the invention as defined above admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agent or pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the compounds of the present invention. The terms used in the claims encompass these forms.

Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient.

Combinations

In a particularly preferred embodiment, the one or more compounds of the invention are administered in combination with one or more other therapeutically active agents, for example, existing drugs available on the market. In such cases, the compounds of the invention may be administered consecutively, simultaneously or sequentially with the one or more other active agents.

By way of example, it is known that anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

Assays

Another aspect of the invention relates to the use of a compound of the invention in an assay for identifying further candidate compounds capable of inhibiting one or more protein kinases.

Another aspect of the invention relates to the use of a compound of the invention in an assay for identifying further candidate compounds capable of inhibiting one or more cyclin dependent kinases, aurora kinase, GSK and PLK.

Preferably, the assay is a competitive binding assay.

More preferably, the competitive binding assay comprises contacting a compound of the invention with a protein kinase and a candidate compound and detecting any change in the interaction between the compound of the invention and the protein kinase.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:
(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders, viral disorders, a CNS disorder, stroke, alopecia and diabetes.

Preferably, said candidate compound is generated by conventional SAR modification of a compound of the invention.

As used herein, the term “conventional SAR modification” refers to standard methods known in the art for varying a given compound by way of chemical derivatisation.

The above methods may be used to screen for a ligand useful as an inhibitor of one or more protein kinases.

Synthesis

The compounds of the invention can be prepared by any method known in the art. Two convenient synthetic routes are shown below in Scheme 1: 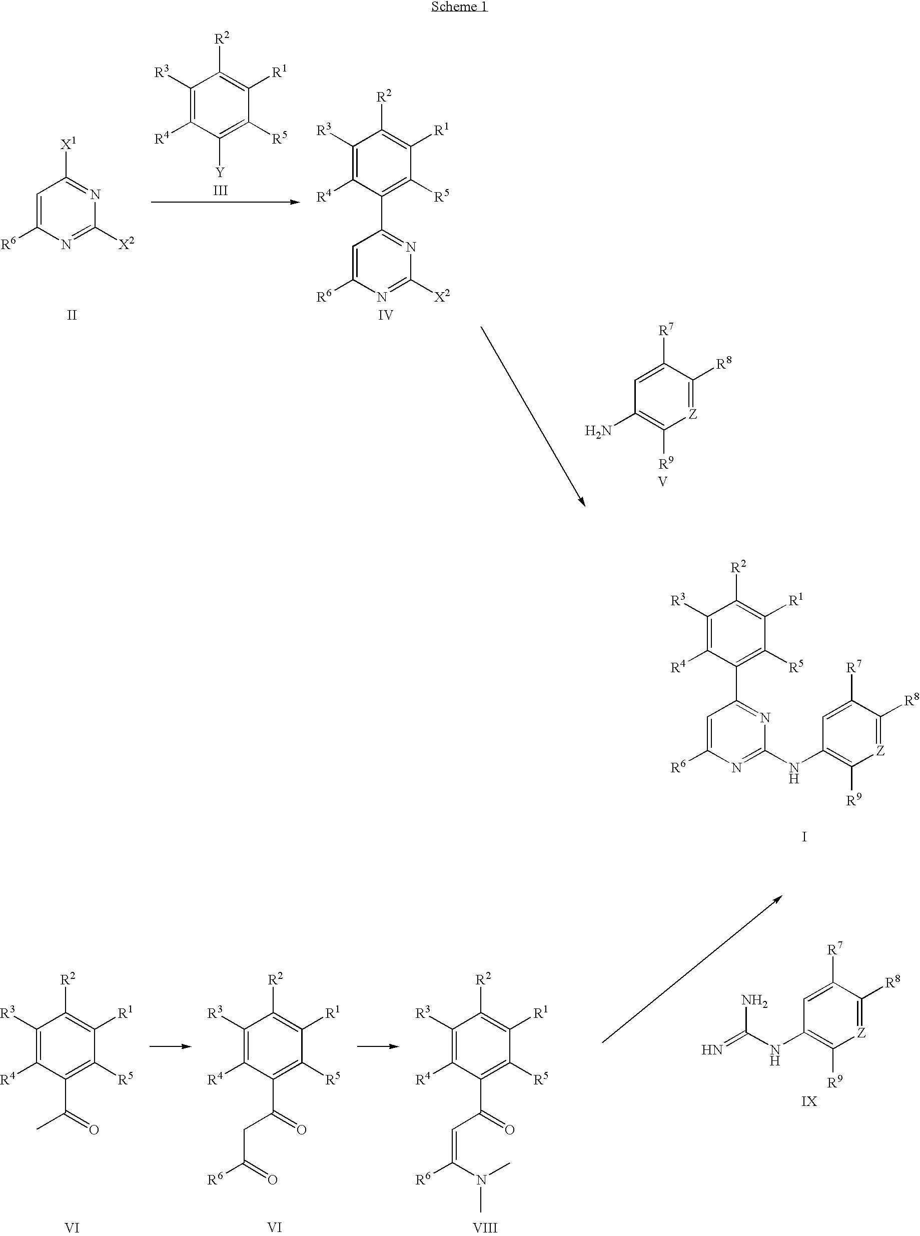
Palladium-catalysed cross-coupling of phenyl boronic acids (III, Y═B(OH)₂) or their derivatives with 2,4-dihalogenated pyrimidines (II; e.g. X¹═X²═Cl) [63, 64] affords 4-arylated 2-halogenopyrimidines IV, which are aminated with anilines V. Alternatively, acetophenones VI are acylated, e.g. with R⁶COCl, to provide the diketones VII. These in turn are enaminated to VIII [65], followed by condensation with arylguanidines IX [66].

A further aspect of the invention therefore relates to a process for preparing a compound of formula I as defined above, said process comprising the steps of: 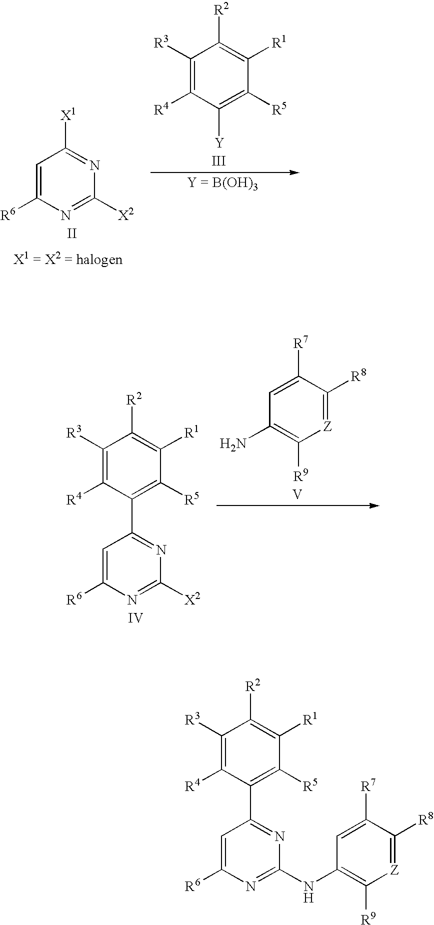
(i) reacting a phenyl boronic acid of formula III with a 2,4-dihalogenated pyrimidine of formula II to form a compound of formula IV; and
(ii) reacting said compound of formula IV with an aniline of formula V to form a compound of formula I.

Yet another aspect of the invention relates to a process for preparing a compound of formula I as defined above, said process comprising the steps of: 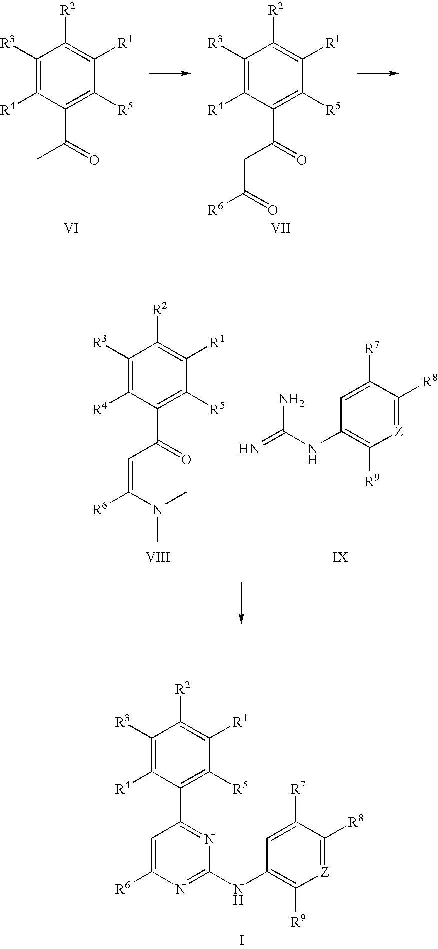
(i) reacting a compound of formula VI with R⁶COCl, where R⁶ is as defined above, to form a compound of formula VII;
(ii) converting said compound of formula VII to a compound of formula VIII; and
(iii) reacting said compound of formula VIII with a compound of formula IX to form a compound of formula I.

### EXAMPLES

### Example 1

General

HPLC retention times (t_{R}) were measured using Vydac 218TP54 columns (C₁₈ reversed-phase stationary phase; 4.5×250 mm columns), eluted at 1 mL/min with a linear gradient of acetonitrile in water (containing 0.1% CF₃COOH) as indicated, followed by isocratic elution. UV monitors (254 nm) were used. All purification work, unless otherwise stated, was performed using silica gel 60A (particle size 35-70 micron. ¹H-NMR spectra were recorded using 500 MHz instrument. Chemical shifts are given in ppm using TMS as standard and coupling constants (J) are stated in Hz. Mass spectra were recorded under positive or negative ion electrospray conditions.

The structures of selected compounds of the invention are shown in Table 1.

### Example 2

### [4-(3-Amino-phenyl)-pyrimidin-2-yl]-[4-(2-methoxy-ethoxy)-phenyl]-amine (3)

A mixture of 3-aminoacetophenone (1.35 g, 10 mmol) and N,N-dimethylformamide dimethylacetal (3.99 mL, 30 mmol) was heated at 102° C. for 8 h. On cooling, the reaction mixture was evaporated to dryness. The yellow residue was collected and washed with EtOAc/PE (1:5) to yield 1-(3-amino-phenyl)-3-dimethylamino-propenone as an orange solid (1.85 g, 97%). ¹H-NMR (CDCl₃): δ 2.41 (s, 6H, CH₃), 5.75 (d, 1H, J=12.0 Hz, CH), 6.88 (d, 1H, J=8.0 Hz, Ph-H), 6.98 (d, 1H, J=8.0 Hz, Ph-H), 7.14 (t, 1H, J=8.0 Hz, Ph-H), 7.38 (s, 1H, Ph-H), 7.57 (d, 1H, J=12.0 Hz, CH); MS (ESI⁺) m/z 191.22 [M+H]⁺, C₁₁H₁₄N₂O requires 190.24.

An aliquot of this material (0.73 g, 38.1 mmol), dissolved in 2-methoxylethanol (3 mL), was treated with N-(4-hydroxy-phenyl)-guanidine nitrate (0.82 g, 38.1 mmol), which was prepared by condensation of 4-amino-phenol and aqueous cyanamide solution in the presence of nitric acid, and NaOH (0.15 g, 38.1 mmol). After refluxing overnight, the reaction mixture was concentrated and the residue was purified by SiO₂ gel chromatography (EtOAc/PE, 5:1) to afford the title compound (85 mg, 7%). ¹H-NMR (CD₃OD): δ 3.36 (s, 3H OCH₃), 3.58 (t, 2H, J=5.0 Hz, CH₂), 4.13 (t, 2H, J=5.0 Hz, CH₂), 6.80 (d, 2H, J=8.0 Hz, Ph-H), 6.86 (d, 2H, J=8.0 Hz, Ph-H), 7.12 (d, 1H, J=5.0 Hz, pyrimidine-H), 7.22 (t, 1H, J=8.0 Hz, Ph-H), 7.41 (d, 1H, J=9.0 Hz, Ph-H), 7.45 (s, 1H, Ph-H), 7.47 (d, 1H, J=8.0 Hz, Ph-H), 8.32 (d, 1H, J=5.0 Hz, pyrimidine-H); MS (ESI⁺) m/z 336.80 [M]; C₁₉H₂₀N₄O₂ requires 336.39.

### Example 3

### N-Ethyl-N-{3-[2-(4-hydroxy-phenylamino)-pyrimidin-4-yl]-phenyl}-acetamide (10)

Acetamidoacetophenone (0.2 g, 1.13 mmol) in Me₂CO (2 mL) was treated with KOH (63 mg, 1.13 mmol) and then iodoethane (0.45 mL, 5.64 mmol). After stirring at room temperature overnight the reaction mixture was concentrated to dryness. The residue was redissolved in EtOAc and was washed with H₂O and brine, and was dried on MgSO₄. The solvent was evaporated to yield N-(3-acetyl-phenyl)-N-ethyl-acetamide as an orange powder (0.23 g, 100%): mp 203-204° C.; ¹H-NMR (CD₃OD): δ 1.11 (t, 3H, J=7.0 Hz, CH₃), 1.82 (s, 3H, CH₃), 3.31 (s, 3H, CH₃), 3.77 (q, 2H, J=7.0, 14.0 Hz, CH₂) 7.56 (d, 1H, J=8.0 Hz, Ph-H), 7.65 (t, 1H, J=8 Hz, Ph-H), 7.88 (s, 1H, Ph-H) and 8.06 (d, 1H, J=8 Hz, Ph-H); MS (ESI⁺) m/z 205.91 [M], C₁₂H₁₅NO₂ requires 205.25.

This material (0.23 g, 1.13 mmol), redissolved in MeCN (2 mL), was treated with N,N-dimethylformamide dimethylacetal (150 μL, 1.12 mmol) at 180° C. for 10 min in a microwave reactor (SmithCreator, Personal Chemistry Ltd.). The solvent was evaporated and the residue was filtered and washed with EtOAc/PE (1:3) to afford N-[3-(3-dimethylamino-acryloyl)-phenyl]-N-ethyl-acetamide as an orange solid (0.30 g, 100%). ¹H-NMR (CD₃OD): δ 1.11 (t, 3H, J=7.0 Hz, CH₃), 1.82 (s, 3H, CH₃), 2.04 (s, 6H, CH₃), 3.76 (q, 2H, J=7.0, 14.0 Hz, CH₂), 5.87 (d, 1H, J=12.0 Hz, CH), 7.39 (d, 1H, J=8.0 Hz, Ph-H), 7.55 (t, 1H, J=8.0 Hz, 5-H), 7.76 (s, 1H, Ph-H), 7.89 (d, 1H, J=12.0 Hz, CH), 7.93 (d, 1H, J=8.0 Hz, Ph-H); MS (ESI⁺) m/z 261.32 [M+H]+, C₁₅H₂₀N₂O₂ requires 260.33.

A solution of this material (0.228 g, 0.88 mmol), 4-hydroxy-phenyl guanidine nitrate (0.188 g, 0.88 mmol) and NaOH (35 mg, 0.88 mmol) in MeCN (2 mL) was heated at 190° C. for 15 min in the microwave reactor. The solvent was evaporated and the residue was purified by SiO₂ gel chromatography (EtOAc/PE, 1:1) to afford the title compound as a yellow solid (117 mg, 38%). ¹H-NMR (CD₃OD): δ 1.16 (t, 3H, J=7 Hz, CH₃), 3.35 (s, 3H, CH₃), 3.38 (q, 2H, J=7.0, 14.0 Hz, CH₂), 6.77 (d, 2H, J=9.0 Hz, Ph-H), 7.27 (d, 1H, J=5.0 Hz, pyrimidine-H), 7.42 (d, 1H, J=8.0 Hz, Ph-H), 7.48 (d, 2H, J=9.0 Hz, Ph-H), 7.62 (t, 1H, J=8.0 Hz, Ph-H), 8.6 (s, 1H, Ph-H), 8.14 (d, 1H, J=8.0 Hz, Ph-H), 8.41 (d, 1H, J=5.0 Hz, pyrimidine-H).

### Example 4

### N-{3-[2-(4-Hydroxy-phenylamino)-pyrimidin-4-yl]-phenyl}-acetamide (11)

This compound was obtained by treatment of N-[3-(3-dimethylamino-acryloyl)-phenyl]-acetamide and 4-hydroxy-phenyl guanidine nitrate in MeCN: 98 mg yellow solid (30%). ¹H-NMR (CD₃OD): δ 3.32 (s, 3H, CH₃), 6.79 (d, 2H, J=9.0 Hz, Ph-H), 7.18 (d, 1H, J=5.0 Hz, pyrimidine-H), 7.44 (t, 1H, J=8.0 Hz, Ph-H), 7.50 (d, 2H, J=9.0 Hz, Ph-H), 7.65 (d, 1H, J=9.0 Hz, Ph-H), 7.84 (d, 1H, J=8.0 Hz, Ph-H), 8.35 (s, 1H, Ph-H), 8.37 (d, 1H, J=5.0 Hz, pyrimidine-H).

N-[3-(3-Dimethylamino-acryloyl)-phenyl]-acetamide was prepared by treatment of N-(3-acetyl-phenyl)-acetamide with N,N-dimethylformamide dimethylacetal (93%): ¹H-NMR (CD₃OD): δ 2.14 (s, 6H, CH₃), 2.58 (s, 3H, CH₃), 5.79 (d, 1H, J=12.0 Hz, CH), 7.37 (t, 1H, J=8.0 Hz, Ph-H), 7.58 (d, 1H, J=8.0 Hz, Ph-H), 7.7 (d, J=8.0 Hz, 1H, Ph-H), 7.83 (d, J=12.0 Hz, 1H, CH), 8.02 (s, 1H, 2-H); MS (ESI⁺) m/z 233.20 [M+H]⁺, C₁₃H₁₆N₂O₂ requires 232.28.

### Example 5

### [4-(3-Imidazol-1-ylmethyl-phenyl)-pyrimidin-2-yl]-(3-nitro-phenyl)-amine (29)

A solution of 1-m-tolyl-ethanone (5.0 g, 37.3 mmol) in anh. MeCN (45 mL) was treated with N-bromosuccinimide (6.63 g, 37.3 mmol) and benzoyl peroxide (9.02 g, 37.3 mmol). The reaction mixture was heated at 80° C. for 6 h. On cooling, the mixture was concentrated and the resulting syrup was dissolved in Et₂O and treated with NaHCO₃. The ethereal layer was washed with brine and dried on MgSO₄. The solvent was evaporated and the resulting residue was purified by SiO₂ gel chromatography (heptane[EtOAc 12:1-3:1) to afford 1-(3-bromomethyl-phenyl)-ethanone (5.5 g, 69%). ¹H-NMR (CDCl₃): δ 2.54 (s, 3H, CH₃), 4.45 (s, 2H, CH₂), 7.38 (t, 1H, J=8.0 Hz, Ph-H), 7.52 (d, 1H, J=8.0 Hz, Ph-H), 7.81 (d, 1H, J=8.0 Hz, Ph-H), 7.90 (s, 1H, Ph-H).

1H-Imidazole (0.15 g, 2.25 mmol) in anh. DMF (8 mL) was cooled on an ice bath and treated with Cs₂CO₃ (0.67 g, 2.07 mmol). After stirring for 30 min 1-(3-bromomethyl-phenyl)-ethanone (0.4 g, 1.88 mmol) was added. The reaction mixture was warmed to room temperature and was stirred for 20 h. Ice water was added and the mixture was extracted with Et₂O. The combined extracts were washed with brine and dried on MgSO₄. The solvent was evaporated and the residue was purified by SiO₂ gel chromatography using heptane/EtOAc (12:1-3:1) to afford 1-(3-imidazol-1-ylmethyl-phenyl)-ethanone (0.23 g, 60%) as a brown syrup. ¹H-NMR (CDCl₃) δ: 2.57 (s, 3H, CH₃), 5.16 (s, 2H, CH₂), 6.89 (s, 1H, imidazole-H), 7.08 (s, 1H, imidazole-H), 7.30 (d, 1H, J=8.0 Hz, Ph-H), 7.45 (t, 1H, J=8.0 Hz, Ph-H), 7.54 (s, 1H, imidazole-H), 7.77 (s, 1H, Ph-H), 7.89 (d, 1H, J=8.0 Hz, Ph-H).

An aliquot of this material (0.10 g, 0.50 mmol) was treated with N,N-dimethyl formamide dimethylacetal (1 mL, 8.39 mmol) at 100° C. for 7 h. On cooling, the reaction mixture was concentrated and the resulting residue was purified by SiO₂ chromatography using heptane/EtOAc (3:1-1:10) to afford 3-dimethylamino-1-(3-imidazol-1-ylmethyl-phenyl)-propenone as yellow solid (0.11 g, 83%). ¹H-NMR (CDCl₃) δ: 2.88 (s, 3H, CH₃), 3.11 (s, 3H, CH₃), 5.12 (s, 2H, CH₂), 5.61 (d, 1H, J=12.0 Hz, CH), 6.88 (s, 1H, imidazole-H), 7.04 (s, 1H, imidazole-H), 7.15 (d, 1H, J=6.0 Hz, Ph-H), 7.35 (t, 1H, J=7.5 Hz, Ph-H), 7.54 (s, 1H, imidazole-H), 7.71 (s, 1H, Ph-H), 7.75 (m, 2H, Ph-H and CH).

A mixture of the latter compound (0.10 g, 0.39 mmol), 3-nitro-phenyl guanidine nitrate (0.11 g, 0.43 mmol), and NaOH (0.019 g, 0.47 mmol) in 2-methoxylethanol (4 mL) was heated at 125° C. for 20 h. The solvent was evaporated and the residue was purified by SiO₂ gel chromatography using EtOAc and EtOAc/MeOH (10:1) to afford the title compound as a yellow solid (0.079 g, 55%). Anal. RP-HPLC: t_{R}=17 min (0-60% MeCN, purity>95%). ¹H-NMR (DMSO-d₆): δ 5.32 (s, 2H, CH₂), 6.91 (s, 1H, imidazole-H), 7.23 (s, 1H, imidazole-H), 7.41 (d, 1H, J=8.0 Hz, Ph-H), 7.51 (d, J=5.5 Hz, pyrimidine-H), 7.54 (t, 1H, J=8.0 Hz, Ph-H), 7.59 (t, 1H, J=8.0 Hz, Ph-H), 7.81 (m, 2H, Ph-H), 8.05 (d, 1H, J=8.0 Hz, Ph-H), 8.14 (d, 1H, J=8.0 Hz, Ph-H), 8.18 (s, 1H, Ph-H), 8.65 (d, 1H, J=5.5 Hz, pyrimidine-H), 9.14 (s, 1H, imidazole-H), 10.27 (s, 1H, NH). ¹³C-NMR (DMSO-d₆): δ 60.4, 109.8, 113.1, 116.3, 120.3, 125.3, 126.8, 127.2, 129.5, 130.1, 130.5, 130.6, 137.5, 138.1, 139.4, 142.6, 148.9, 160.2, 160.4, 163.9. MS (ESI⁺) m/z 373.2 [M+H]⁺, C₂₀H₁₆N₆O₂ requires 372.38.

### Example 6

The following compounds were prepared in a similar manner to that described in Example 5 above:

### (3-Nitro-phenyl)-[4-(3-[1,2,4]triazol-1-ylmethyl-phenyl)-pyrimidin-2-yl]-amine (30)

By treatment of 3-dimethylamino-1-(3-[1,2,4]triazol-1-ylmethyl-phenyl)-propenone and 3-nitro-phenyl guanidine nitrate. Yellow solid (50%). Anal. RP-HPLC: t_{R}=17 min (0-60% MeCN, purity>95%). ¹H-NMR (DMSO-d₆): δ 5.54 (s, 2H, CH₂), 7.43 (d, 1H, J=8.0 Hz, Ph-H), 7.51 (d, 1H, J=5.0 Hz, pyrimidine-H), 7.54 (t, 1H, J=8.0 Hz, Ph-H), 7.58 (t, 1H, J=8.0 Hz, Ph-H), 7.81 (d, 1H, J=8.0 Hz, Ph-H), 7.98 (s, 1H, Ph-H), 8.07 (d, 1H, J=8.0 Hz, Ph-H), 8.15 (d, 1H, J=8.0 Hz, Ph-H), 8.21 (s, 1H, Ph-H), 8.65 (d, 1H, J=5.0 Hz, pyrimidine-H), 8.70 (s, 1H, triazole-H), 9.11 (s, 1H, triazole-H), 10.27 (s, 1H, NH). MS (ESI⁺) m/z 374.4 [M+H]+, C₁₉H₁₅N₇O₂ requires 373.37.

### 3-dimethylamino-]-(3-[1,2,4]triazol-1-ylmethyl-phenyl)-propenone

¹H-NMR (DMSO-d₆): δ 2.89 (s, 3H, CH₃), 3.12 (s, 3H, CH₃), 5.45 (s, 2H, CH₂), 5.76 (d, 1H, J=12.5 Hz, CH), 7.35 (d, 1H, J=8.0 Hz, Ph-H), 7.40 (t, 1H, J=8.0 Hz, Ph-H), 7.70 (d, 1H, J=12.5 Hz, CH), 7.79 (s, 1H, Ph-H), 7.82 (d, 1H, J=8.0 Hz, Ph-H), 7.97 (s, 1H, triazole-H), 8.67 (s, 1H, triazole-H).

### 1-(3-[1,2,4]triazol-1-ylmethyl-phenyl)-ethanone

¹H-NMR (CDCl₃): δ 2.58 (s, 3H, CH₃), 5.39 (s, 2H, CH₂), 7.45 (d, 1H, J=7.5 Hz, Ph-H), 7.47 (t, 1H, J=7.5 Hz, Ph-H), 7.87 (s, 1H, Ph-H), 7.92 (d, 1H, J=7.5 Hz, Ph-H), 7.97 (s, 1H, triazole-H), 8.11 (s, 1H, triazole-H).

### {4-[3-(Benzylamino-methyl)-phenyl]-pyrimidin-2-yl}-(6-chloro-pyridin-3-yl)-amine (48)

By treatment of 1-[3-(benzylamino-methyl)-phenyl]-3-dimethylamino-propenone and N-(6-chloro-pyridin-3-yl)-guanidine nitrate, which was prepared by condensation of 5-amino-2-chloropyridine and aqueous cyanamide solution in the presence of HNO₃. Yellow solid (35%). Anal. RP-HPLC: t_{R=}24 min (0-60% MeCN, purity >95%). ¹H-NMR (CDCl₃): δ 4.30 (m, 2H, CH₂), 4.44 (m, 2H, CH₂), 7.13 (m, 3H, Ph-H), 7.24-7.28 (m, 4H, Ph-H), 7.37 (d, 1H, J=8.0 Hz, Ph-H), 7.44 (m, 2H, pyrimidine-H and Ph-H), 7.84 (s, 1H, Ph-H), 7.90 (d, 1H, J=8.0 Hz, Ph-H), 8.18 (m, 1H, Ph-H), 8.45 (m, 2H, pyrimidine-H and NH), 8.58 (m, 1H, NH). MS (ESI⁺) m/z 402.5 [M+H]+, C₂₃H₂₀ClN₅ requires 401.89.

### 1-[3-(Benzylamino-methyl)-phenyl]-3-dimethylamino-propenone

¹H-NMR (CDCl₃): δ 2.94 (s, 3H, CH₃), 3.14 (s, 3H, CH₃), 3.79 (s, 2H, CH₂), 3.83 (s, 2H, CH₂), 5.70 (d, 1H, J=12.5 Hz, CH), 7.21 (t, 1H, J=7.5 Hz, Ph-H), 7.29 (d, 2H, J=7.0 Hz, Ph-H), 7.34 (m, 3H, Ph-H), 7.76 (d, 1H, J=7.5 Hz, Ph-H), 7.80 (d, 1H, J=12.5 Hz, CH), 7.87 (s, 1H, Ph-H).

### 1-[3-(Benzylamino-methyl)-phenyl]-ethanone

¹H-NMR (CDCl₃): δ 2.61 (s, 3H, CH₃), 3.82 (s, 2H, CH₂), 3.86 (s, 2H, CH₂), 7.26 (m, 4H, Ph-H), 7.34 (m, 1H, Ph-H), 7.42 (t, 1H, J=7.5 Hz, Ph-H), 7.58 (d, 1H, J=7.5 Hz, Ph-H), 7.85 (d, 1H, J=7.5 Hz, Ph-H), 7.94 (s, 1H, Ph-H).

### 3-{4-[3-(Benzylamino-methyl)-phenyl]-pyrimidin-2-ylamino}-phenol (28)

By treatment of 1-[3-(benzylamino-methyl)-phenyl]-3-dimethylamino-propenone and N-(3-hydroxy-phenyl)-guanidine nitrate. Yellow solid (10%). Anal. RP-HPLC: t_{R}=11 min (10-70% MeCN, purity>95%). ¹H-NMR (DMSO-d₆): δ 4.58 (s, 2H, CH₂), 6.37 (d, 2H, J=7.5 Hz, Ph-H), 7.05 (t, 1H, J=8.0 Hz, Ph-H), 7.34 (m, 2H, Ph-H), 7.47 (m, 2H, pyrimidine-H and Ph-H), 8.03 (m, 1H, Ph-H), 8.11 (s, 1H, Ph-H), 8.52 (d, 1H, J=5.5 Hz, pyrimidine-H), 9.54 (s, 1H, NH).

### (6-Methoxy-pyridin-3-yl)-[4-(3-[1,2,4]triazol-1-ylmethyl-phenyl)-pyrimidin-2-yl]-amine (50)

By treatment of 3-dimethylamino-1-(3-[1,2,4]triazol-1-ylmethyl-phenyl)-propenone and N-(6-methoxy-pyridin-3-yl)-guanidine nitrate. Yellow solid (54%). Anal. RP-HPLC: t_{R}=12 min (10-70% MeCN, purity>95%). ¹H-NMR (CD₃OD): δ 3.91 (s, 3H, CH₃), 5.54 (s, 2H, CH₂), 6.25(d, 1H, J=9.0 Hz, Ph-H), 7.26 (d, 1H, J=5.0 Hz, pyrimidine-H), 7.45 (d, 1H, J=7.0 Hz, Ph-H), 7.51 (t, 1H, J=7.0, 8.0 Hz, Ph-H), 8.03 (m, 2H, triazole-H and Ar—H), 8.07 (m, 2H, Ar—H), 8.42 (d, 1H, J=5.0 Hz, pyrimidine-H), 8.50 (d, 1H, J=5.0 Hz, Ar—H), 8.61 (s, 1H, triazoe-H). MS (ESI⁺) m/z 360.3 [M+H]⁺, C₁₉H₁₇N₇O requires 359.38.

### (4-Morpholin-4-yl-phenyl)-[4-(3-[1,2,4]triazol-1-ylmethyl-phenyl)-pyrimidin-2-yl]-amine (32)

By treatment of 3-dimethylamino-1-(3-[1,2,4]triazol-1-ylmethyl-phenyl)-propenone and N-(4-morpholin-4-yl-phenyl)-guanidine nitrate. Yellow solid (44%). Anal. RP-HPLC: t_{R}=11 min (10-70% MeCN, purity>95%). ¹H-NMR (CD₃OD): δ 3.12 (t, 4H, J=4.0, 5.0 Hz, CH₂), 3.85 (t, 4H, J=4.5, 5.0 Hz, CH₂), 5.53 (s, 2H, CH₂), 7.00 (d, 2H, J=9.0 Hz, Ph-H), 7.22 (d, 1H, J=5.0 Hz, pyrimidine-H), 7.46 (d, 1H, J=7.0 Hz, Ph-H), 7.51 (t, 1H, J=7.0, 8.0 Hz, Ph-H), 7.59 (d, 1H, Ph-H), 8.02 (s, 1H, triazole-H), 8.07 (m, 1H, Ph-H), 8.39 (d, 1H, J=5.0 Hz, pyrimidine-H), 8.61 (s, 1H, triazol-H). MS (ESI⁺) m/z 414.4 [M+H]+, C₂₃H₂₃N₇O requires 413.48.

### 4-[4-(3-[1,2,4]Triazol-1-ylmethyl-phenyl)-pyrimidin-2-ylamino]-phenol (33)

By treatment of 3-dimethylamino-1-(3-[1,2,4]triazol-1-ylmethyl-phenyl)-propenone and 4-hydroxy-phenyl guanidine nitrate. Yellow solid (30%). Anal. RP-HPLC: t_{R}=9.5 min (10-70% MeCN, purity>95%). ¹H-NMR (DMSO-d₆): δ 5.51 (s, 2H, CH₂), 6.42 (d, 2H, J=8.5 Hz, Ph-H), 7.26 (d, 1H, J=5.0 Hz, pyrimidine-H), 7.43 (d, 1H, J=7.0 Hz, Ph-H), 7.52 (m, 3H, Ph-H), 8.00 (s, 1H, triazole-H), 8.04 (m, 2H, Ph-H), 8.46 (d, 1H, J=5.0 Hz, pyrimidine-H), 8.71 (s, 1H, triazole-H). 9.35 (br. s, 1H, NH). ¹³C-NMR (DMSO-d₆): δ 48.50, 53.50, 107.80, 115.70, 121.70, 121.80, 126.90, 127.10, 127.20, 129.90, 130.90, 130.95, 132.60, 137.60, 137.90, 145.00, 152.50, 153.00, 160.95. MS (ESI⁺) m/z 345.4 [M+H]+, C₁₉H₁₆N₆O requires 344.37.

### 3-[4-(3-[1,2,4]Triazol-1-ylmethyl-phenyl)-pyrimidin-2-ylamino]-phenol (34)

By treatment of 3-dimethylamino-1-(3-[1,2,4]triazol-1-ylmethyl-phenyl)-propenone and 3-hydroxy-phenyl guanidine nitrate. Yellow solid (32%). Anal. RP-HPLC: t_{R}=10.8 min (10-70% MeCN, purity>95%). ¹H-NMR (DMSO-d₆): δ 5.51 (s, 2H, CH₂), 6.38 (d, 1H, J=8.0 Hz, Ph-H), 7.07 (t, 1H, J=8.0 Hz, Ph-H), 7.24 (d, 1H, J=8.0 Hz, Ph-H), 7.31 (s, 1H, Ph-H), 7.34 (d, 1H, J=5.0 Hz, pyrimidine-H), 7.43 (d, 1H, J=7.5 Hz, Ph-H), 7.53 (t, 2H, J=7.5, 8.0 Hz, Ph-H), 8.00 (s, 1H, triazole-H), 8.09 (m, 1H, Ph-H), 8.53 (d, 1H, J=5.0 Hz, pyrimidine-H), 8.72 (s, 1H, triazole-H), 9.55 (br. s, 1H, NH). ¹³C-NMR (DMSO-d₆): δ 49.30, 52.70, 106.70, 108.60, 109.40, 110.60, 127.10, 129.80, 129.90, 131.00, 137.60, 137.80, 142.20, 145.00, 152.50, 158.20, 159.71, 160.90, 163.90. MS (ESI⁺) m/z 345.3 [M+H]⁺, C₁₉H₁₆N₆O requires 344.37.

### (3-Methoxy-phenyl)-[4-(3-[1,2,4]triazol-1-ylmethyl-phenyl)-pyrimidin-2-yl]-amine (35)

By treatment of 3-dimethylamino-1-(3-[1,2,4]triazol-1-ylmethyl-phenyl)-propenone and 3-methoxy-phenyl guanidine nitrate. Yellow solid (47%). Anal. RP-HPLC: t_{R}=14.5 min (10-70% MeCN, purity>95%). ¹H-NMR (DMSO-d₆): δ 3.75 (s, 3H, CH₃), 5.51 (s, 2H, CH₂), 6.54 (d, 1H, J=8.0 Hz, Ph-H), 7.20 (t, 1H, J=7.5, 8.0 Hz, Ph-H), 7.32 (m, 2H, pyrimidine-H and Ph-H), 7.44 (d, 1H, J=8.0 Hz, Ph-H), 7.53 (m, 2H, Ph-H), 7.99 (s, 1H, triazole-H), 8.08 (m, 2H, Ph-H), 8.56 (d, 1H, J=5.5 Hz, pyrimidine-H), 8.71 (s, 1H, triazole-H), 9.67 (br. s, 1H, NH). ¹³C-NMR (DMSO-d₆): δ 2.70, 55.60, 105.30, 107.50, 108.80, 109.90, 111.90, 127.10, 127.20, 129.90, 131.10, 137.70, 137.80, 142.40, 145.00, 152.50, 159.80, 160.20, 160.80, 163.80. MS (ES1+) m/z 359.4 [M+H]⁺, C₂₀H₁₈N₆O requires 358.40.

### 3-[4-(3-[1,2,4]triazol-1-ylmethyl-phenyl)-pyrimidin-2-ylamino]-benzonitrile (36)

By treatment of 3-dimethylamino-1-(3-[1,2,4]triazol-1-ylmethyl-phenyl)-propenone and N-(3-cyano-phenyl)-guanidine nitrate. Yellow solid (47%). Anal. RP-HPLC: t_{R}=15.6 min (10-70% MeCN, purity>95%). ¹H-NMR (DMSO-d₆): δ 5.52 (s, 2H, CH₂), 7.39 (d, 1H, J=7.5 Hz, Ph-H), 7.46 (m, 2H, pyrimidine-H and Ph-H), 7.54 (m, 2H, Ph-H), 8.01 (s, 1H, triazole-H), 8.09 (m, 3H, Ph-H), 8.31 (s, ¹H, Ph-H), 8.63 (d, 1H, J=5.0 Hz, pyrimidine-H), 8.73 (s, 1H, triazole-H), 10.09 (br. s, 1H, NH). ¹³C-NMR (DMSO-d₆): δ 52.70, 109.70, 112.10, 119.80, 121.90, 123.80, 125.30, 127.20, 130.10, 130.70, 131.20, 137.50, 137.70, 142.10, 145.00, 452.50, 159.00, 159.90, 160.40, 164.10. MS (ESI⁺) m/z 354.3 [M+H]⁺, C₂₀H₁₅N₇ requires 353.38.

### Example 7

[4-(4-Chloro-3-[1,2,4]triazol-1-ylmethyl-phenyl)-pyrimidin-2-yl]-(3-nitro-phenyl)-amine (47)

By treatment of 3-dimethylamino-1-(4-chloro-3-[1,2,4]triazol-1-yl-methyl-phenyl)-propenone with 3-nitro-phenyl guanidine nitrate. Yellow solid. Anal. RP-HPLC: t_{R}=19.9 min (10-70% MeCN, purity 95%). ¹H-NMR (DMSO-d₆) δ: 5.61 (s, 2H, CH₂), 7.50 (d, 1H, J=5.0 Hz, pyrimidinyl-H), 7.61 (t, 1H, J=8.5 Hz, Ph-H), 7.70 (d, 1H, J=8.5 Hz, Ph-H), 7.83 (d, 1H, J=8.5Hz, Ph-H), 7.97 (s, 1H, Ph-H), 8.07 (d, J=8.5Hz, Ph-H), 8.19 (m, 2H, Ph-H and NH), 8.67 (d, 1H, J=5.0Hz, pyrimidinyl-H), 8.70 (s, 1H, Ar—H), 9.01 (1 s, 1H, Ar—H), 10.31 (sbr, 1H, NH). MS (ESI⁺) m/z 408.12 [M+H]+, C₁₉H₁₄ClN₇O₂ requires 407.81.

### Example 8

### (6-Methoxy-pyridin-3-yl)-{4-[3-(4-methyl-piperazin-1-ylmethyl)-phenyl]-pyrimidin-2-yl}-amine (58)

By treatment of 3-dimethylamino-1-(3-(4-methyl-piperazinyl-1-yl-methyl-phenyl)-propenone with 6-methoxy-pyridin-3-yl guanidine nitrate. Orange solid. Anal. RP-HPLC: t_{R}=8.9 min (10-70% MeCN, purity 100%). ¹H-NMR (CD₃OD) δ: 2.91 (s, 3H, CH₃), 3.07 (m, 4H, CH₂33 2), 3.41 (m, 4H, CH₂), 3.99 (s, 3H, OCH₃), 4.02 (s, 2H, CH₂), 7.05 (d, 1H, J=8.0 Hz, Ph-H), 7.41 (d, 1H, J=5.0 Hz, pyrimidinyl-H), 7.59 (m, 2H, Ph-H and Ar—H), 8.15 (m, 2H, Ph-H and Ar—H), 8.20 (s, 1H, Ph-H), 8.48 (d, 1H, J=5.0 Hz, pyrimidinyl-H), 8.73 (s, 1H, Ar—H). MS (ESI⁺) m/z 391.25 [M+H]⁺, C₂₂H₂₆N₆O₂ requires 390.48.

### Example 9

### [4-(3-Imidazol-1-ylmethyl-phenyl)-pyrimidin-2-yl]-(6-methoxy-pyridin-3-yl)-amine (59)

By treatment of 3-dimethylamino-1-(3-(imidazol-1-yl-methyl-phenyl)-propenone with 6-methoxy-pyridin-3-yl guanidine nitrate. Yellow solid. Anal. RP-HPLC: t_{R}=9.8 min (10-70% MeCN, purity 100%). ¹H-NMR (CD₃OD) δ: 3.97 (s, 3H, OCH₃), 5.57 (s, 2H, CH₂), 6.92 (d, 1H, J=8.5 Hz, Ph-H), 7.36 (d, 1H, J=5.5 Hz, pyrimidinyl-H), 7.59 (m, 3H, Ph-H and Ar—H), 7.69 (s, 1H, Ar—H), 8.05 (m, 1H, Ph-H), 8.19 (m, 2H, Ph-H and Ar—H), 8.48 (d, 1H, J=5.5 Hz, pyrimidinyl-H), 8.64 (m, 1H, Ar—H), 9.10 (s, 1H, Ar—H). MS (ESI⁺) m/z 359.06 [M+H]⁺, C₂₀H₁₈N₆O requires 358.40.

### Example 10

### [4-(3-Dimethylaminomethyl-phenyl)-pyrimidin-2-yl]-(3-nitro-phenyl)-amine (71)

By treatment of 3-dimethylamino-1-(3-N,N-dimethylamino-methyl-phenyl)-propenone with 3-nitro-phenyl guanidine nitrate. Yellow solid. Anal. RP-HPLC: t_{R}=13.5 min (10-70% MeCN, purity 100%). ¹H-NMR (CD₃OD) δ: 2.94 (s, 6H, CH₃), 4.49 (s, 2H, CH₂), 7.50 (d, 1H, J=5.5 Hz, pyrimidinyl-H), 7.53 (t, 1H, J=8.5 Hz, Ph-H), 7.70 (d, 2H, Ph-H), 7.74 (d, 1H, J=8.5Hz, Ph-H), 7.88 (1H, d, J=8.5 Hz, Ph-H), 8.32 (s, 1H, Ph-H), 8.61 (m, 2H, Ph-H and pyrimidinyl-H), 9.60 (s, 1H, Ph-H), 10.31 (sbr, 1H, NH). MS (ESI⁺) m/z 350.43 [M+H]⁺, C₁₉H₁₉N₅O₂ requires 349.39.

### Example 11

### 3-[4-(4-Methoxy-phenyl)-pyrimidin-2-ylamino]-phenol (76)

By treatment of 3-dimethylamino-1-(4-methoxyphenyl)-propenone with 3-hydroxy-phenyl guanidine nitrate. Brown solid. Anal. RP-HPLC: t_{R}=13.9 min (10-70% MeCN, purity 100%). ¹H-NMR (CDCl₃) δ: 3.89 (s, 3H, CH₃), 6.55 (1H, d, J=8.5 Hz, Ph-H), 7.01 (m, 2H, Ph-H), 7.11 (d, 1H, J=5.5 Hz, pyrimidinyl-H), 7.13 (s, 1H, Ph-H), 7.21 (t, 1H, J=8.5 Hz, Ph-H), 7.40 (sbr, 1H, OH), 7.46 (m, 1H, Ph-H), 8.05 (d, 2H, J=8.5 Hz, Ph-H), 8.39 (1H, d, J=5.5 Hz, pyrimidinyl-H). MS (ESI⁺) m/z 294.41 [M+H]⁺, C₁₇H₁₅N₃O₂ requires 293.32.

### Example 12

### (1-{3-[2-(3-Nitro-phenylamino)-pyrimidin-4-yl]-benzyl}-piperidin-2-yl)-methanol (79)

By treatment of 3-dimethylamino-1-[3-(2-hydroxymethyl-piperidin-1-ylmethyl)-phenyl]-propenone with 3-nitro-phenyl guanidine nitrate. Yellow solid. Anal. RP-HPLC: t_{R}=13.9 min (10-70% MeCN, purity 100%). ¹H-NMR (CDCl₃) δ: 1.42 (m, 2H, CH₂), 1.60 (m, 1H, CH₂), 1.73 (m, 3H, CH₂), 2.28 (m, 1H, CH₂), 2.60 (m, 1H, CH₂), 2.94 (m, 1H, CH₂), 3.52 (m, 1H, CH₂), 3.61 (dd, 1H, J=4.5 Hz, CH₂), 3.90 (dd, 1H, J=4.5 Hz, CH₂), 4.23 (d, 1H, J=13.0 Hz, CH₂), 7.29 (d, 1H, J=5.0 Hz, pyrimidinyl-H), 7.47-7.55 (m, 4H, Ph-H), 7.67 (s, 1H, Ph-H), 7.74 (d, 1H, J=8.5 Hz, Ph-H), 7.89 (1H, d, J=8.0 Hz, Ph-H), 7.99 (m, 1H, Ph-H), 8.14 (s, 1H, Ph-H), 8.52 (d, 1H, J=5.0 Hz, pyrimidnyl-H), 9.13 (sbr, 1H, NH). ¹³C-NMR (DMSO-d₆) δ: 23.60, 25.50, 27.46, 28.93, 52.18, 58.29, 63.35, 109.79, 113.07, 116.27, 125.31, 126.22, 127.93, 129.47, 130.43, 132.24, 136.86, 141.26, 142.66, 148.86, 159.96, 160.44, 164.53. MS (ESI⁺) m/z 420.47 [M+H]⁺, C₂₃H₂₅N₅O₃ requires 419.48.

### Example 13

### 3-[4-(3-Dimethylaminomethyl-phenyl)-pyrimidin-2-ylamino]-phenol (80)

By treatment of 3-dimethylamino-1-(3-dimethylaminomethyl-phenyl)-propenone e with 3-hydroxyphenyl guanidine nitrate. Brown solid. Anal. RP-HPLC: t_{R}=8.9 min (10-70% MeCN, purity 95%). ¹H-NMR (CD₃OD) δ: 2.37 (s, 6H, CH₃×2), 3.63 (s, 2H, CH₂), 6.55 (dd, J=2.0, 8.0 Hz, Ph-H), 6.73 (m, 1H, Ph-H), 7.16 (m, 2H, pyrimidinyl-H and Ph-H), 7.36 (s, 1H, Ph-H), 7.43 (t, 1H, J=7.5 Hz, Ph-H), 7.87 (d, 2H, J=7.0 Hz, Ph-H), 8.06 (s, 1H, OH), 8.42 (d, 1H, J=4.5Hz, pyrimidinyl-H), 8.45 (s, 1H, Ph-H). MS (ESI⁺) m/z 321.51 [M+H]⁺, C₁₉H₂₀N₄O requires 320.39.

### Example 14

### 4-[4-(3-Dimethylaminomethyl-phenyl)-pyrimidin-2-ylamino]-phenol (81)

By treatment of 3-dimethylamino-1-(3-dimethylaminomethyl-phenyl)-propenone with 4-hydroxyphenyl guanidine nitrate. Brown solid. Anal. RP-HPLC: t_{R}=7.6 min (10-70% MeCN, purity 100%). ¹H-NMR (CD₃OD) δ: 2.36 (s, 6H, CH₃×2), 3.62 (s, 2H, CH₂), 6.81 (dd, J=9.0 Hz, Ph-H), 6.97 (m, 1H, Ph-H), 7.11 (d, 1H, J=5.5 Hz, pyrimidinyl-H), 7.47 (m, 2H, Ph-H), 7.96 (sbr, 1H, OH), 8.06 (s, 1H, Ph-H), 8.40 (d, 1H, J=5.5 Hz, pyrimidinyl-H). MS (ESI⁺) m/z 321.51 [M+H]⁺, C₁₉H₂₀N₄O requires 320.39.

### Example 15

### [4-(3-Dimethylaminomethyl-phenyl)-pyrimidin-2-yl]-(4-morpholin-4-yl-phenyl)-amine (82)

By treatment of 3-dimethylamino-1-(3-dimethylaminomethyl-phenyl)-propenone with 4-morpholino-phenyl guanidine nitrate. Yellow solid. Anal. RP-HPLC: t_{R}=8.3 min (10-70% MeCN, purity 98%). ¹H-NMR (CD₃OD) δ: 2.31 (s, 6H, CH₃×2), 3.14 (m, 4H, CH₂), 3.55 (s, 2H, CH₂), 3.89 (m, 4H, CH₂), 6.95 (d, 2H, J=9.0 Hz, Ph-H), 7.14 (m, 2H, pyrimidinyl-H and Ph-H), 7.45 (d, 2H, J=4.5 Hz, Ph-H), 7.59 (d, 2H, J=9.0 Hz, Ph-H), 7.97 (sbr, 1H, OH), 8.01 (s, 1H, Ph-H), 8.43 (d, 1H, J=5.0 Hz, pyrimidinyl-H). MS (ESI⁺) m/z 390.55 [M+H]⁺, C₂₃H₂₇N₅O requires 389.49.

### Example 16

### [4-(3-Dimethylaminomethyl-phenyl)-pyrimidin-2-yl]-(6-methoxy-pyridin-3-yl)-amine (83)

By treatment of 3-dimethylamino-1-(3-dimethylaminomethyl-phenyl)-propenone with 6-methoxy-pyridin-3-yl guanidine nitrate. Yellow solid. Anal. RP-HPLC: t_{R}=9.8 min (10-70% MeCN, purity 100%). ¹H-NMR (CD₃OD) δ: 2.30 (s, 6H, CH₃), 3.54 (s, 2H, CH₂), 3.95 (s, 3H, OCH₃), 6.78 (d, 1H, J=9.5 Hz, Ph-H), 7.18 (d, 1H, J=5.5 Hz, pyrimidinyl-H), 7.21 (s, 1H, Ph-H/Ar—H), 7.45 (m, 1H, Ar—H), 7.96 (m, 1H, Ar—H), 8.00 (m, 1H, Ph-H), 8.04 (dd, 1H, J=2.5, 8.5 Hz, Ph-H), 8.35 (d, 1H, J=2.5 Hz, Ar—H), 8.43 (d, 1H, J=5.5 Hz, pyrimidinyl-H). MS (ESI⁺) m/z 336.51 [M+H]⁺, C₁₉H₂₁N₅O requires 335.40.

### Example 17

### [4-(3-Diethylaminomethyl-phenyl)-pyrimidin-2-yl]-(3-nitro-phenyl)-amine (84)

By treatment of 1-(3-diethylaminomethyl-phenyl)-3-dimethylamino-propenone with 3-nitro-phenyl guanidine nitrate. Yellow solid. Anal. RP-HPLC: t_{R}=14.0 min (10-70% MeCN, purity 100%). ¹H-NMR (DMSO-d₆) δ: 1.02 (t, J=6.5 Hz, 6H, CH₃), 2.59 (m, 4H, CH₂), 3.73 (s, 2H, CH₂), 7.53-7.60 (m, 2H, Ph-H and pyrimidinyl-H), 7.81 (m, 1H, J=8.5 Hz, Ph-H), 7.70 (d, 2H, Ph-H), 7.74 (d, 1H, J=8.5 Hz, Ph-H), 7.88 (m, 1H, Ph-H), 8.09 (m, 1H, Ph-H), 8.20 (s, 1H, Ph-H), 8.65 (d, 1H, J=5.0 Hz, pyrimidinyl-H), 9.16 (m, 1H, Ph-H), 10.26 (sbr, 1H, NH). ¹³C-NMR (DMSO-d₆) δ: 12.03, 31.39, 46.85, 57.35, 109.74, 113.09, 116.33, 125.34, 126.41, 127.97, 129.56, 130.45, 132.27, 136.93, 142.66, 148.86, 160.06, 160.44, 164.41. MS (ESI⁺) m/z 378.40 [M+H]⁺, C₂₁H₂₃N₅O₂ requires 377.44.

### Example 18

### N-Methyl-3-nitro-N-{3-[2-(3-nitro-phenylamino)-pyrimidin-4-yl]-benzyl}-benzene-sulfonamide (85)

By treatment of N-[3-(3-dimethylamino-acryloyl)-benzyl]-I-methyl-3-nitro-benzene-sulfonamide with 3-nitro-phenyl guanidine nitrate. Yellow solid. Anal. RP-HPLC: t_{R}=23.5 min (10-70% MeCN, purity 90%). ¹H-NMR (DMSO-d₆) δ: 2.75 (s, 3H, CH₃), 4.42 (s, 2H, CH₂), 7.31 (d, 1H, J=5.0 Hz, pyrimidinyl-H), 7.49 (t, 1H, J=8.5 Hz, Ph-H), 7.53-7.62 (m, 3H, Ph-H), 7.83(d, 1H, J=7.5 Hz, Ph-H), 7.88 (d, 1H, J=8.0 Hz, Ph-H), 8.00 (d, 1H, J=7.5 Hz, Ph-H), 8.21 (d, 1H, J=7.5 Hz, Ph-H), 8.25 (s, 1H, Ph-H), 8.50 (d, 1H, J=8.0 Hz, Ph-H), 8.54 (d, 1H, J=5.0 Hz, pyrimidinyl-H), 8.70 (m, 1H, Ph-H), 9.29 (s, 1H, Ph-H). MS (ESI⁺) m/z 521.33 [M+H]⁺, C₂₄H₂₀N₆O₆S requires 520.52.

### Example 19

### (3-Nitro-phenyl)-{4-[3-(2-phenylaminomethyl-pyrrolidin-1-ylmethyl)-phenyl]-pyrimidin-2-yl}-amine (86)

By treatment of 3-dimethylamino-1-[3-(2-phenylaminomethyl-pyrrolidin-1-ylmethyl)-phenyl]-propenone with 3-nitro-phenyl guanidine nitrate. Yellow solid. Anal. RP-HPLC: t_{R}=17.8 min (10-70% MeCN, purity 93%). ¹H-NMR (CDCl₃) δ: 1.29 (m, 1H, CH₂), 1.75 (m, 2H, CH₂), 1.84 (m, 1H, CH₂), 1.99 (m, 1H, CH₂), 2.33 (m, 1H, CH₂), 2.91 (m, 1H, CH₂), 3.04 (m, 1H, CH₂), 3.22 (m, 1H, CH₂), 3.47 (m, 1H, CH₂), 4.08 (m, 1H, CH₂), 6.60 (d, 2H, J=8.0 Hz, Ph-H), 6.67 (d, 1H, J=7.0 Hz, Ph-H), 7.13 (t, 2H, J=8.5 Hz, Ph-H), 7.25 (d, 1H, J=5.5 Hz, pyrimidinyl-H), 7.45-7.52 (m, 4H, Ph-H), 7.74 (m, 1H, Ph-H), 7.88 (d, 1H, J=8.5 Hz, Ph-H), 7.99 (1H, d, J=9.0 Hz, Ph-H), 8.11 (s, 1H, Ph-H), 8.52 (d, 1H, J=5.5 Hz, pyrimidnyl-H), 9.12 (s, 1H, Ph-H). MS (ESI⁺) m/z 482.50 [M+H]⁺, C₂₈H₂₈N₆O₂ requires 480.56.

### Example 20

### 1-{3-[2-(3-Nitro-phenylamino)-pyrimidin-4-yl]-benzyl}-piperidine-3-carboxylic acid amide (99)

By treatment of 1-[3-(3-dimethylamino-acryloyl)-benzyl]-piperidine-3-carboxylic acid amide with 3-nitro-phenyl guanidine nitrate. Yellow solid. Anal. RP-HPLC: t_{R}=17.8 min (10-70% MeCN, purity 87%). MS (ESI⁺) m/z 433.48 [M+H]⁺, C₂₃H₂₄N₆O₃ requires 432.48.

### Example 21

### 2-(1-{3-[2-(3-Nitro-phenylamino)-pyrimidin-4-yl]-benzyl}-piperidin-3-yl)-ethanol (100)

By treatment of 3-dimethylamino-1-{3-[3-(2-hydroxy-ethyl)-piperidin-1-ylmethyl]-phenyl}-propenone with 3-nitro-phenyl guanidine nitrate. Brown solid. Anal. RP-HPLC: t_{R}=14.3 min (10-70% MeCN, purity 99%). ¹H-NMR (CDCl₃) δ: 1.40 (m, 1H, CH₂), 1.48 (m, 2H, CH₂), 1.56 (m, 1H, CH₂), 1.72 (m, 2H, CH₂), 1.81 (m, 1H, CH₂), 2.14 (m, 1H, CH₂), 2.23 (m, 1H, CH₂), 2.60 (m, 1H, CH₂), 2.85 (m, 1H, CH₂), 3.33 (m, 1H, CH₂), 3.52 (d, 1H, J=13.5 Hz, CH₂), 3.66 (m, 1H, CH₂), 4.14 (d, 1H, J=13.5 Hz, CH₂), 7.38 (d, 1H, J=5.5 Hz, pyrimidinyl-H), 7.49 (m, 3H, Ph-H), 7.81 (d, 1H, J=8.5 Hz, Ph-H), 7.87(m, 1H, J=8.5 Hz, Ph-H), 8.08 (m, 1H, J=8.0 Hz, Ph-H), 8.21 (m, 1H, Ph-H), 8.51 (d, 1H, J=5.0 Hz, pyrimidinyl-H), 9.22 (s, 1H, Ph-H). MS (ESI⁺) m/z 434.26 [M+H]⁺, C₂₄H₂₇N₅O₃ requires 433.50.

### Example 22

### (1-{3-[2-(4-Morpholin-4-yl-phenylamino)-pyrimidin-4-yl]-benzyl}-piperidin-2-yl)-methanol (101)

By treatment of 3-dimethylamino-1-[3-(2-hydroxymethyl-piperidin-1-ylmethyl)-phenyl]-propenone with 3-nitro-phenyl guanidine nitrate. Brown solid. Anal. RP-HPLC: t_{R}=9.02 min (10-70% MeCN, purity 87%). ¹H-NMR (CD₃OD) δ: 1.35 (m, 1H, CH₂), 1.47-1.59 (m, 3H, CH₂), 1.72-1.81 (m, 2H, CH₂), 2.14 (m, 1H, CH₂), 2.41 (m, 1H, CH₂), 2.84 (m, 1H, CH₂), 3.11 (m, 5H, CH₂), 3.44 (d, 1H, J=13.5 Hz, CH₂), 3.73 (m, 1H, CH₂), 3.84 (m, 4H, CH₂), 4.25 (d, 1H, J=13.5 Hz, CH₂), 6.99 (dd, 2H, J=2.0, 7.0 Hz, Ph-H), 7.24 (d, 1H, J=5.0 Hz, pyrimidinyl-H), 7.44-7.50 (m, 2H, Ph-H), 7.62 (dd, 1H, J=2.0, 6.5 Hz, Ph-H), 8.01 (d, 1H, J=5.5 Hz, Ph-H), 8.16 (s, 1H, Ph-H), 8.38 (d, 1H, J=5.0 Hz, pyrimidinyl-H). MS (ESI⁺) m/z 460.43 [M+H]⁺, C₂₇H₃₃N₅O₂ requires 459.58.

### Example 23

### (1-{3-[2-(6-Methoxy-pyridin-3-ylamino)-pyrimidin-4-yl]-benzyl}-piperidin-2-yl)-methanol (102)

By treatment of 3-dimethylamino-1-[3-(2-hydroxymethyl-piperidin-1-ylmethyl)-phenyl]-propenone with N-(6-methoxy-pyridin-3-yl)-guanidine nitrate. Brown solid. Anal. RP-HPLC: t_{R}=10.1 min (10-70% MeCN, purity 95%). ¹H-NMR (CD₃OD) δ: 1.36 (m, 1H, CH₂), 1.52 (m, 3H, CH₂), 1.78 (m, 2H, CH₂), 2.14 (m, 1H, CH₂), 2.42 (m, 1H, CH₂), 2.84 (m, 1H, CH₂), 3.45 (d, 1H, J=13 Hz, CH₂), 3.73 (dd, 1H, CH₂), 3.84 (m, 1H, CH₂), 3.89 (s, 3H, CH₃), 4.24 (d, 1H J=13.5 Hz, CH₂), 6.82 (d, 1H, J=9.5 Hz, Ph-H), 7.30 (d, 1H, J=5.5 Hz, pyrimidinyl-H), 7.45-7.51 (m, 2H, Ar—H and Ph-H), 8.12 (d, 1H, J=9.5 Hz, Ph-H), 8.06 (d, 1H, J=3.0 Hz, Ph-H), 8.07 (d, 1H, J=3.0 Hz, Ar—H), 8.15 (s, 1H, Ph-H), 8.43 (d, 1H, J=5.0 Hz, Ph-H), 8.53 (d, 1H, J=3.0 Hz, Ar—H). MS (ESI⁺) m/z 406.34 [M+H]⁺, C₂₃H₂₇N₅O₂ requires 405.49.

### Example 24

### 3-{4-[3-(2-Hydroxymethyl-piperidin-1-ylmethyl)-phenyl]-pyrimidin-2-ylamino}-phenol (103)

By treatment of 3-dimethylamino-1-[3-(2-hydroxymethyl-piperidin-1-ylmethyl)-phenyl]-propenone with 3-hydroxyphenyl-guanidine nitrate. Brown solid. Anal. RP-HPLC: t_{R}=9.8 min (10-70% MeCN, purity 100%). ¹H-NMR (CD₃OD) δ: 1.38 (m, 1H, CH₂), 1.45-1.59 (m, 3H, CH₂), 1.72-1.82 (m, 2H, CH₂), 2.15 (m, 1H, CH₂), 2.43 (m, 1H, CH₂), 2.88 (m, 1H, CH₂), 3.45 (d, 1H, J=13 Hz, CH₂), 3.74 (m, 1H, CH₂), 3.85 (m, 1H, CH₂), 4.27 (d, 1H J=13.5 Hz, CH₂), 6.46 (m, 1H, Ph-H), 7.12 (m, 3H, Ph-H), 7.30 (d, 1H, J=5.0 Hz, pyrimidinyl-H), 7.46 (s, 1H, Ph-H), 7.50 (m, 1H, Ph-H), 8.06 (d, 1H, J=7.5 Hz, Ph-H), 8.20 (s, 1H, Ph-H), 8.43 (d, 1H, J=5.5 Hz, pyrimidinyl-H). MS (ESI⁺) m/z 391.42 [M+H]⁺, C₂₃H₂₆N₄O₂ requires 390.48.

### Example 25

### (3-Methanesulfonyl-phenyl)-[4-(3-[1,2,4]triazol-1-ylmethyl-phenyl)-pyrimidin-2-yl]-amine (104)

Brown solid. Anal. RP-HPLC: t_{R}=13.2 min (10-70% MeCN, purity 89%). ¹H-NMR (CDCl₃) δ: 3.07 (s, 3H, CH₃), 7.22 (d, 1H, J=5.5 Hz, pyrimidinyl-H), 7.47-7.51 (m, 3H, Ph-H), 7.93 (m, 2H, Ph-H and Ar—H), 8.32 (s, 1H, Ph-H), 8.33 (s, 1H, Ar—H), 8.45 (d, 1H, J=5.5 Hz, pyrimidinyl-H), 9.10 (s, 1H, Ar—H). MS (ESI⁺) m/z 407.31 [M+H]⁺, C₂₀H₁₈N₆O₂S requires 406.46.

### Example 26

### (1-{3-[2-(3-Nitro-phenylamino)-pyrimidin-4-yl]-benzyl}-piperidin-3-yl)-methanol (105)

Yellow solid. Anal. RP-HPLC: t_{R}=12.9 min (10-70% MeCN, purity>95%). ¹H-NMR (CD₃OD) δ: 0.97 (m, 1H, CH₂), 1.61 (m, 1H, CH₂), 1.68-1.82 (m, 4H, CH₂), 2.05 (m, 1H, CH₂), 2.90 (d, 1H, J=12.5 Hz, CH₂), 3.04 (d, 1H, J=7.5 Hz, CH₂), 3.31 3.42 (m, 1H, CH₂), 3.67 (m, 2H, CH₂), 7.39 (d, 1H, J=5.5 Hz, pyrimidinyl-H), 7.49 (m, 3H, Ph-H), 7.84 (m, 2H, Ph-H), 8.09 (m, 1H, Ph-H), 8.23 (s, 1H, Ph-H), 8.51 (d, 1H, J=4.5 Hz, pyrimidinyl-H), 9.26 (d, 1H, Ph-H). MS (ESI⁺) m/z 420.15 [M+H]⁺, C₂₃H₂₅N₅O₃ requires 419.48.

### Example 27

### 4-{4-[3-(2-Hydroxymethyl-piperidin-1-ylmethyl)-phenyl]-pyrimidin-2-ylamino}-phenol (106)

Brown solid. Anal. RP-HPLC: t_{R}=8.5 min (10-70% MeCN, purity 100%). ¹H-NMR (CD₃OD) δ:1.40 (m, 1H, CH₂), 1.50-1.62 (m, 3H, CH₂), 1.75-1.83 (m, 2H, CH₂), 2.24 (m, 1H, CH₂), 2.53 (m, 1H, CH₂), 2.90 (m, 1H, CH₂), 3.54 (d, 1H, J=13.0 Hz, CH₂), 3.82 (m, 2H, CH₂), 4.30 (d, 1H J=13.5 Hz, CH₂), 6.78 (d, 2H, J=9.0 Hz, Ph-H), 7.23 (d, 1H, J=5.5 Hz, pyrimidinyl-H), 7.46-7.53 (m, 4H, Ph-H), 8.04 (d, 1H, J=9.0 Hz, Ph-H), 8.15 (s, 1H, Ph-H), 8.37 (d, 1H, J=5.5 Hz, pyrimidinyl-H). MS (ESI⁺) m/z 391.25 [M+H]⁺, C₂₃H₂₆N₄O₂ requires 390.48.

### Example 28

### (1-{3-[2-(3,5-Bis-hydroxymethyl-phenylamino)-pyrimidin-4-yl]-benzyl}-piperidin-2-yl)-methanol (107)

Brown solid. Anal. RP-HPLC: t_{R}=8.3 min (10-70% MeCN, purity 90%). ¹H-NMR (CD₃OD) δ: 1.40 (m, 1H, CH₂), 1.50-1.62 (m, 3H, CH₂), 1.75-1.83 (m, 2H, CH₂), 2.24 (m, 1H, CH₂), 2.54 (m, 1H, CH₂), 2.90 (m, 1H, CH₂), 3.57 (d, 1H, J=13.0 Hz, CH₂), 3.80 (m, 2H, CH₂), 4.33 (d, 1H J=13.5 Hz, CH₂), 7.02 (s, 1H, Ph-H), 7.32 (d, 1H, J=5.5 Hz, pyrimidinyl-H), 7.48-7.56 (m, 4H, Ph-H), 7.76 (s, 2H, OH), 8.11 (d, 1H, J=8.0 Hz, Ph-H), 8.23 (s, 1H, Ph-H), 8.46 (d, 1H, J=5.0 Hz, pyrimidinyl-H). MS (ESI⁺) m/z 435.39 [M+H]⁺, C₂₅H₃₀N₄O₃ requires 434.53.

### Example 29

### (1-{3-[2-(4-Methyl-3-nitro-phenylamino)-pyrimidin-4-yl]-benzyl}-piperidin-2-yl)-methanol (108)

Yellow solid. Anal. RP-HPLC: t_{R}=15.2 min (10-70% MeCN, purity 100%). ¹H-NMR (CDCl₃) δ: 1.42 (m, 2H, CH₂), 1.61 (m, 1H, CH₂), 1.74 (m, 3H, CH₂), 2.26 (m, 1H, CH₂), 2.59 (s, 3H, CH3), 2.60 (m, 1H, CH₂), 2.92 (m, 1H, CH₂), 3.53 (d, 1H, J=13.0 Hz, CH₂), 3.60 (dd, 1H, J=4.0, 11.0 Hz, CH₂), 3.91 (dd, 1H, J=4.5, 11.0 Hz, CH₂), 4.24 (d, 1H, J=13.5 Hz, CH₂), 7.26 (d, 1H, J=5.0 Hz, pyrimidinyl-H), 7.28 (d, 1H, J=8.5 Hz, Ph-H), 7.47-7.53 (m, 3H, Ph-H), 7.57 (dd, 1H, J=2.5, 8.5 Hz, Ph-H), 8.65 (s, 1H, Ph-H), 8.97 (d, 1H, J=7.5 Hz, Ph-H), 8.11 (s, 1H, Ph-H), 8.49 (d, 1H, J=5.0 Hz, pyrimidinyl-H), 8.87 (m, 1H, OH). MS (ESI⁺) m/z 434.51 [M+H]⁺, C₂₄H₂₇N₅O₃ requires 433.50.

### Example 30

### 3-[4-(4-Ethoxy-phenyl)-pyrimidin-2-ylamino]-phenol (109)

By treatment of 3-Dimethylamino-1-(4-ethoxy-phenyl)-propenone with 3-hydroxyl-phenyl guanidine nitrate. Brown solid. Anal. RP-HPLC: t_{R}=15.5 min (10-70% MeCN, purity 100%). ¹H-NMR (CDCl₃) δ: 1.44 (t, 3H, J=7.5 Hz, CH₃), 4.08 (q, 2H, J=7.0 Hz, CH₂), 6.54 (dd, 1H, J=2.0, 7.0 Hz, Ph-H), 6.98 (m, 2H, Ph-H), 7.07 (d, 1H, J=5.5 Hz, pyrimidinyl-H), 7.10 (s, 1H, OH), 7.18 (t, 1H, J=8.5 Hz, Ph-H), 7.32 (s, 1H, Ph-H), 7.42 (m, 1H, Ph-H), 8.01 (d, 2H, J=8.5 Hz, Ph-H), 8.38 (1H, d, J=5.0 Hz, pyrimidinyl-H). MS (ESI⁺) m/z 308.40 [M+H]⁺, C₁₈H₁₇N₃O₂ requires 307.35.

### Example 31

### 4-[4-(4-Methoxy-phenyl)-pyrimidin-2-ylamino]-phenol (110)

Yellow solid. Anal. RP-HPLC: t_{R}=12.9 (10-70% MeCN, purity 100%). ¹H-NMR (CDCl₃) δ: 3.82 (s, 3H, CH₃), 6.79 (m, 2H, Ph-H), 6.95 (m, 2H, Ph-H), 6.99 (m, 1H, pyrimidinyl-H), 7.40 (m, 2H, Ph-H), 7.96 (m, 2H, Ph-H), 8.25 (m, 1H, pyrimidinyl-H). MS (ESI⁺) m/z 294.15 [M+H]⁺, C₁₇H₁₅N₃O₂ requires 293.32.

### Example 32

### [4-(4-Methoxy-phenyl)-pyrimidin-2-yl]-(4-morpholin-4-yl-phenyl)-amine (111)

Yellow solid. Anal. RP-HPLC: t_{R}=13.8 min (10-70% MeCN, purity 100%). ¹H-NMR (DMSO-d₆) δ: 3.04 (m, 4H, CH₂), 3.74 (m, 4H, CH₂), 3.83 (s, 3H, CH₃), 6.92 (d, 2H, J=9.0 Hz, Ph-H), 7.08 (d, 2H, J=8.5 Hz, Ph-H), 7.25 (d, 1H, J=5.0 Hz, pyrimidinyl-H), 7.66 (d, 2H, J=9.5 Hz, Ph-H), 8.12 (d, 1H, J=9.0 Hz, Ph-H), 8.41 (d, 1H, J=5.5 Hz, pyrimidinyl-H), 9.33 (s, 1H, NH). MS (ESI⁺) m/z 363.09 [M+H]⁺, C₂₁H₂₂N₄O₂ requires 362.43.

### Example 33

### [4-(4-Methoxy-phenyl)-pyrimidin-2-yl]-(6-methoxy-pyridin-3-yl)-amine (124)

Yellow solid. Anal. RP-HPLC: t_{R}=15.2 min (10-70% MeCN, purity 100%). 1H-NMR (DMSO-d₆) δ: 3.83 (s, 3H, CH₃), 3.84 (s, 3H, CH₃), 6.81 (d, 1H, J=9.0 Hz, Ar—H), 7.09 (d, 2H, J=9.0 Hz, Ph-H), 7.32 (d, 1H, J=5.0 Hz, pyrimidinyl-H), 8.06 (dd, 1H, J=2.5, 9.0 Hz, Ar—H), 8.11 (dd, 2H, J=2.5, 9.0 Hz, Ph-H), 8.44 (d, 1H, J=5.5 Hz, pyrimidinyl-H), 8.56 (d, 1H, J=2.5 Hz, Ar—H), 9.50 (s, 1H, NH). MS (ESI⁺) m/z 406.34 [M+H]⁺, C₁₇H₁₆N₄O₂ requires 308.33.

### Example 34

### (3-[2-(6-Methoxy-pyridin-3-ylamino)-pyrimidin-4-yl]-phenyl-methanol (125)

Yellow solid. Anal. RP-HPLC: t_{R}=11.3 min (10-70% MeCN, purity 100%). ¹H-NMR (DMSO-d₆) δ: 3.83 (s, 3H, CH₃), 4.59 (d, 2H, J=6.5 Hz, CH₂), 6.81 (d, 1H, J=9.5 Hz, Ar—H), 7.36 (d, 1H, J=5.5 Hz, pyrimidinyl-H), 7.49 (m, 2H, Ph-H), 7.99 (m, 1H, Ar—H), 8.10 (m, 2H, Ph-H and Ar—H), 8.51 (d, 1H, J=5.0 Hz, pyrimidinyl-H), 8.55 (d, 1H, J=2.5 Hz, Ar—H), 9.59 (s, 1H, NH). MS (ESI⁺) m/z 309.43 [M+H]⁺, C₁₇H₁₆N₄O₂ requires 308.33.

### Example 35

### (3-Nitro-phenyl)-{4-[4-(2-[1,2, 4]triazol-1-yl-ethyl)-phenyl]-pyrimidin-2-yl}-amine (126)

Yellow solid. Anal. RP-HPLC: t_{R}=17.8 min (10-70% MeCN, purity 100%). ¹H-NMR (CDCl₃) δ: 3.25 (t, 2H, J=7.0 Hz, CH₂), 4.44 (t, 2H, J=7.0 Hz, CH₂), 7.21 (d, 2H, J=8.5 Hz, Ph-H), 7.22 (d, 1H, J=5.5 Hz, pyrimidinyl-H), 7.46 (t, 1H, J=8.0 Hz, Ph-H), 7.74 (d, 1H, J=8.5 Hz, Ph-H), 7.80 (s, 1H, Ar—H), 7.87 (d, 1H, J=8.5 Hz, Ph-H), 7.95 (m, 2H, Ar—H and Ph-H), 8.04 (d, 2H, J=8.0 Hz, Ph-H), 8.47 (d, 1H, J=5.5Hz, pyrimidinyl-H), 9.14 (sbr, 1H, NH). MS (ESI⁺) m/z 388.48 [M+H]⁺, C₂₀H₁₇N₇O₂ requires 387.39.

### Example 36

### (1-{4-[2-(3-Nitro-phenylamino)-pyrimidin-4-yl]-benzyl}-piperidin-2-yl)-methanol (127)

Yellow solid. Anal. RP-HPLC: t_{R}=13.3 min (10-70% MeCN, purity 96%). ¹H-NMR (CDCl₃) δ:1.39 (m, 2H, CH₂), 1.57 (m, 1H, CH₂), 1.70 (m, 3H, CH₂), 2.18 (m, 1H, CH₂), 2.50 (m, 1H, CH₂), 2.89(m, 1H, CH₂), 3.41 (d, 1H, J=13.5 Hz, CH₂), 3.57 (dd, 1H, J=4.0, 11.0 Hz, CH₂), 3.88 (dd, 1H, J=4.5, 11.0 Hz, CH₂), 4.15 (d, 1H, J=13.0 Hz, CH₂), 7.26 (d, 1H, J=5.5 Hz, pyrimidinyl-H), 7.48 (m, 3H, Ph-H), 7.78 (dd, 2H, J=2.5, 7.5 Hz, Ph-H), 7.82 (s, 1H, Ph-H), 7.87 (dd, 1H, J=2.5, 7.5 Hz, Ph-H), 8.09 (d, 2H, J=7.5 Hz, Ph-H), 8.52 (d, 1H, J=5.5 Hz, pyrimidinyl-H), 9.05 (m, 1H, OH/NH). ¹³C-NMR (DMSO-d₆) δ: 23.76, 25.72, 29.22, 52.48, 58.41, 63.27, 63.75, 109.44, 113.05, 116.17, 125.26, 127.48, 129.62, 130.37, 135.32, 142.68, 144.47, 148.83, 159.81, 160.41, 164.32. MS (ESI⁺) m/z 420.40 [M+H]⁺, C₂₃H₂₅N₅O₃ requires 419.48.

### Example 37

### [4-(4-Methoxy-phenyl)-pyrimidin-2-yl]-(3,4,5-trimethoxy-phenyl)-amine (128)

Yellow solid. Anal. RP-HPLC: t_{R}=15.2 min (10-70% MeCN, purity 94%). ¹H-NMR (DMSO-d₆) δ: 3.62 (s, 3H, CH₃), 3.79 (s, 6H, CH₃), 3.84 (s, 3H, CH₃), 7.09 (d, 2H, J=9.0 Hz, Ph-H), 7.30 (s, 2H, Ph-H), 7.34 (d, 1H, J=5.5 Hz, pyrimidinyl-H), 8.16 (d, 2H, J=9.5 Hz, Ph-H), 8.47 (d, 1H, J=5.0 Hz, pyrimidinyl-H), 9.46 (s, 1H, NH). MS (ESI⁺) m/z 366.47 [M+H]⁺, C₂₀H₂₁N₃O₄ requires 367.40.

### Example 38

### N-Methyl-N-{3-[2-(3-nitro-phenylamino)-pyrimidin-4-yl]-phenyl}-methanesulfonamide (129)

Yellow solid. Anal. RP-HPLC: t_{R}=17.0 min (10-70% MeCN, purity 100%). ¹H-NMR (DMSO-d₆) δ: 3.01 (s, 3H, CH₃), 3.33 (s, 3H, CH₃), 7.58-7.62 (m, 4H, Ph-H and pyrimidinyl-H), 7.83 (dd, 1H, J=2.5, 8.5 Hz, Ph-H), 8.15 (m, 2H, Ph-H), 8.20 (s, 1H, Ph-H), 8.68 (d, 1H, J=5.5 Hz, pyrimidinyl-H), 9.03 (m, 1H, Ph-H), 10.29 (s, 1H, NH). MS (ESI⁺) m/z 400.50 [M+H]⁺, C₁₈H₁₇N₅O₄S requires 399.42.

### Example 39

### N-{3-[2-(3-Hydroxy-phenylamino)-pyrimidin-4-yl]-phenyl}-N-methyl-methanesulfonamide (130)

Yellow solid. Anal. RP-HPLC: t_{R}=12.9 min (10-70% MeCN, purity 92%). ¹H-NMR (DMSO-d₆) δ: 3.01 (s, 3H, CH₃), 3.32 (s, 3H, CH₃), 7.38 (m, 1H, Ph-H), 7.06 (t, 1H, J=8.5 Hz, Ph-H), 7.25 (m, 1H, Ph-H), 7.37 (m, 1H, Ph-H), 7.42 (d, 1H, J=5.0 Hz, pyrimidinyl-H), 7.59 (m, 1H, Ph-H), 8.09 (m, 1H, Ph-H), 8.19 (s, 1H, Ph-H), 8.56 (d, 1H, J=5.5 Hz, pyrimidinyl-H), 9.25 (s 1H, Ph-H), 9.59 (s, H, NH). MS (ESI⁺) m/z 371.41 [M+H]⁺, C₁₈H₁₈N₄O₃S requires 370.43.

### Example 40

### N-{3-[2-(4-Hydroxy-phenylamino)-pyrimidin-4-yl]-phenyl}-N-methyl-methanesulfonamide (131)

Yellow solid. Anal. RP-HPLC: t_{R}=11.0 min (10-70% MeCN, purity 93%). ¹H-NMR (CDCl₃) δ: 2.86 (s, 3H, CH₃), 3.37 (s, 3H, CH₃), 6.82 (m, 2H, Ph-H), 7.08 (d, 1H, J=5.0 Hz, pyrimidinyl-H), 7.44 (m, 2H, Ph-H), 7.49 (m, 2H, Ph-H), 7.88 (m, 1H, Ph-H), 8.13 (s, 1H, Ph-H), 8.38 (d, 1H, J=5.0 Hz, pyrimidinyl-H). MS (ESI⁺) m/z 371.41 [M+H]⁺, C₁₈H₁₈N₄O₃S requires 370.43.

### Example 41

### N-{3-[2-(6-Methoxy-pyridin-3-ylamino)-pyrimidin-4-yl]-phenyl}-N-methyl-methanesulfonamide (132)

Yellow solid. Anal. RP-HPLC: t_{R}=12.9 min (10-70% MeCN, purity 94%). ¹H-NMR (CDCl₃) δ: 2.88 (s, 3H, CH₃), 3.38 (s, 3H, CH₃), 3.93 (s, 3H, CH₃), 6.77 (d, 1H, J=9.0 Hz, Ph-H), 7.14 (d, 1H, J=5.5 Hz, pyrimidinyl-H), 7.50 (m, 2H, Ph-H), 7.92 (m, 1H, Ph-H), 7.99 (dd, 1H, J=2.0, 8.5 Hz, Ph-H), 8.11 (s, 1H, Ph-H), 8.37 (d, 1H, J=2.5 Hz, Ph-H), 8.44 (d, 1H, J=5.5 Hz, pyrimidinyl-H). MS (ESI⁺) m/z 386.40 [M+H]⁺, C₁₈H₁₉N₅O₃S requires 385.44.

### Example 42

### General Conditions for the Following Examples (43-45)

Microwave reactions were performed using a CEM Discover or Explorer System. HPLC separation was achieved using a Biotage ParallexFLEX system with an automated (UV detection) fraction collector using a SUPLELCOSIL C18 reversed phase preparative column, and gradient elution with water (containing 0.05% CF₃COOH)—acetonitrile as solvents. HPLC samples were evaporated in vacuo using a CHRIST Beta-RVC centrifuge-evaporator system. Electrospray mass spectrometry was performed using a Micromass Platform II machine. NMR spectra were recorded using a Brucker ARX 250 (MHz) instruments.

### Example 43

### 3-[4-(2,5-Dimethyl-phenyl)-pyrimidin-2-ylamino]-phenol (62)

A mixture of 2,4-dichloropyrimidine (50 mg, 0.33 mmol), 2,5-dimethylphenylboronic acid (50 mg, 0.33 mmol), caesium carbonate (136 mg, 1.0 mmol), palladium (II) acetate (5 mg, 0.02 mmol), acetonitrile (2 mL) and water (0.2 mL) in a 10-mL microwave tube was sealed and heated in the microwave at 130° C. for 15 min. Upon cooling the organic phase was transferred into another microwave tube, to which was added 3-aminophenol (55 mg, 0.50 mmol) and toluene-4-sulfonic acid monohydrate (95 mg, 0.50 mmol). The tube was resealed and irradiated at 130° C. in the microwave for 15 min. The reaction mixture was filtered and purified by HPLC to give 58 mg (61%) of the title compound. ¹H-NMR (MeCN-d₃) δ: 2.55 (s, 3H, CH₃), 2.58 (s, 3H, CH₃), 6.78-8.59 (m, 9H, Ar—H), 10.97 (s, 1H, NH). MS (ESI⁺) m/z 292 [M+H]⁺, C₁₈H₁₇N₃O requires 291.35).

### Example 44

### 3-[2-(3-Nitro-phenylamino)-pyrimidin-4-yl]-phenol (55)

To a microwave tube was added 2,4-dichloropyrimidine (0.075 g, 0.50 mmol), 3-hydroxyphenylboronic acid (0.069 g, 0.50 mmol), palladium (II) acetate (0.011 g, 0.05 mmol), caesium carbonate (0.245 g, 0.75 mmol), MeCN (3 mL) and H₂O (0.5 mL). The vessel was sealed and irradiated in the microwave at 130° C. for 15 min. On cooling, the reaction mixture (approx. 0.17 mmol) was transferred to another microwave tube. To this a mixture of 3-nitroaniline (0.028 g, 0.2 mmol) and toluene-4-sulfonic acid monohydrate (0.065 g, 0.34 mmol) and MeCN (1 mL) was added. The vessel was sealed and irradiated in the microwave at 130° C. for 15 min. On cooling the reaction mixture was filtered and purified by HPLC to afford 20 mg of the title compound. Yield 38%; 1H-NMR (CD₃OD) δ: 6.92-9.08 (m, 10H, Ar—H). MS (ESI⁺) m/z 309 [M+H]⁺, C₁₆H₁₂N₄O₃ requires 308.29.

### Example 45

The following compounds were prepared in a similar manner as described in Examples 43 and 44:

### [3-[2-(3-Hydroxy-phenylamino)-pyrimidin-4-yl]-phenol (56)

Yield 65%; ¹H-NMR (CD₃OD) δ: 6.92-8.71 (m, 10H, Ar—H). MS (ESI⁺) m/z 280 [M+H]⁺, C₁₆H₁₃N₃O₂ requires 279.29.

### 3-[2-(3-Fluoro-phenylamino)-pyrimidin-4-yl]-phenol (57)

Yield 61%; ¹H-NMR (CD₃OD) δ: 6.93-8.55 (m, 10H, Ar—H), 10.34 (s, 1H, OH). MS (ESI⁺) m/z 282 [M+H]⁺, C₁₆H₁₂FN₃O requires 281.28.

### 3-[4-(3-Nitro-phenyl)-pyrimidin-2-ylamino]-phenol (64)

Yield 53%; ¹H-NMR (CD₃OD) δ: 6.35-8.87 (m, 10H, Ar—H). MS (ESI⁺) m/z 309 [M+H]⁺, C₁₆H₁₂N₄O₃ requires 308.29.

### N-{3-[2-(3-Hydroxy-phenylamino)-pyrimidin-4-yl]-phenyl}-acetamide (67)

Yield 12%; ¹H-NMR (CD₃OD) δ: 2.28 (s, 3H, CH₃), 6.33-9.54 (m, 10H, Ar—H). MS (ESI⁺) m/z 321 [M+H]⁺, C18H₁₆N₄O₂ requires 320.35.

### N-{3-[2-(3-Nitro-phenylamino)-pyrimidin-4-yl]-phenyl}-acetamide (69)

Yield 24%; ¹H-NMR (CD₃OD) δ: 2.21 (s, 3H, CH₃), 7.47-9.14 (m, 10H, Ar—H). MS (ESI⁺) m/z 350 [M+H]⁺, C₁₈H₁₅N₅O₃ requires 349.34.

### 3-[2-(3-Hydroxymethyl-phenylamino)-pyrimidin-4-yl]-phenol (72)

Yield 14%; ¹H-NMR (CD₃OD) δ: 2.39 (s, 1H, OH), 4.70 (s, 2H, CH₂), 8.40-7.02 (m, 10H, Ar—H). MS (ESI⁺) m/z 294 [M+H]⁺, C₁₇H₁₅N₃O₂ requires 293.32.

### 3-[4-(3-Methoxy-phenyl)-pyrimidin-2-ylamino]-phenol (88)

Yield 23%; ¹H-NMR (CD₃OD) δ: 3.93 (s, 3H, CH₃), 6.64-8.41 (m, 10H, Ar—H). MS (ESI⁺) m/z 294 [M+H]⁺, C₁₇H₁₅N₃O₂ requires 293.32.

### {3-[2-(3-Nitro-phenylamino)-pyrimidin-4-yl]-phenyl}-methanol (91)

Yield 15%; ¹H-NMR (CD₃OD) δ: 4.65 (s, 2H, CH₂), 7.34-9.14 (m, 10H, Ar—H). MS (ESI⁺) m/z 323 [M+H]⁺, C₁₇H₁₄N₄O₃ requires 322.32.

### [4-(4-Methoxy-phenyl)-pyrimidin-2-yl]-(3-nitro-phenyl)-amine (94)

Yield 43%; ¹H-NMR (CD₃OD) δ: 3.82 (s, 3H, CH₃), 6.96-9.14 (m, 10H, Ar—H). MS (ESI⁺) m/z 323 [M+H]⁺, C₁₇H₁₄N₄O₃ requires 322.32.

### 3-[4-(3-trifluoromethyl-phenyl)-pyrimidin-2-ylamino]-phenol (95)

Yield 38%; ¹H-NMR (CD₃OD) δ: 6.76-8.71 (m, 10H, Ar—H). MS (ESI⁺) m/z 332 [M+H]⁺, C₁₇H₁₂F₃N₃O requires 331.29.

### 4-[4-(3-trifluoromethyl-phenyl)-pyrimidin-2-ylamino]-phenol (96)

Yield 49%; ¹H-NMR (CD₃OD) δ: 6.75-8.43 (m, 10H, Ar—H). MS (ESI⁺) m/z 332 [M+H]⁺, C₁₇H₁₂F₃N₃O requires 331.29.

### 4-[4-(3-Methoxy-phenyl)-pyrimidin-2-ylamino]-phenol (98)

Yield 25%; ¹H-NMR (CD₃OD) δ: 3.92 (s, 3H, CH₃), 6.89-8.38 (m, 10H, Ar—H). MS (ESI⁺) m/z 294 [M+H]⁺, C₁₇H₁₅N₃O₂ requires 293.32.

### [4-(3-Chloro-phenyl)-pyrimidin-2-yl]-(3-nitro-phenyl)-amine (112)

Yield 19%; ¹H-NMR (CD₃OD) δ: 7.33-9.08 (m, 10H, Ar—H). MS (ESI⁺) m/z 326 [M+H]⁺, C₁₆H₁₁ClN₄O₂ requires 326.74.

### 3-[4-(2,5-Difluoro-phenyl)-pyrimidin-2-ylamino]-phenol (114)

Yield 17%; ¹H-NMR (CD₃OD) δ: 6.63-8.59 (m, 10H, Ar—H). MS (ESI⁺) m/z 300 [M+H]⁺, C₁₆H₁₁F₂N₃O requires 299.27.

### {3-[2-(3-Fluoro-phenylamino)-pyrimidin-4-yl]-phenyl}-methanol (116)

Yield 22%; MS (ESI⁺) m/z 295 [M+H]⁺, C₁₇H₁₄FN₃O requires 295.31.

### (3-Fluoro-phenyl)-[4-(3-methoxy-phenyl)-pyrimidin-2-yl]-amine (118)

Yield 34%; ¹H-NMR (CDCl₃) δ: 3.84 (s, 3H, CH₃), 6.66-8.42 (m, 10H, Ar—H). MS (ESI⁺) m/z 296 [M+H]⁺, C₁₇H₁₄FN₃O requires 295.31.

### (3-Fluoro-phenyl)-[4-(4-methoxy-phenyl)-pyrimidin-2-yl]-amine (119)

Yield 34%; ¹H-NMR (CDCl₃) δ: 3.95 (s, 3H, CH₃), 6.83-8.40 (m, 10H, Ar—H). MS (ESI⁺) m/z 296 [M+H]⁺, C₁₇H₁₄FN₃O requires 295.31.

### 3-[2-(4-Hydroxy-phenylamino)-pyrimidin-4-yl]-phenol (122)

Yield 65%; ¹H-NMR (CD₃OD) δ: 6.92-8.71 (m, 10H, Ar—H). MS MS (ESI⁺) m/z 280 [M+H]⁺, C₁₆H₁₃N₃O₂ requires 279.29.

### Example 46

Kinase Assays

The compounds from the examples above were investigated for their ability to inhibit the enzymatic activity of various protein kinases. This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 MM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C. Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%.). IC₅₀ values for selected compounds of the invention are shown in Table 1.

MTT Cytotoxicity Assay

The compounds from the examples above were subjected to a standard cellular proliferation assay using human tumour cell lines obtained from the ATCC (American Type Culture Collection, 10801 University Boulevard, Manessas, Va. 20110-2209, USA). Standard 72-h MTT (thiazolyl blue; 3-[4,5-dimethylthiazol-2-yl]-2,5-diphenyltetrazolium bromide) assays were performed [67, 68]. In short: cells were seeded into 96-well plates according to doubling time and incubated overnight at 37° C. Test compounds were made up in DMSO and a 1/3 dilution series prepared in 100 μL cell media, added to cells (in triplicates) and incubated for 72 ho at 37° C. MTT was made up as a stock of 5 mg/mL in cell media and filter-sterilised. Media was removed from cells followed by a wash with 200 μL PBS. MTT solution was then added at 20 μL per well and incubated in the dark at 37° C. for 4 h. MTT solution was removed and cells again washed with 200 μL PBS. MTT dye was solubilised with 200 μL per well of DMSO with agitation. Absorbance was read at 540 nm and data analysed using curve-fitting software (GraphPad Prism version 3.00 for Windows, GraphPad Software, San Diego Calif. USA) to determine IC₅₀ values (concentration of test compound which inhibits cell growth by 50%). IC₅₀ values for selected compounds of the invention are shown in Table 2.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Structures of exemplified compounds and inhibitory activity against various protein kinases.

| | | | Kinase inhibition IC₅₀ (μM) | | | | | | | | |
|---|---|---|---|---|---|---|---|---|---|---|---|
| | | | CDK1/ | CDK2/ | CDK2/ | CDK4/ | CDK7/ | CDK9/ | | | |
| No. | Structure | Name | cyclin B | cyclin A | cyclin E | cyclin D1 | cyclin H | cyclin T1 | GSK3β | PLK-1 | Aurora-A |
| 1 | 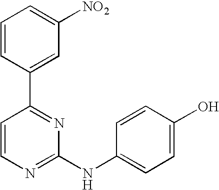 | 4-[4-(3-Nitro-phenyl)- pyrimidin-2-ylamino]- phenol | | | 1.6 | | | | | | |
| 2 | 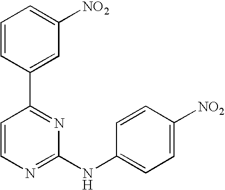 | (4-Nitro-phenyl)-[4-(3- nitro-phenyl)-pyrimidin-2-yl]-amine | | | | | | | | 6.5 | |
| 3 | 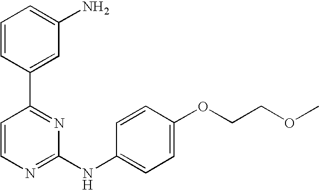 | [4-(3-Amino-phenyl)- pyrimidin-2-yl]-[4-(2- methoxy-ethoxy)-phenyl]-amine | | | | | | 0.018 | | | |
| 4 | 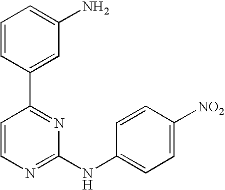 | [4-(3-Amino-phenyl)- pyrimidin-2-yl]-(4- nitro-phenyl)-amine | | | | | | | | | |
| 5 | 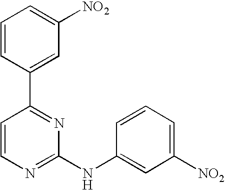 | (3-Nitro-phenyl)-[4-(3- nitro-phenyl)-pyrimidin-2-yl]-amine | | | | | | | | | |
| 6 | 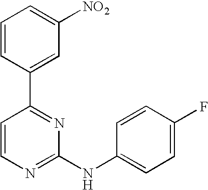 | (4-Fluoro-phenyl)-[4- (3-nitro-phenyl)-pyrimidin-2-yl]-amine | | | | | | | | | |
| 7 | 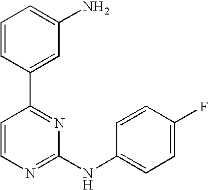 | [4-(3-Amino-phenyl)- pyrimidin-2-yl]-(4-fluoro-phenyl)-amine | | | | | | | | | |
| 8 | 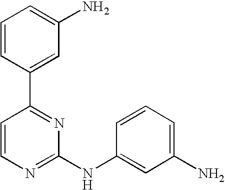 | N-[4-(3-Amino- phenyl)-pyrimidin-2- yl]-benzene-1,3-diamine | | | | | | | | | |
| 9 | 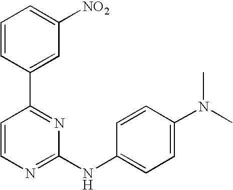 | N,N-Dimethyl-N′-[4-(3-nitro-phenyl)- pyrimidin-2-yl]-benzene-1,4-diamine | | | | | | | | | |
| 10 | 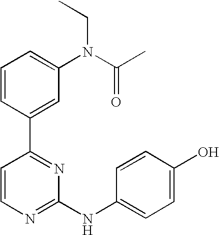 | N-Ethyl-N-{3-[2-(4-hydroxy- phenylamino)-pyrimidin- 4-yl]-phenyl)-acetamide | | | 0.16 | | | | | 4.8 | |
| 11 | 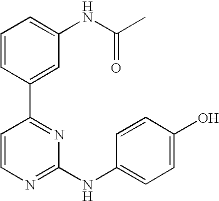 | N-{3-[2-(4-Hydroxy- phenylamino)-pyrimidin- 4-yl]-phenyl}-acetamide | | | 0.25 | | | | | 6.9 | |
| 12 | 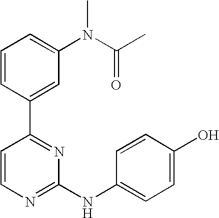 | N-{3-[2-(4-Hydroxy- phenylamino)-pyrimidin-4-yl]- phenyl}-N-methyl-acetamide | | | 0.24 | | | | | | |
| 13 | 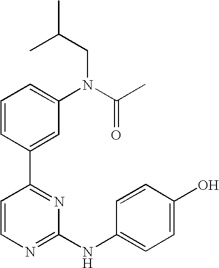 | N-{3-[2-(4-Hydroxy- phenylamino)-pyrimidin-4-yl]- phenyl}-N-isobutyl-acetamide | | | 0.55 | | | | | 14.5 | |
| 14 | 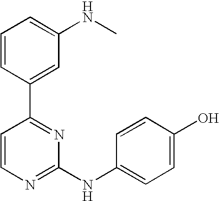 | 4-[4-(3-Methylamino- phenyl)-pyrimidin-2-ylamino]-phenol | | | 0.045 | | | | | 13.7 | |
| 15 | 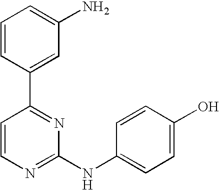 | 4-[4-(3-Amino- phenyl)-pyrimidin-2-ylamino]-phenol | | | 0.36 | | | | | 7.8 | |
| 16 | 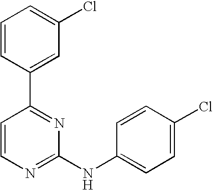 | (4-Chloro-phenyl)-[4- (3-chloro-phenyl)- pyrimidin-2-yl]-amine | | | | | | | | 8.7 | |
| 17 | 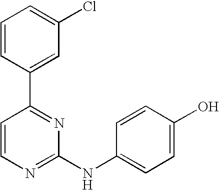 | 4-[4-(3-Chloro- phenyl)-pyrimidin-2-ylamino]-phenol | 5.3 | 2.1 | 0.39 | | | | | | |
| 18 | 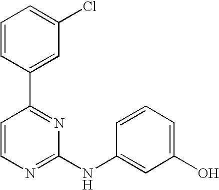 | 3-[4-(3-Chloro- phenyl)-pyrimidin-2-ylamino]-phenol | 5.5 | 3.1 | 1.1 | | | | | | |
| 19 | 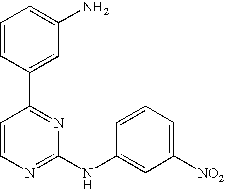 | [4-(3-Amino-phenyl)- pyrimidin-2-yl]-(3-nitro-phenyl)-amine | | | | | 2.5 | | 0.055 | | |
| 20 | 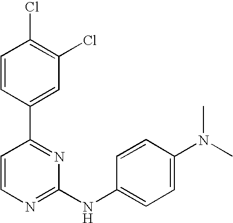 | N-[4-(3,4-Dichloro-phenyl)- pyrimidin-2-yl]-N′,N′-dimethyl- benzene-1,4-diamine | 3.9 | | 6.1 | 20 | | | 1.1 | | |
| 21 | 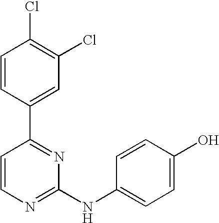 | 4-[4-(3,4-Dichloro- phenyl)-pyrimidin-2-ylamino]-phenol | 29 | | 3.5 | 29 | | 5.4 | | | |
| 22 | 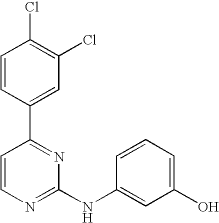 | 3-[4-(3,4-Dichloro- phenyl)-pyrimidin-2-ylamino]-phenol | | | | | | | | | |
| 23 | 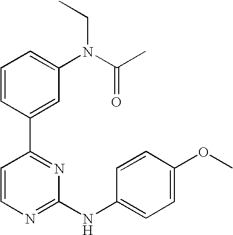 | N-Ethyl-N-{3-[2-(4- methoxy-phenylamino)-pyrimidin- 4-yl]-phenyl}-acetamide | 3.6 | 1.2 | 1.1 | 7.6 | 4.3 | 0.40 | 0.12 | | |
| 24 | 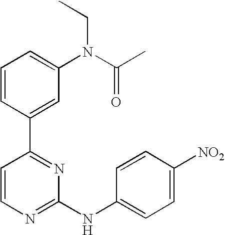 | N-Ethyl-N-{3-[2-(4-nitro- phenylamino)-pyrimidin-4- yl]-phenyl}-acetamide | | | | | | | | | |
| 25 | 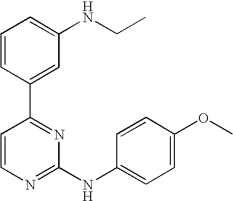 | [4-(3-Ethylamino-phenyl)- pyrimidin-2-yl]-(4-methoxy- phenyl)-amine | 0.17 | 0.03 | 0.039 | 0.17 | 0.94 | 0.005 | 0.14 | | |
| 26 | 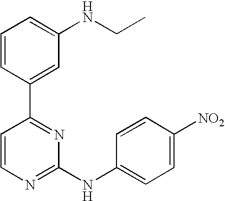 | [4-(3-Ethylamino-phenyl)- pyrimidin-2-yl]-(4-nitro-phenyl)-amine | 1.3 | 0.13 | 0.033 | | 20 | 0.059 | 0.040 | | |
| 27 | 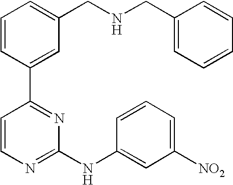 | {4-[3-(Benzylamino-methyl)- phenyl]-pyrimidin-2-yl}- (3-nitro-phenyl)-amine | | 6.5 | 0.57 | 16 | 6.0 | 2.0 | 0.18 | | |
| 28 | 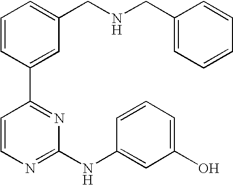 | 3-{4-[3-(Benzylamino- methyl)-phenyl]-pyrimidin- 2-ylamino}-phenol | 2.1 | 1.0 | 0.86 | 2.6 | 2.6 | 0.035 | 0.12 | | |
| 29 | 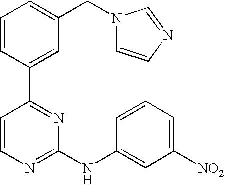 | [4-(3-Imidazol-1-ylmethyl- phenyl)-pyrimidin-2-yl]-(3- nitro-phenyl)-amine | 2.2 | 5.1 | 0.14 | 62 | 0.071 | 0.044 | 0.050 | | |
| 30 | 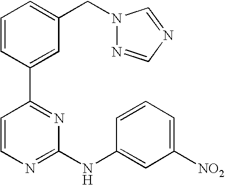 | (3-Nitro-phenyl)-[4-(3- [1,2,4]triazol-1-ylmethyl- phenyl)-pyrimidin-2-yl]-amine | | | 0.040 | | 0.57 | 0.002 | 0.019 | | |
| 31 | 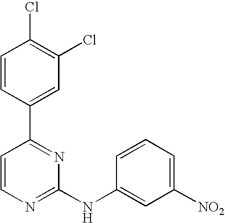 | [4-(3,4-Dichloro-phenyl)- pyrimidin-2-yl]-(3- nitro-phenyl)-amine | | | | | | | 4.5 | | |
| 32 | 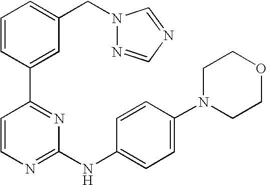 | (4-Morpholin-4-yl-phenyl)- [4-(3-[1,2,4]triazol-1- ylmethyl-phenyl)-pyrimidin-2-yl]-amine | | | 4.4 | 2.2 | 4.0 | 0.42 | 0.37 | | |
| 33 | 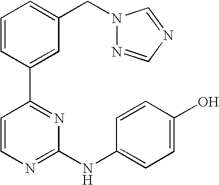 | 4-[4-(3-[1,2,4]Triazol- 1-ylmethyl-phenyl)-pyrimidin- 2-ylamino]-phenol | | 2.0 | 0.14 | 9.1 | 1.1 | 0.092 | 0.12 | | |
| 34 | 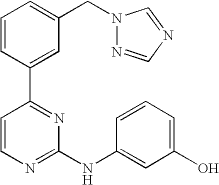 | 3-[4-(3-[1,2,4]Triazol- 1-ylmethyl-phenyl)-pyrimidin- 2-ylamino]-phenol | 7.0 | 2.4 | 0.24 | 1.8 | 1.2 | 0.13 | 0.041 | | |
| 35 | 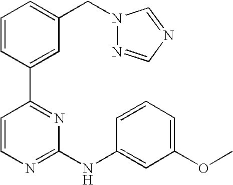 | (3-Methoxy-phenyl)-[4-(3- [1,2,4]triazol-1-ylmethyl- phenyl)-pyrimidin-2-yl]-amine | | 3.2 | 0.92 | 0.57 | 0.58 | 0.069 | 0.10 | | |
| 36 | 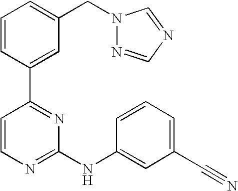 | 3-[4-(3-[1,2,4]Triazol-1- ylmethyl-phenyl)-pyrimidin-2- ylamino]-benzonitrile | | 3.9 | 0.079 | 4.7 | 0.32 | 0.009 | 0.032 | | |
| 37 | 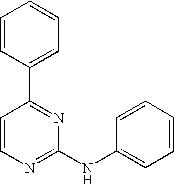 | Phenyl-(4-phenyl- pyrimidin-2-yl)-amine | | | 18 | | | | | | |
| 38 | 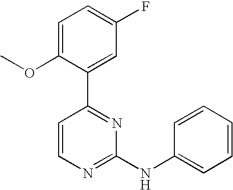 | [4-(5-Fluoro-2-methoxy- phenyl)-pyrimidin-2- yl]-phenyl-amine | | | | | | | | | |
| 39 | 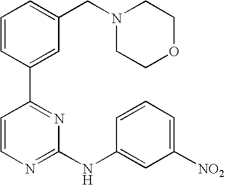 | [4-(3-Morpholin-4-yl- methyl-phenyl)-pyrimidin- 2-yl]-(3-nitro-phenyl)-amine | 0.35 | 2.4 | 0.12 | 2.41 | 0.038 | 0.11 | 0.15 | | |
| 40 | 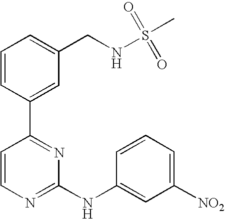 | N-{3-[2-(3-Nitro-phenyl- amino)-pyrimidin-4-yl]- benzyl}-methanesulfonamide | 0.27 | | 0.42 | | 0.88 | 1.6 | 0.005 | | |
| 41 | 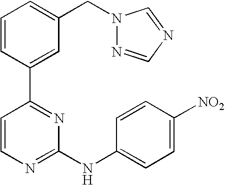 | (4-Nitro-phenyl)-[4-(3- [1,2,4]triazol-1-ylmethyl- phenyl)-pyrimidin-2-yl]-amine | | | 0.42 | | 5.5 | 0.92 | 0.043 | | |
| 42 | 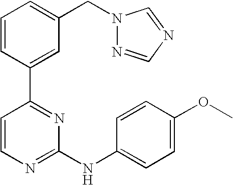 | (4-Methoxy-phenyl)-[4-(3- [1,2,4]triazol-1-ylmethyl- phenyl)-pyrimidin-2-yl]-amine | 11 | | 3.2 | 2.0 | 1.1 | 0.22 | 0.087 | | |
| 43 | 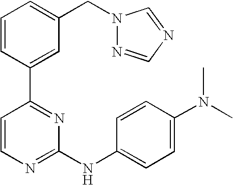 | N,N-Dimethyl-N′-[4-(3- [1,2,4]triazol-1-ylmethyl-phenyl)- pyrimidin-2-yl]-benzene-1,4-diamine | | | 3.7 | 1.9 | 1.4 | 0.53 | 0.19 | | |
| 44 | 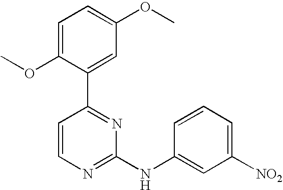 | [4-(2,5-Dimethoxy-phenyl)- pyrimidin-2-yl]-(3-nitro- phenyl)-amine | | | 17 | | | | 0.49 | | |
| 45 | 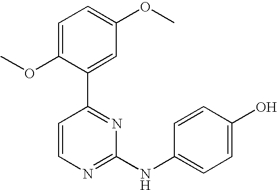 | 4-[4-(2,5-Dimethoxy- phenyl)-pyrimidin-2- ylamino]-phenol | | | 5.8 | 28 | 16 | 25 | 3.6 | | |
| 46 | 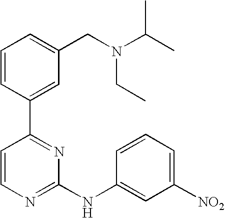 | (4-{3-[(Ethyl-isopropyl- amino)-methyl]-phenyl}- pyrimidin-2-yl)-(3-nitro-phenyl)-amine | | 0.48 | 0.17 | 1.4 | 0.13 | 0.002 | 0.10 | | 0.03 |
| 47 | 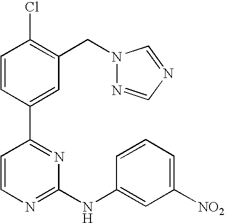 | [4-(4-Chloro-3-[1,2,4]- triazol-1-ylmethyl-phenyl)- pyrimidin-2-yl]-(3-nitro-phenyl)-amine | | | 1.4 | 28 | 20 | 1.3 | 0.13 | | |
| 48 | 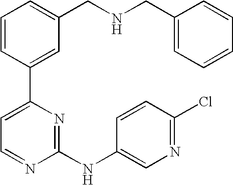 | {4-[3-(Benzylamino-methyl)- phenyl]-pyrimidin-2-yl}- (6-chloro-pyridin-3-yl)-amine | | 9.8 | 1.3 | 3.5 | 1.7 | 1.2 | 0.32 | | |
| 49 | 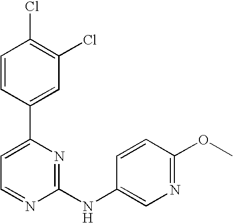 | [4-(3,4-Dichloro-phenyl)- pyrimidin-2-yl]-(6-methoxy- pyridin-3-yl)-amine | | | | | | | 5.2 | | |
| 50 | 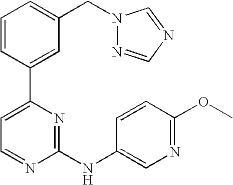 | (6-Methoxy-pyridin-3- yl)-[4-(3-[1,2,4]triazol- 1-ylmethyl-phenyl)-pyrimidin- 2-yl]-amine | | | 1.8 | 9.4 | 0.65 | 0.044 | 0.084 | | |
| 51 | 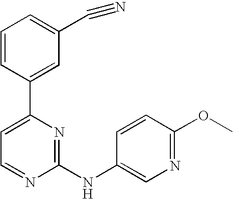 | 3-[2-(6-Methoxy-pyridin- 3-ylamino)-pyrimidin-4- yl]-benzonitrile | | | 7.0 | | 28 | 3.0 | 0.37 | | |
| 52 | 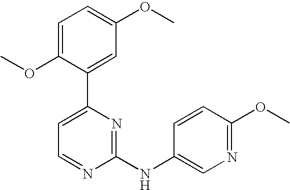 | [4-(2,5-Dimethoxy-phenyl)- pyrimidin-2-yl]-(6-methoxy- pyridin-3-yl)-amine | | | | | | 16 | | | |
| 53 | 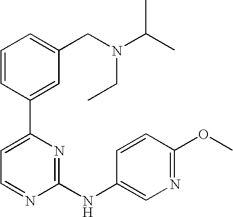 | (4-{3-[(Ethyl-isopropyl- amino)-methyl]-phenyl}- pyrimidin-2-yl)-(6-methoxy- pyridin-3-yl)-amine | 3.9 | 3.4 | 0.35 | 1.1 | 2.5 | 0.028 | 0.051 | | |
| 54 | 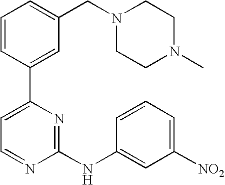 | {4-[3-(4-Methyl-piperazin- 1-ylmethyl)-phenyl]-pyrimidin- 2-yl}-(3-nitro-phenyl)-amine | 2.0 | 0.82 | 0.30 | 4.5 | 0.13 | 0.022 | 0.32 | | |
| 55 | 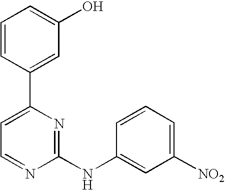 | 3-[2-(3-Nitro-phenyl- amino)-pyrimidin-4-yl]-phenol | | | 0.04 | | 0.31 | 0.03 | 0.003 | | |
| 56 | 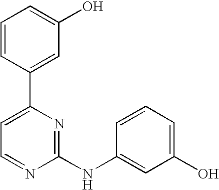 | [3-[2-(3-Hydroxy-phenyl- amino)-pyrimidin-4-yl]-phenol | | | 0.24 | | 2.1 | 0.07 | 0.07 | | |
| 57 | 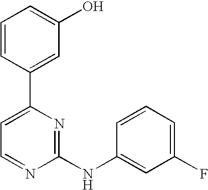 | 3-[2-(3-Fluoro-phenyl- amino)-pyrimidin-4-yl]-phenol | | | 0.21 | | 1.3 | 0.18 | 0.054 | | |
| 58 | 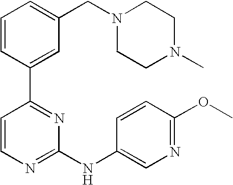 | (6-Methoxy-pyridin-3-yl)- {4-[3-(4-methyl-piperazin- 1-ylmethyl)-phenyl]-pyrimidin-2- yl}-amine | | 10 | | 11 | 36 | 0.19 | | | |
| 59 | 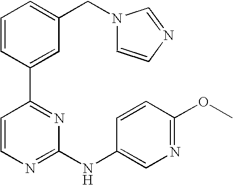 | [4-(3-Imidazol-1-ylmethyl- phenyl)-pyrimidin-2-yl]-(6- methoxy-pyridin-3-yl)-amine | 0.12 | 0.06 | 1.3 | 3.8 | 0.50 | 0.04 | 0.07 | | |
| 60 | 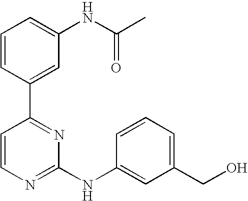 | N-{3-[2-(3-Hydroxymethyl- phenylamino)-pyrimidin-4- yl]-phenyl}-acetamide | | | 1.6 | | 3.8 | 0.26 | 0.51 | | |
| 61 | 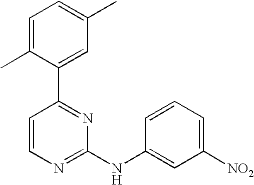 | [4-(2,5-Dimethyl-phenyl)- pyrimidin-2-yl]-(3-nitro- phenyl)-amine | | | 1.3 | | | | 0.57 | | |
| 62 | 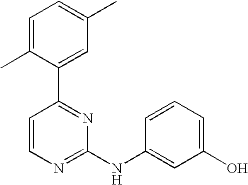 | 3-[4-(2,5-Dimethyl-phenyl)- pyrimidin-2-ylamino]-phenol | | | 1.8 | | | | | | |
| 63 | 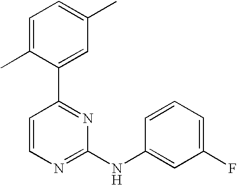 | [4-(2,5-Dimethyl-phenyl)- pyrimidin-2-yl]-(3- fluoro-phenyl)-amine | | | | | | | | | |
| 64 | 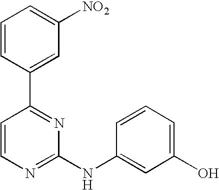 | 3-[4-(3-Nitro-phenyl)- pyrimidin-2-ylamino]-phenol | | | 0.70 | | | | 0.06 | | |
| 65 | 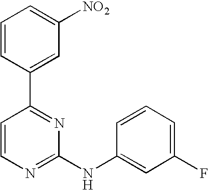 | (3-Fluoro-phenyl)-[4-(3-nitro- phenyl)-pyrimidin-2-yl]-amine | | | | | | | | | |
| 66 | 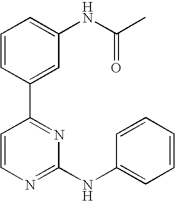 | N-[3-(2-Phenylamino- pyrimidin-4-yl)-phenyl]-acetamide | | | 2.8 | | | | 0.63 | | |
| 67 | 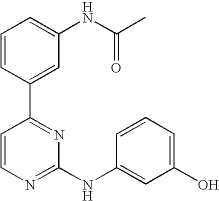 | N-{3-[2-(3-Hydroxy-phenyl- amino)-pyrimidin-4-yl]- phenyl}-acetamide | | | 0.78 | | 4.4 | 0.16 | 0.15 | | |
| 68 | 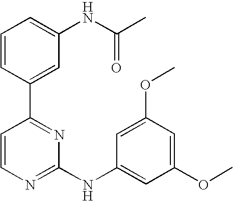 | N-{3-[2-(3,5-Dimethoxy- phenylamino)-pyrimidin-4- yl]-phenyl}-acetamide | | | 2.7 | | | | | | |
| 69 | 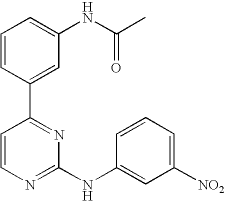 | N-{3-[2-(3-Nitro-phenyl- amino)-pyrimidin-4-yl]- phenyl}-acetamide | | | 0.31 | | 0.55 | 0.04 | 0.14 | | |
| 70 | 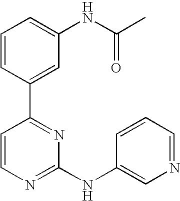 | N-{3-[2-(Pyridin-3-ylamino)- pyrimidin-4-yl]-phenyl}-acetamide | | | | | | | | | |
| 71 | 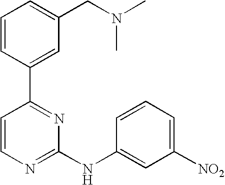 | [4-(3-Dimethylaminomethyl- phenyl)-pyrimidin-2-yl]- (3-nitro-phenyl)-amine | 0.01 | 0.03 | 0.01 | 0.20 | 0.03 | 0.003 | 0.03 | | |
| 72 | 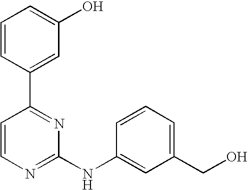 | 3-[2-(3-Hydroxymethyl- phenylamino)-pyrimidin-4-yl]-phenol | 0.15 | 0.15 | 0.12 | 0.28 | 1.7 | | 0.03 | | 0.04 |
| 73 | 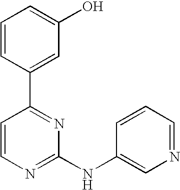 | 3-[2-(Pyridin-3-ylamino)- pyrimidin-4-yl]-phenol | | | | | | | | | |
| 74 | 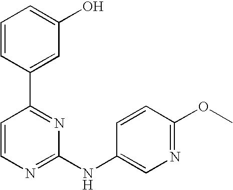 | 3-[2-(6-Methoxy-pyridin-3- ylamino)-pyrimidin-4-yl]-phenol | | | | | 3.8 | | 0.05 | | |
| 75 | 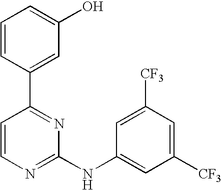 | 3-[2-(3,5-Bis-trifluoromethyl- phenylamino)-pyrimidin-4-yl]-phenol | | | | | | | | | |
| 76 | 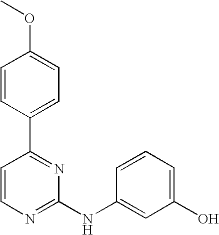 | 3-[4-(4-Methoxy-phenyl)- pyrimidin-2-ylamino]-phenol | 0.001 | <0.001 | 0.01 | 0.01 | 0.12 | <0.001 | 0.01 | | 0.05 |
| 77 | 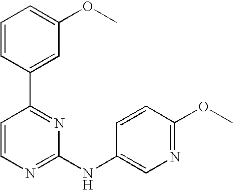 | [4-(3-Methoxy-phenyl)- pyrimidin-2-yl]-(6-methoxy- pyridin-3-yl)-amine | | | | | | | | | |
| 78 | 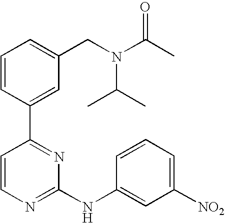 | N-Isopropyl-N-{3-[2-(3- nitro-phenylamino)-pyrimidin- 4-yl]-benzyl}-acetamide | | | | | | | | | |
| 79 | 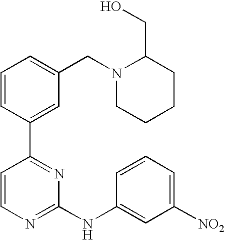 | (1-{3-[2-(3-Nitro-phenyl- amino)-pyrimidin-4-yl]-benzyl}- piperidin-2-yl)-methanol | 0.15 | 0.15 | <0.001 | 0.54 | 0.05 | 0.01 | 0.02 | | |
| 80 | 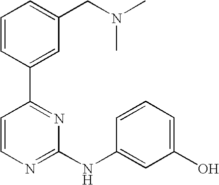 | 3-[4-(3-Dimethylaminomethyl- phenyl)-pyrimidin-2-ylamino]-phenol | 0.69 | 0.59 | 0.07 | | 0.72 | 0.01 | 0.83 | | 1.5 |
| 81 | 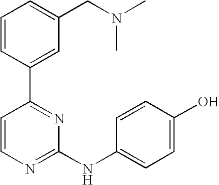 | 4-[4-(3-Dimethylaminomethyl- phenyl)-pyrimidin-2-ylamino]-phenol | 1.3 | 0.50 | 0.11 | | 1.4 | 0.02 | 1.3 | | 1.0 |
| 82 | 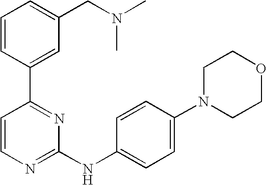 | [4-(3-Dimethylaminomethyl- phenyl)-pyrimidin-2-yl]-(4- morpholin-4-yl-phenyl)-amine | 1.1 | 1.5 | 0.66 | 4.6 | 2.9 | 0.33 | 2.3 | | 0.40 |
| 83 | 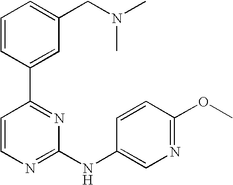 | [4-(3-Dimethylaminomethyl- phenyl)-pyrimidin-2-yl]-(6- methoxy-pyridin-3-yl)-amine | 0.38 | 0.65 | 0.19 | | 0.99 | 0.02 | 0.15 | | 7.8 |
| 84 | 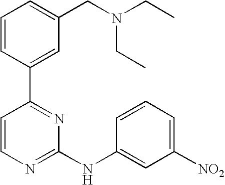 | [4-(3-Diethylaminomethyl- phenyl)-pyrimidin-2-yl]-(3- nitro-phenyl)-amine | 0.02 | 0.02 | 0.06 | 3.2 | 0.09 | 0.004 | 0.02 | | 0.57 |
| 85 | 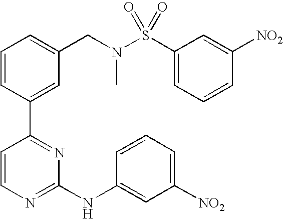 | N-Methyl-3-nitro-N-{3-[2-(3- nitro-phenylamino)-pyrimidin- 4-yl]-benzyl}-benzenesulfonamide | | | | | | 0.40 | | | |
| 86 | 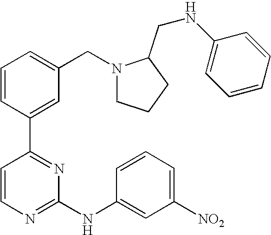 | (3-Nitro-phenyl)-{4-[3-(2- phenylaminomethyl-pyrrolidin-1- ylmethyl)-phenyl]-pyrimidin- 2-yl}-amine | | | 0.81 | | 0.67 | 0.13 | 0.16 | | 2.4 |
| 87 | 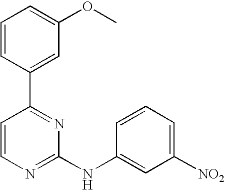 | [4-(3-Methoxy-phenyl)- pyrimidin-2-yl]-(3-nitro- phenyl)-amine | | | | | | 0.003 | 0.07 | | |
| 88 | 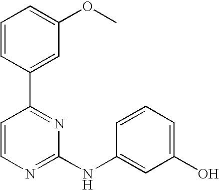 | 3-[4-(3-Methoxy-phenyl)- pyrimidin-2-ylamino]-phenol | 0.57 | 1.4 | 0.10 | 0.20 | | 0.005 | 0.08 | | 0.02 |
| 89 | 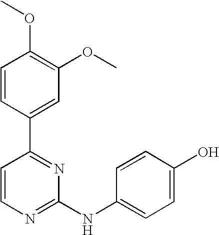 | 4-[4-(3,4-Dimethoxy-phenyl)- pyrimidin-2-ylamino]-phenol | | | | | | | | | |
| 90 | 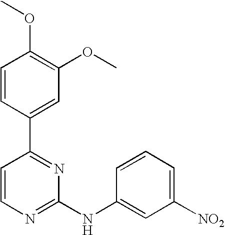 | [4-(3,4-Dimethoxy-phenyl)- pyrimidin-2-yl]-(3- nitro-phenyl)-amine | | | | | | | | | |
| 91 | 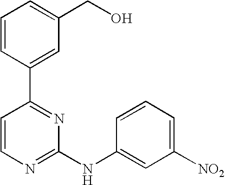 | {3-[2-(3-Nitro-phenyl- amino)-pyrimidin-4-yl]- phenyl}-methanol | 0.59 | 0.47 | 0.06 | 0.87 | 0.55 | 0.0001 | 0.0007 | | 0.16 |
| 92 | 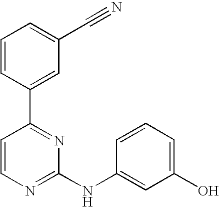 | 3-[2-(3-Hydroxy-phenylamino)- pyrimidin-4-yl]-benzonitrile | 2.1 | | | 1.4 | | 0.11 | 0.08 | | 0.94 |
| 93 | 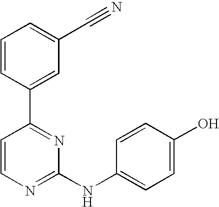 | 3-[2-(4-Hydroxy-phenylamimo)- pyrimidin-4-yl]-benzonitrile | 3.1 | | | 2.5 | | | | | 1.0 |
| 94 | 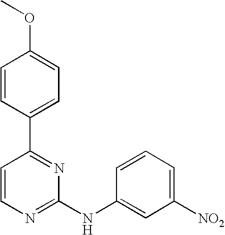 | [4-(4-Methoxy-phenyl)- pyrimidin-2-yl]-(3-nitro- phenyl)-amine | | 0.93 | 0.30 | | 0.99 | 0.02 | 0.03 | | |
| 95 | 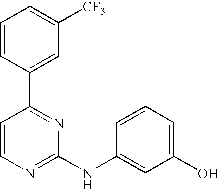 | 3-[4-(3-Trifluoromethyl-phenyl)- pyrimidin-2-ylamino]-phenol | | | | | | 0.09 | | | 0.99 |
| 96 | 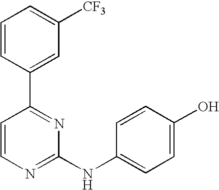 | 4-[4-(3-Trifluoromethyl- phenyl)-pyrimidin-2-ylamino]-phenol | | | | | | 0.08 | | | 0.02 |
| 97 | 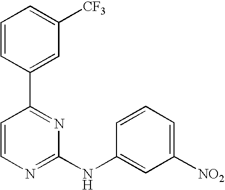 | (3-Nitro-phenyl)-[4-(3- trifluoromethyl-phenyl)- pyrimidin-2-yl]-amine | | | | | | 0.16 | 0.27 | | |
| 98 | 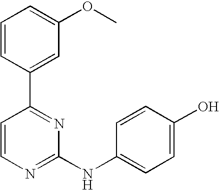 | 4-[4-(3-Methoxy-phenyl)- pyrimidin-2-ylamino]-phenol | 1.1 | 1.4 | 0.13 | 0.59 | | 0.006 | 0.35 | | 0.64 |
| 99 | 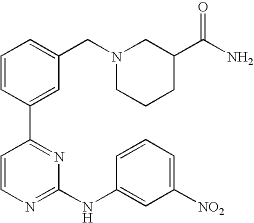 | 1-{3-[2-(3-Nitro-phenyl- amino)-pyrimidin-4-yl]- benzyl}-piperidine-3- carboxylic acid amide | 1.2 | 1.7 | 0.20 | 3.8 | 0.18 | 0.001 | 0.04 | | 1.0 |
| 100 | 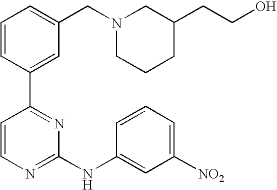 | 2-(1-{3-[2-(3-Nitro- phenylamino)-pyrimidin-4-yl]- benzyl}-piperidin-3-yl)-ethanol | 1.2 | 0.58 | 0.06 | 3.4 | 0.07 | 0.002 | 0.02 | | |
| 101 | 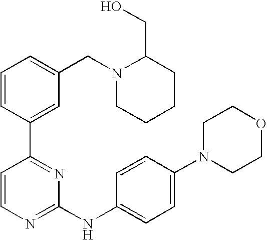 | (1-{3-[2-(4-Morpholin- 4-yl-phenylamino)-pyrimidin-4- yl]-benzyl-piperidin-2- yl)-methanol | | | | | 1.6 | 2.9 | | | |
| 102 | 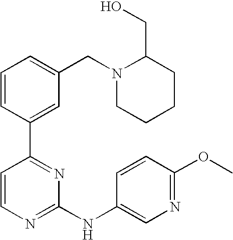 | (1-{3-[2-(6-Methoxy- pyridin-3-ylamino)-pyrimidin- 4-yl]-benzyl}-piperidin-2- yl)-methanol | | | | | 1.4 | 0.49 | | | |
| 103 | 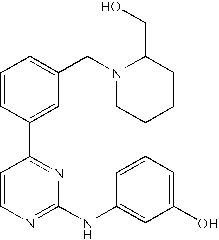 | 3-{4-[3-(2-Hydroxymethyl- piperidin-1-ylmethyl)-phenyl]- pyrimidin-2-ylamino}-phenol | | | | | 0.64 | 0.14 | | | |
| 104 | 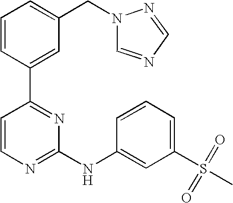 | (3-Methanesulfonyl-phenyl)- [4-(3-[1,2,4]triazol-1- ylmethyl-phenyl)-pyrimidin-2-yl]-amine | | | | | 6.4 | 0.06 | 0.28 | | 1.5 |
| 105 | 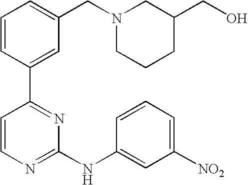 | (1-{3-[2-(3-Nitro- phenylamino)-pyrimidin-4-yl]- benzyl}-piperidin-3-yl)-methanol | 1.2 | 0.35 | 0.07 | 1.9 | 0.08 | 0.01 | 0.01 | | 2.6 |
| 106 | 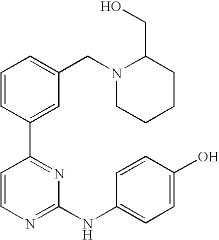 | 4-{4-[3-(2-Hydroxymethyl- piperidin-1-ylmethyl)-phenyl]- pyrimidin-2-ylamino}-phenol | | | | | 1.7 | 0.19 | | | |
| 107 | 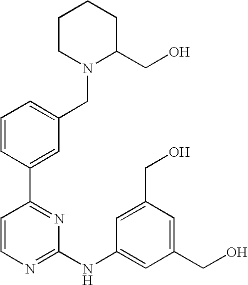 | (1-{3-[2-(3,5-Bis-hydroxy- methyl-phenylamino)-pyrimidin- 4-yl]-benzyl}-pipendin- 2-yl)-methanol | | | | | 1.4 | 0.65 | | | |
| 108 | 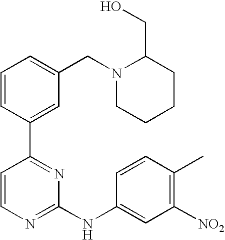 | (1-{3-[2-(4-Methyl-3-nitro- phenylamino)-pyrimidin-4-yl]- benzyl}-piperidin-2-yl)-methanol | 1.1 | 0.44 | 0.06 | 1.7 | 0.03 | <0.001 | 0.02 | | |
| 109 | 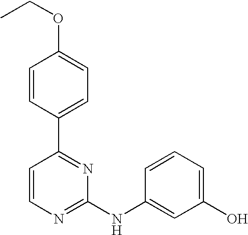 | 3-[4-(4-Ethoxy-phenyl)- pyrimidin-2-ylamino]-phenol | | 2.0 | | | | 0.14 | | | 0.70 |
| 110 | 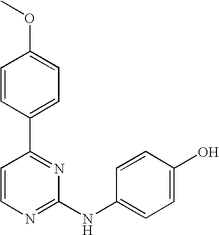 | 4-[4-(4-Methoxy-phenyl)- pyrimidin-2-ylamino]-phenol | 0.50 | 0.08 | 0.12 | 1.3 | 1.5 | 0.02 | | | 0.40 |
| 111 | 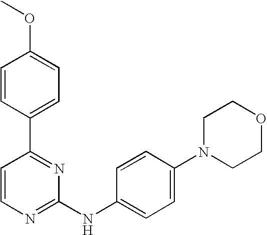 | [4-(4-Methoxy-phenyl)- pyrimidin-2-yl]-(4-morpholin- 4-yl-phenyl)-amine | | | | | | | | | 0.52 |
| 112 | 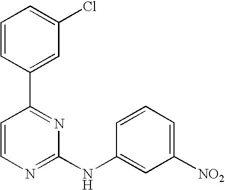 | [4-(3-Chloro-phenyl)-pyrimidin- 2-yl]-(3-nitro-phenyl)-amine | | | | | | 0.07 | 016 | | 2.6 |
| 113 | 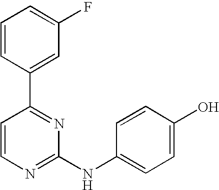 | 4-[4-(3-Fluoro-phenyl)- pyrimidin-2-ylamino]-phenol | | | | | | 0.15 | | | 0.14 |
| 114 | 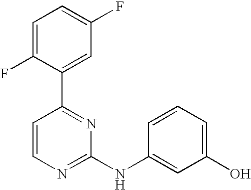 | 3-[4-(2,5-Difluoro-phenyl)- pyrimidin-2-ylamino]-phenol | | | | | | 0.12 | | | 0.16 |
| 115 | 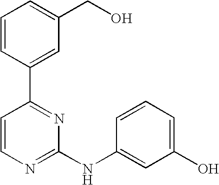 | 3-[4-(3-Hydroxymethyl- phenyl)-pyrimidin-2-ylamino]-phenol | | 2.0 | | | 3.1 | 0.04 | 0.07 | | 0.72 |
| 116 | 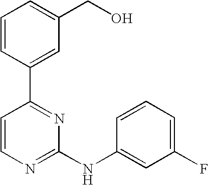 | {3-[2-(3-Fluoro-phenyl- amino)-pyrimidin-4-yl]- phenyl}-methanol | | | | | | 0.05 | | | 0.63 |
| 117 | 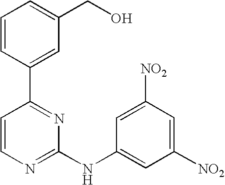 | {3-[2-(3,5-Dinitro-phenylamino)- pyrimidin-4-yl]-phenyl}-methanol | | | | | | | | | |
| 118 | 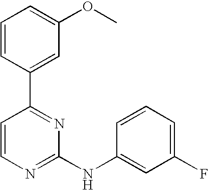 | (3-Fluoro-phenyl)-[4-(3-methoxy- phenyl)-pyrimidin-2-yl]-amine | | | | | | 0.02 | | | 1.8 |
| 119 | 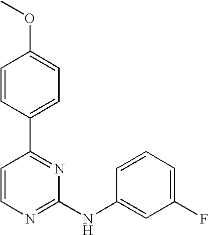 | (3-Fluoro-phenyl)-[4-(4-methoxy- phenyl)-pyrimidin-2-yl]-amine | | | | | | 0.04 | 0.14 | | |
| 121 | 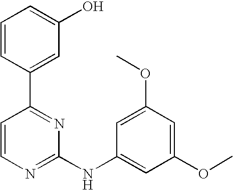 | 3-[2-(3,5-Dimethoxy-phenylamino)- pyrimidin-4-yl]-phenol | | | | | | | | | |
| 122 | 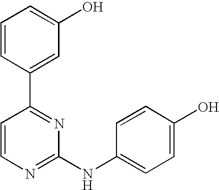 | 3-[2-(4-Hydroxy-phenylamino)- pyrimidin-4-yl]-phenol | | | | | | 0.19 | | | 0.006 |
| 123 | 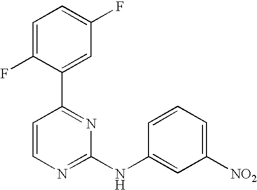 | [4-(2,5-Difluoro-phenyl)-pyrimidin-2- yl]-(3-nitro-phenyl)-amine | | | | | | 0.17 | | | |
| 124 | 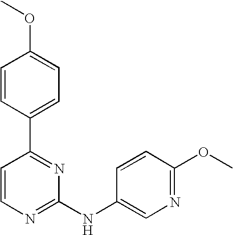 | [4-(4-Methoxy-phenyl)-pyrimidin-2- yl]-(6-methoxy-pyridin-3-yl)-amine | | 1.1 | | 1.5 | | 0.02 | 0.09 | | |
| 125 | 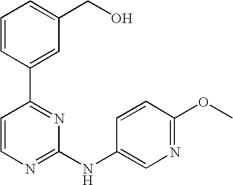 | {3-[2-(6-Methoxy-pyridin-3-ylamino)- pyrimidin-4-yl]-phenyl}-methanol | | | | | 5.9 | 0.12 | 0.04 | | 0.96 |
| 126 | 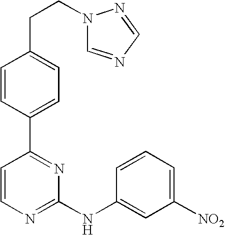 | (3-Nitro-phenyl)-{4-[4-(2-[1,2,4]- triazol-1-yl-ethyl)-phenyl]-pyrimidin- 2-yl}-amine | | | | | | 0.09 | 0.01 | | 0.34 |
| 127 | 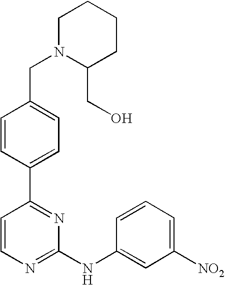 | (1-{4-[2-(3-Nitro-phenylamino)- pyrimidin-4-yl]-benzyl}- piperidin-2-yl)-methanol | | | | | | 0.90 | | | 1.1 |
| 129 | 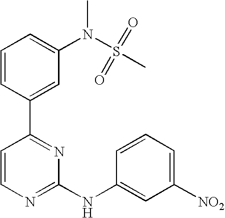 | N-Methyl-N-{3-[2-(3-nitro- phenylamino)-pyrimidin-4-yl]-phenyl}- methanesulfonamide | 0.83 | 0.44 | 0.07 | 2.6 | 0.65 | 0.02 | 0.13 | | 0.27 |
| 130 | 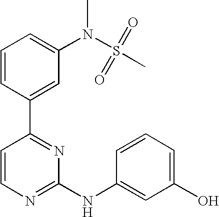 | N-{3-[2-(3-Hydroxy-phenylamino)- pyrimidin-4-yl]-phenyl}-N-methyl- methanesulfonamide | 0.96 | 0.62 | 0.22 | 1.3 | 1.3 | 0.004 | 0.05 | | 0.12 |
| 131 | 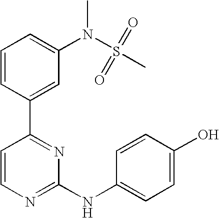 | N-{3-[2-(4-Hydroxy-phenylamino)- pyrimidin-4-yl]-phenyl}-N-methyl- methanesulfonamide | 2.1 | 0.45 | 0.18 | 2.5 | 1.4 | 0.01 | 0.13 | | 0.14 |
| 132 | 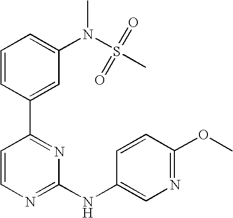 | N-{3-[2-(6-Methoxy-pyridin-3- ylamino)-pyrimidin-4-yl]-phenyl}-N- methyl-methanesulfonamide | | | | 2.1 | 3.9 | 0.06 | 0.17 | | 0.39 |

TABLE 2
Anti-proliferative activity of selected compounds against transformed
human cell lines in vitro.

72-h MTT IC₅₀ (μM)

| Compound | Cell line | | | |
|---|---|---|---|---|
| No. | A549 | HT29 | Saos-2 | Average |
| 1 | 14.5 | 22.9 | 44.2 | 27.2 ± 15.3 |
| 3 | 1.8 | 3.6 | 5.2 | 3.5 ± 1.7 |
| 10 | 8.0 | 9.7 | 5.4 | 7.7 ± 2.2 |
| 11 | 8.3 | 7.5 | 7.1 | 7.6 ± 0.6 |
| 20 | 35.5 | 35.8 | 24.7 | 32.0 ± 6.3 |
| 21 | 11.7 | 15.1 | 41.5 | 22.8 ± 16.3 |
| 22 | 43.5 | 85.2 | 100 | 76.2 ± 29.3 |
| 23 | 12.9 | 3.4 | 23 | 13.1 ± 9.8 |
| 24 | 100 | 7.8 | 80.4 | 62.7 ± 48.6 |
| 26 | 1.7 | 1.2 | 1.1 | 1.3 ± 0.3 |
| 27 | 14.1 | 4.9 | 43.8 | 20.9 ± 20.3 |
| 28 | 8.0 | 16.0 | 7.1 | 10.4 ± 4.9 |
| 29 | 3.6 | 1.8 | 4.5 | 3.3 ± 1.4 |
| 30 | 8.0 | 7.1 | 4.4 | 6.5 ± 1.8 |
| 32 | 19.7 | 6.1 | 40.3 | 22.0 ± 17.2 |
| 33 | 10.3 | 20.7 | 7.4 | 12.8 ± 7.0 |
| 34 | 4.1 | 8.3 | 3.7 | 5.4 ± 2.5 |
| 35 | 15.0 | 7.7 | 23.2 | 15.3 ± 7.7 |
| 37 | 22 | 15.2 | 60.7 | 32.6 ± 24.5 |
| 39 | 6.8 | 2 | 8 | 5.6 ± 3.2 |
| 40 | 1.6 | 1.2 | 4.3 | 2.4 ± 1.7 |
| 42 | 10.6 | 6.7 | 25.8 | 14.4 ± 10.1 |
| 43 | 14.9 | 7.8 | 30.9 | 17.9 ± 11.8 |
| 44 | 1.5 | 1.3 | 1 | 1.3 ± 0.3 |
| 45 | 25.9 | 8.1 | 17.4 | 17.1 ± 8.9 |
| 46 | 0.96 | 0.53 | 1.1 | 0.9 ± 0.3 |
| 48 | 13.4 | 5.1 | 20.4 | 13.0 ± 7.7 |
| 50 | 14.3 | 11.9 | 33.8 | 20.0 ± 12.0 |
| 51 | 35.7 | 10.3 | 67 | 37.7 ± 28.4 |
| 52 | 20.5 | 10.8 | 14.9 | 15.4 ± 4.9 |
| 53 | 2.2 | 0.85 | 3.2 | 2.1 ± 1.2 |
| 54 | 1.1 | 0.77 | 1.3 | 1.1 ± 0.3 |
| 58 | 7 | 5.7 | 9.6 | 7.4 ± 2.0 |
| 78 | 3.7 | 0.96 | 5.4 | 3.4 ± 2.2 |
| 79 | 0.33 | 0.2 | 0.62 | 0.4 ± 0.2 |
| 80 | 0.86 | 1.6 | 4 | 2.2 ± 1.6 |
| 81 | 1.5 | 1.2 | 5 | 2.6 ± 2.1 |
| 82 | 6.2 | 4.5 | 6.6 | 5.8 ± 1.1 |
| 83 | 1.4 | 0.91 | 1.6 | 1.3 ± 0.4 |
| 85 | 76.9 | 10.4 | 44.6 | 44.0 ± 33.3 |
| 86 | 49.3 | 8.9 | 55.8 | 38.0 ± 25.4 |
| 99 | 3 | 2.6 | 4.1 | 3.2 ± 0.8 |
| 100 | 0.56 | 0.64 | 1.3 | 0.8 ± 0.4 |
| 101 | 17.3 | 6 | 25 | 16.1 ± 9.6 |
| 102 | 17 | 11.1 | 23.4 | 17.2 ± 6.2 |
| 103 | 6.9 | 7 | 5.6 | 6.5 ± 0.8 |

### REFERENCES

1. Manning, G.; Whyte, D. B.; Martinez, R.; Hunter, T.; Sudarsanam, S. The protein kinase complement of the human genome. Science 2002, 298, 1912-1934.
2. Kostich, M.; English, J.; Madison, V.; Gheyas, F.; Wang, L. et al. Human members of the eukaryotic protein kinase family. Genome Biology 2002, 3, research0043.0041-0043.0012.
3. Dancey, J.; Sausville, E. A. Issues and progress with protein kinase inhibitors for cancer treatment. Nat. Rev. Drug Disc. 2003, 2, 296-313.
4. Cockerill, G. S.; Lackey, K. E. Small molecule inhibitors of the class 1 receptor tyrosine kinase family. Current Topics in Medicinal Chemistry 2002, 2, 1001-1010.
5. Fabbro, D.; Ruetz, S.; Buchdunger, E.; Cowan-Jacob, S. W.; Fendrich, G. et al. Protein kinases as targets for anticancer agents: from inhibitors to useful drugs. Pharmacol.Ther. 2002, 93, 79-98.
6. Cohen, P. Protein kinases—the major drug targets of the twenty-first century? Nat. Rev. Drug Disc. 2002, 1, 309-315.
7. Bridges, A. J. Chemical inhibitors of protein kinases. Chem.Rev. 2001, 101(8), 2541-2571.
8. Wang, S.; Meades, C.; Wood, G.; Osnowski, A.; Fischer, P. M. N-(4-(4-methylthiazol-5-yl) pyrimidin-2-yl)-N-phenylamines as antiproliferative compounds. PCT Intl. Patent Appl. Publ. WO 2003029248; Cyclacel Limited, UK.
9. Wu, S. Y.; McNae, I.; Kontopidis, G.; McClue, S. J.; McInnes, C. et al. Discovery of a Novel Family of CDK Inhibitors with the Program LIDAEUS: Structural Basis for Ligand-Induced Disordering of the Activation Loop. Structure 2003, 11, 399-410.
10. Fischer, P. M.; Wang, S.; Wood, G. Inhibitors of cyclin dependent kinases as anti-cancer agent. PCT Intl. Patent Appl. Publ. WO 02/079193; Cyclacel Limited, UK,.
11. Wang, S.; Fischer, P. M. Anti-cancer compounds. US Patent Appl. Publ. 2002/0019404.
12. Fischer, P. M.; Wang, S. 2-substituted 4-heteroaryl-pyrimidines and their use in the treatmetn of proliferative disorders. PCT Intl. Patent Appl. Publ. WO 2001072745; Cyclacel Limited, UK.
13. Knockaert, M.; Greengard, P.; Meijer, L. Pharmacological inhibitors of cyclin-dependent kinases. Trends Pharmacol. Sci. 2002, 23, 417-425.
14. Fischer, P. M.; Endicott, J.; Meijer, L. Cyclin-dependent kinase inhibitors. Progress in Cell Cycle Research; Editions de la Station Biologique de Roscoff: Roscoff, France, 2003; pp 235-248.
15. Fravolini, A.; Grandolini, G.; Martani, A. New heterocyclic ring systems from α-hydroxymethylene ketones. V. Reaction of 2-methyl-6-hydroxymethylene-4,5,6,7-tetrahydrobenzothiazol-7-one with amines and amidines. Gazz. Chim. Ital. 1973, 103, 1063-1071.
16. Cleaver, L.; Croft, J. A.; Ritchie, E.; Taylor, W. C. Chemical studies of the Proteaceae. IX. Synthesis of 5-alkylresorcinols from aliphatic precursors. Aust. J. Chem. 1976, 29, 1989-2001.
17. Fadda, A. A.; El-Houssini, M. S. Synthesis of cyclic ketones by activated nitriles. J. Ind. Chem. Soc. 1990, 67, 915-917.
18. Kost, A. N.; Ovseneva, L. G. Synthesis of 4-substituted dihydroresorcinols. Zh. Obshch. Khim 1962, 32, 3983-3986.
19. Lehmann, G.; Luecke, B.; Schick, H.; Hilgetag, G. 2-Substituted 7-oxo-4,5,6,7-tetrahydrobenzothiazoles. Z. Chem. 1967, 7, 422.
20. Bell, R. P.; Davis, G. G. Kinetics of the bromination of some enols and their anions. J. Chem. Soc 1965, 353-361.
21. Fravolini, A.; Grandolini, G.; Martani, A. New heterocyclic ring systems from α-hydroxymethylene ketones. III. Pyrazolobenzothiazoles and thiazolo-benzoisoxazoles. Gazz. Chim. Ital. 1973, 103, 755-769.
22. Bredereck, H.; Effenberger, F.; Botsch, H. Acid amide reactions. XLV. Reactivity of formamidines, dimethylformamide diethyl acetal (amide acetal), and bis(dimethylamino)ₘethoxymethane (aminal ester). Chem. Ber. 1964, 97, 3397-3406.
23. Wang D, De la Fuente C, Deng L, Wang L, Zilberman I, Eadie C, Healey M, Stein D, Denny T, Harrison L E, Meijer L, Kashanchi F. Inhibition of human immunodeficiency virus type 1 transcription by chemical cyclin-dependent kinase inhibitors. J. Virol. 2001; 75: 7266-7279.
24. Chen, Y. H.; Hansen, L.; Chen, M. X.; Bjorbaek, C.; Vestergaard, H.; Hansen, T.; Cohen, P. T.; Pedersen, O. Diabetes, 1994, 43, 1234.
25. Nikoulina, S. E.; Ciaraldi, T. P.; Mudaliar, S.; Mohideen, P.; Carter, L.; Henry, R. R. Diabetes, 2000, 49, 263.
26. Goedert, M. Curr. Opin. Gen. Dev., 2001, 11, 343.
27. Mattson, M. P. Nat. Rev. Mol. Cell. Biol., 2000, 1, 120.
28. Zhu, A. J.; Watt, F. M. Development, 1999, 126, 2285.
29. DasGupta, R.; Fuchs, E. Development, 1999, 126, 4557.
30. Sunkel et al., J. Cell Sci., 1988, 89, 25.
31. Llamazares et al., Genes Dev., 1991, 5, 2153.
32. Glover et al., Genes Dev., 1998, 12, 3777.
33. Lee et al., Proc. Natl. Acad. Sci. USA, 1998, 95, 9301.
34. Leung et al., Nat. Struct. Biol., 2002, 9, 719.
35. Kauselmann et al., EMBO J., 1999, 18, 5528.
36. Nigg, Curr. Opin. Cell Biol., 1998, 10, 776.
37. Yuan et al., Cancer Res., 2002, 62, 4186.
38. Seong et al., J. Biol. Chem., 2002, 277, 32282.
39. Lane et al., J. Cell. Biol., 1996, 135, 1701.
40. Cogswell et al., Cell Growth Differ., 2000, 11, 615.
41. Liu et al., Proc. Natl. Acad. Sci. USA, 2002, 99, 8672.
42. Toyoshima-Morimoto et al., Nature, 2001, 410, 215.
43. Roshak et al., Cell. Signalling, 2000, 12, 405.
44. Smits et al., Nat. Cell Biol., 2000, 2, 672.
45. van Vugt et al., J. Biol. Chem., 2001, 276, 41656.
46. Sumara et al., Mol. Cell, 2002, 9, 515.
47. Golan et al., J. Biol. Chem., 2002, 277, 15552.
48. Kotani et al., Mol. Cell, 1998, 1, 371.
49. Feng et al., Cell Growth Differ., 2001, 12, 29.
50. Dai et al., Oncogene, 2002, 21, 6195.
51. Nurse, Nature, 1990, 344, 503.
52. Nigg, Nat. Rev. Mol. Cell Biol., 2001, 2, 21.
53. Hagting et al., EMBO J., 1998, 17, 4127.
54. Hagting et al., Curr. Biol., 1999, 9, 680.
55. Yang et al., J. Biol. Chem., 2001, 276, 3604.
56. Takizawa et al., Curr. Opin. Cell Biol., 2000, 12, 658.
57. Seki et al., Mol. Biol. Cell, 1992, 3, 1373.
58. Heald et al., Cell, 1993, 74, 463.
59. Dalal et al., Mol. Cell. Biol., 1999, 19, 4465.
60. Toyoshima-Morimoto et al., Nature, 2001, 410, 215.
61. Toyoshima-Morimoto et al., EMBO Rep., 2002, 3, 341.
62. Wang et al., Mol. Cell. Biol., 2002, 22, 3450.
63. Tyrrell, E.; Brookes, P. Synthesis 2003, 469-483.
64. Molander, G. A.; Biolatto, B. J. Org. Chem. 2003, 68, 4302-4314.
65. Bredereck, H.; Effenberger, F.; Botsch, H. Chem. Ber. 1964, 97, 3397-3406.
66. Zimmermann, J.; Caravatti, G.; Mett, H.; Meyer, T.; Müiller, M. et al. Arch. Pharm. Pharm. Med. Chem. 1996, 329, 371-376.
67. Haselsberger, K.; Peterson, D. C.; Thomas, D. G.; Darling, J. L. Anti Cancer Drugs 1996, 7, 331-8.
68. Loveland, B. E.; Johns, T. G.; Mackay, I. R.; Vaillant, F.; Wang, Z. X.; Hertzog, P. J. Biochemistry International 1992, 27, 501-10.

1. A compound of formula I, or a pharmaceutically acceptable salt thereof, 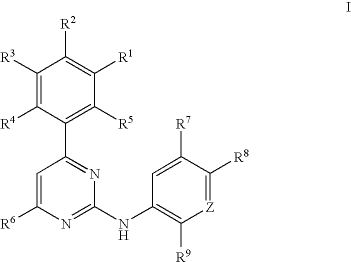
wherein:
Z is CR¹⁰ or N;
one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)ₙR¹¹, (CH₂)ₘCONR¹²R¹³, (CH₂)ₘCO(CH₂)ₙR¹¹ and (CH₂)ₘCOR¹²; where m is 0, 1, 2, 3 or 4 and n is 1, 2, 3 or 4;
the other of R¹ and R² is H or R¹¹;
R³ and R⁵ are both H;
R⁴ is H or R¹¹;
R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹², (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
providing that when
Z is CR¹⁰ and R⁹ is H, at least one of R⁷, R⁸ and R¹⁰ is other than OMe; and
Z is CR¹⁰and R⁷⁻⁹ are all H, R¹⁰ is other than OCF₂CHF₂.

2. A compound according to claim 1 wherein one of R¹ and R² is selected from (CH₂)ₘR¹¹, (CH₂)ₘR¹², (CH₂)ₘNR¹²R¹³, (CH₂)ₘNR¹³COR¹², and (CH₂)ₘOR¹².

3. A compound according to claim 2 wherein R¹ is selected from (CH₂)ₘR¹¹, (CH₂)ₘR¹², (CH₂)ₘNR¹²R¹³, (CH₂)ₘNR¹³COR¹², and (CH₂)ₘOR¹².

4. A compound according to claim 1 wherein one of R¹ and R² is selected from NO₂, CN, halogen, CH₂R¹¹, CH₂R¹², OR¹², NR¹²R¹³, NR¹³COR¹², CH₂NR¹²R¹³, CH₂NHSO₂R¹⁴, CF₃, NR¹³R¹⁴, R¹³, CH₂NR¹³COR¹² and NR¹³SO₂R¹².

5. A compound according to claim 4 wherein R¹ is selected from NO₂, CN, halogen, CH₂R¹¹, CH₂R¹², OR¹², NR¹²R¹³, NR¹³COR¹², CH₂NR¹²R¹³, CH₂NHSO₂R¹⁴, CF₃, NR¹³R¹⁴, R¹³, CH₂NR¹³COR¹² and NR¹³SO₂R¹².

6. A compound according to claim 1 wherein each R¹² is independently selected from alkyl, alkenyl, alkynyl, aralkyl, a cyclic group, a saturated or unsaturated alicyclic group, and an aryl group, each of which may optionally contain one to four heteroatoms selected from O, S, and N, and each of which may optionally be substituted with one, two or three R¹¹ groups.

7. A compound according to claim 1 wherein each R¹³ and each R¹⁴ is independently H or a C₁₋₅alkyl group.

8. A compound according to claim 1 wherein R¹⁵ is a C₁₋₅alkyl group.

9. A compound according to claim 1 wherein each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, NR¹³SO₂R¹², (CH₂)ₛNR¹²R¹³, morpholine, piperidine or piperazine, where q, r and s are each independently 0, 1, 2, 3 or 4.

10. A compound according to claim 1 wherein each R¹¹ is selected from halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholine, piperidine and piperazine.

11. A compound according to claim 2 wherein one of R¹ and R² is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂

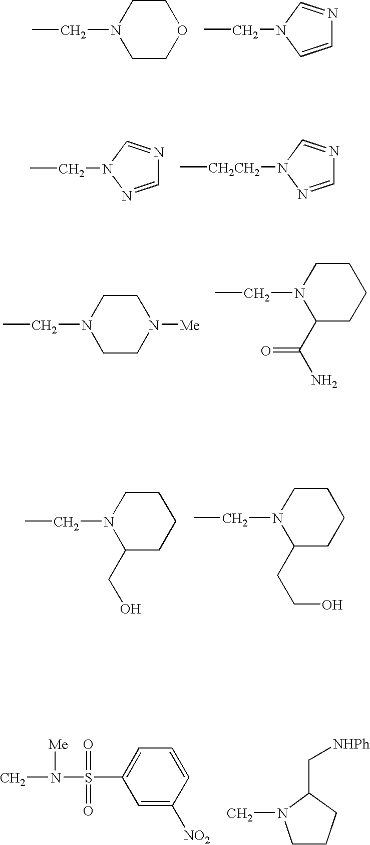

12. A compound according to claim 11 wherein R¹ is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂

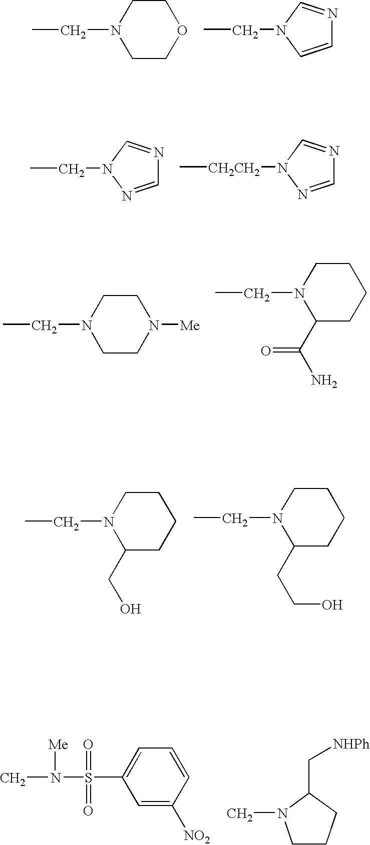

13. A compound according to claim 1 wherein R² is H, halogen, OR¹³ or (CH₂)ₘR¹².

14. A compound according to claim 1 wherein R² is selected from H, Cl, OMe, OEt

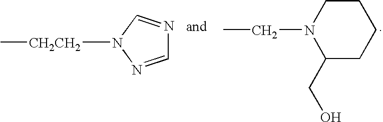

15. A compound according to claim 1 wherein R⁴ is H, OR¹³, halogen or R¹³.

16. A compound according to claim 1 wherein R⁴ is H, OMe, Me or F.

17. A compound according to claim 1 wherein R⁷, R⁸, R⁹, and R¹⁰ are each independently selected from H, halogen, NO₂, CN, OH, NH₂, NHCOMe, CF₃, COMe, Me, Et, ⁱPr, NHMe, NMe₂, CONHMe, CONMe₂, SO₂NH₂, SO₂NHMe, SO₂NMe₂, SO₂Me, OMe, OEt, OCH₂CH₂OH, OCH₂CH₂OMe, CH₂OH, morpholine, piperidine, and piperazine.

18. A compound according to claim 1 wherein R⁶ and R⁹ are both H.

19. A compound according to claim 1 wherein R⁷ is selected from H, NO₂, NR¹³R¹⁴, OR¹³, CN, CF₃, CH₂OR¹³, SO₂R¹³ and halogen.

20. A compound according to claim 1 wherein R⁷ is selected from H, NO₂, NH₂, OH, OMe, CN, CH₂OH, F, CF₃ and SO₂Me.

21. A compound according to claim 1 wherein R⁸ is selected from H, OR¹³, NO₂, OCH₂CH₂OMe, halogen, NR¹³R¹⁴, N-morpholine and OR¹³.

22. A compound according to claim 1 wherein R⁸ is selected from H, OH, NO₂, OCH₂CH₂OMe, Cl, F, NMe₂, N-morpholine, Me and OMe.

23. A compound according to claim 1 wherein Z is CR¹⁰.

24. A compound according to claim 23 wherein R¹⁰ is selected from H, halogen, NO₂, CN, OR¹³, NR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, morpholine, piperidine and piperazine.

25. A compound according to claim 23 wherein R¹⁰ is selected from NO₂, NH₂, H, OH, OMe, CN, F, CH₂OH, CF₂ and SO₂Me.

26. A compound according to claim 23 wherein R¹⁰ is H.

27. A compound according to claim 1 wherein Z is N.

28. A compound, or pharmaceutically acceptable salt thereof, which is selected from the following:
4-[4-(3-Nitro-phenyl)-pyrimidin-2-ylamino]-phenol [1];
(4-Nitro-phenyl)-[4-(3-nitro-phenyl)-pyrimidin-2-yl]-amine [2];
[4-(3-Amino-phenyl)-pyrimidin-2-yl]-[4-(2-methoxy-ethoxy)-phenyl]-amine [3];
[4-(3-Amino-phenyl)-pyrimidin-2-yl]-(4-nitro-phenyl)-amine [4];
(3-Nitro-phenyl)-[4-(3-nitro-phenyl)-pyrimidin-2-yl]-amine [5];
(4-Fluoro-phenyl)-[4-(3-nitro-phenyl)-pyrimidin-2-yl]-amine [6];
[4-(3-Amino-phenyl)-pyrimidin-2-yl]-(4-fluoro-phenyl)-amine [7];
N-[4-(3-Amino-phenyl)-pyrimidin-2-yl]-benzene-1,3-diamine [8];
N,N-Dimethyl-N′-[4-(3-nitro-phenyl)-pyrimidin-2-yl]-benzene-1,4-diamine [9];
N-Ethyl-N-{3-[2-(4-hydroxy-phenylamino)-pyrimidin-4-yl]-phenyl}-acetamide [10];
N-{3-[2-(4-Hydroxy-phenylamino)-pyrimidin-4-yl]-phenyl}-acetamide [11];
N-{3-[2-(4-Hydroxy-phenylamino)-pyrimidin-4-yl]-phenyl}-N-methyl-acetamide [12];
N-{3-[2-(4-Hydroxy-phenylamino)-pyrimidin-4-yl]-phenyl}-N-isobutyl-acetamide [13];
4-[4-(3-Methylamino-phenyl)-pyrimidin-2-ylamino]-phenol [14];
4-[4-(3-Amino-phenyl)-pyrimidin-2-ylamino]-phenol [15];
(4-Chloro-phenyl)-[4-(3-chloro-phenyl)-pyrimidin-2-yl]-amine [16];
4-[4-(3-Chloro-phenyl)-pyrimidin-2-ylamino]-phenol [17];
3-[4-(3-Chloro-phenyl)-pyrimidin-2-ylamino]-phenol [18]
[4-(3-Amino-phenyl)-pyrimidin-2-yl]-(3-nitro-phenyl)-amine [19];
N-[4-(3,4-Dichloro-phenyl)-pyrimidin-2-yl]-N′,N′-dimethyl-benzene-1,4-diamine [20];
4-[4-(3,4-Dichloro-phenyl)-pyrimidin-2-ylamino]-phenol [21];
3-[4-(3,4-Dichloro-phenyl)-pyrimidin-2-ylamino]-phenol [22];
N-Ethyl-N-{3-[2-(4-methoxy-phenylamino)-pyrimidin-4-yl]-phenyl}-acetamide [23];
N-Ethyl-N-{3-[2-(4-nitro-phenylamino)-pyrimidin-4-yl]-phenyl}-acetamide [24];
[4-(3-Ethylamino-phenyl)-pyrimidin-2-yl]-(4-methoxy-phenyl)-amine [25];
[4-(3-Ethylamino-phenyl)-pyrimidin-2-yl]-(4-nitro-phenyl)-amine [26];
{4-[3-(Benzylamino-methyl)-phenyl]-pyrimidin-2-yl}-(3-nitro-phenyl)-amine [27];
3-{4-[3-(Benzylamino-methyl)-phenyl]-pyrimidin-2-ylamino}-phenol [28];
[4-(3-Imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
[4-(3,4-Dichloro-phenyl)-pyrimidin-2-yl]-(3-nitro-phenyl)-amine [31];
(4-Morpholin-4-yl-phenyl)-[4-(3-[1,2,4]triazol-1-ylmethyl-phenyl)-pyrimidin-2-yl]-amine [32];
4-[4-(3-[1,2,4]triazol-1-ylmethyl-phenyl)-pyrimidin-2-ylamino]-phenol [33];
3-[4-(3-[1,2,4]triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35];
3-[4-(3-[1,2,4]triazol-1-ylmethyl-phenyl)-pyrimidin-2-ylamino]-benzonitrile [36]
Phenyl-(4-phenyl-pyrimidin-2-yl)-amine [37];
[4-(5-Fluoro-2-methoxy-phenyl)-pyrimidin-2-yl]-phenyl-amine [38];
[4-(3-Morpholin-4-ylmethyl-phenyl)-pyrimidin-2-yl]-(3-nitro-phenyl)-amine [39];
N-{3-[2-(3-Nitro-phenylamino)-pyrimidin-4-yl]-benzyl}-methanesulfonamide [40];
(4-Nitro-phenyl)-[4-(3-[1,2,4]triazol-1-ylmethyl-phenyl)-pyrimidin-2-yl]-amine [41];
(4-Methoxy-phenyl)-[4-(3-[1,2,4]triazol-1-ylmethyl-phenyl)-pyrimidin-2-yl]-amine [42];
N,N-Dimethyl-N′-[4-(3-[1,2,4]triazol-1-ylmethyl-phenyl)-pyrimidin-2-yl]-benzene-1,4-diamine [43];
[4-(2,5-Dimethoxy-phenyl)-pyrimidin-2-yl]-(3-nitro-phenyl)-amine [44];
4-[4-(2,5-Dimethoxy-phenyl)-pyrimidin-2-ylamino]-phenol [45];
(4-{3-[(Ethyl-isopropyl-amino)-methyl]-phenyl}-pyrimidin-2-yl)-(3-nitro-phenyl)-amine [46];
[4-(4-Chloro-3-[1,2,4]triazol-1-ylmethyl-phenyl)-pyrimidin-2-yl]-(3-nitro-phenyl)-amine [47];
{4-[3-(Benzylamino-methyl)-phenyl]-pyrimidin-2-yl}-(6-chloro-pyridin-3-yl)-amine [48];
[4-(3,4-Dichloro-phenyl)-pyrimidin-2-yl]-(6-methoxy-pyridin-3-yl)-amine [49];
(6-Methoxy-pyridin-3-yl)-[4-(3-[1,2,4]triazol-1-ylmethyl-phenyl)-pyrimidin-2-yl]-amine [50];
3-[2-(6-Methoxy-pyridin-3-ylamino)-pyrimidin-4-yl]-benzonitrile [51];
[4-(2,5-Dimethoxy-phenyl)-pyrimidin-2-yl]-(6-methoxy-pyridin-3-yl)-amine [52];
(4-{3-[(Ethyl-isopropyl-amino)-methyl]-phenyl}-pyrimidin-2-yl)-(6-methoxy-pyridin-3-yl)-amine [53];
{4-[3-(4-Methyl-piperazin-1-ylmethyl)-phenyl]-pyrimidin-2-yl}-(3-nitro-phenyl)-amine [54];
3-[2-(3-Nitro-phenylamino)-pyrimidin-4-yl]-phenol [55];
[3-[2-(3-Hydroxy-phenylamino)-pyrimidin-4-yl]-phenol [56];
3-[2-(3-Fluoro-phenylamino)-pyrimidin-4-yl]-phenol [57];
(6-Methoxy-pyridin-3-yl)-{4-[3-(4-methyl-piperazin-1-ylmethyl)-phenyl]-pyrimidin-2-yl}-amine [58];
[4-(3-Imidazol-1-ylmethyl-phenyl)-pyrimidin-2-yl]-(6-methoxy-pyridin-3-yl)-amine [59];
N-{3-[2-(3-Hydroxymethyl-phenylamino)-pyrimidin-4-yl]-phenyl}-acetamide [60];
[4-(2,5-Dimethyl-phenyl)-pyrimidin-2-yl]-(3-nitro-phenyl)-amine [61];
3-[4-(2,5-Dimethyl-phenyl)-pyrimidin-2-ylamino]-phenol [62];
[4-(2,5-Dimethyl-phenyl)-pyrimidin-2-yl]-(3-fluoro-phenyl)-amine [63];
3-[4-(3-Nitro-phenyl)-pyrimidin-2-ylamino]-phenol [64];
(3-Fluoro-phenyl)-[4-(3-nitro-phenyl)-pyrimidin-2-yl]-amine [65];
N-[3-(2-Phenylamino-pyrimidin-4-yl)-phenyl]-acetamide [66];
N-{3-[2-(3-Hydroxy-phenylamino)-pyrimidin-4-yl]-phenyl}-acetamide [67];
N-{3-[2-(3,5-Dimethoxy-phenylamino)-pyrimidin-4-yl]-phenyl}-acetamide [68];
N-{3-[2-(3-Nitro-phenylamino)-pyrimidin-4-yl]-phenyl}-acetamide [69];
N-{3-[2-(Pyridin-3-ylamino)-pyrimidin-4-yl]-phenyl}-acetamide [70];
[4-(3-Dimethylaminomethyl-phenyl)-pyrimidin-2-yl]-(3-nitro-phenyl)-amine [71];
3-[2-(3-Hydroxymethyl-phenylamino)-pyrimidin-4-yl]-phenol [72];
3-[2-(Pyridin-3-ylamino)-pyrimidin-4-yl]-phenol [73];
3-[2-(6-Methoxy-pyridin-3-ylamino)-pyrimidin-4-yl]-phenol [74];
3-[2-(3,5-Bis-trifluoromethyl-phenylamino)-pyrimidin-4-yl]-phenol [75];
3-[4-(4-Methoxy-phenyl)-pyrimidin-2-ylamino]-phenol [76];
[4-(3-Methoxy-phenyl)-pyrimidin-2-yl]-(6-methoxy-pyridin-3-yl)-amine [77];
N-Isopropyl-N-{3-[2-(3-nitro-phenylamino)-pyrimidin-4-yl]-benzyl}-acetamide [78];
(1-{3-[2-(3-Nitro-phenylamino)-pyrimidin-4-yl]-benzyl}-piperidin-2-yl)-methanol [79];
3-[4-(3-Dimethylaminomethyl-phenyl)-pyrimidin-2-ylamino]-phenol [80];
4-[4-(3-Dimethylaminomethyl-phenyl)-pyrimidin-2-ylamino]-phenol [81];
[4-(3-Dimethylaminomethyl-phenyl)-pyrimidin-2-yl]-(4-morpholin-4-yl-phenyl)-amine [82];
[4-(3-Dimethylaminomethyl-phenyl)-pyrimidin-2-yl]-(6-methoxy-pyridin-3-yl)-amine [83];
[4-(3-Diethylaminomethyl-phenyl)-pyrimidin-2-yl]-(3-nitro-phenyl)-amine [84];
N-Methyl-3-nitro-N-{3-[2-(3-nitro-phenylamino)-pyrimidin-4-yl]-benzyl}-benzenesulfonamide [85];
(3-Nitro-phenyl)-{4-[3-(2-phenylaminomethyl-pyrrolidin-1-ylmethyl)-phenyl]-pyrimidin-2-yl}-amine [86];
[4-(3-Methoxy-phenyl)-pyrimidin-2-yl]-(3-nitro-phenyl)-amine [87];
3-[4-(3-Methoxy-phenyl)-pyrimidin-2-ylamino]-phenol [88];
4-[4-(3,4-Dimethoxy-phenyl)-pyrimidin-2-ylamino]-phenol [89];
[4-(3,4-Dimethoxy-phenyl)-pyrimidin-2-yl]-(3-nitro-phenyl)-amine [90];
{3-[2-(3-Nitro-phenylamino)-pyrimidin-4-yl]-phenyl}-methanol [91];
3-[2-(3-Hydroxy-phenylamino)-pyrimidin-4-yl]-benzonitrile [92];
3-[2-(4-Hydroxy-phenylamino)-pyrimidin-4-yl]-benzonitrile [93];
[4-(4-Methoxy-phenyl)-pyrimidin-2-yl]-(3-nitro-phenyl)-amine [94];
3-[4-(3-trifluoromethyl-phenyl)-pyrimidin-2-ylamino]-phenol [95];
4-[4-(3-trifluoromethyl-phenyl)-pyrimidin-2-ylamino]-phenol [96];
(3-Nitro-phenyl)-[4-(3-trifluoromethyl-phenyl)-pyrimidin-2-yl]-amine [97];
4-[4-(3-Methoxy-phenyl)-pyrimidin-2-ylamino]-phenol [98];
1-{3-[2-(3-Nitro-phenylamino)-pyrimidin-4-yl]-benzyl}-piperidine-3-carboxylic acid amide [99];
2-(1-{3-[2-(3-Nitro-phenylamino)-pyrimidin-4-yl]-benzyl}-piperidin-3-yl)-ethanol [100];
(1-{3-[2-(4-Morpholin-4-yl-phenylamino)-pyrimidin-4-yl]-benzyl}-piperidin-2-yl)-methanol [101];
(1-{3-[2-(6-Methoxy-pyridin-3-ylamino)-pyrimidin-4-yl]-benzyl}-piperidin-2-yl)-methanol [102];
3-{4-[3-(2-Hydroxymethyl-piperidin-1-ylmethyl)-phenyl]-pyrimidin-2-ylamino}-phenol [103];
(3-Methanesulfonyl-phenyl)-[4-(3-[1,2,4]triazol-1-ylmethyl-phenyl)-pyrimidin-2-yl]-amine [104];
(1-{3-[2-(3-Nitro-phenylamino)-pyrimidin-4-yl]-benzyl}-piperidin-3-yl)-methanol [105];
4-{4-[3-(2-Hydroxymethyl-piperidin-1-ylmethyl)-phenyl]-pyrimidin-2-ylamino}-phenol [106];
(1-{3-[2-(3,5-Bis-hydroxymethyl-phenylamino)-pyrimidin-4-yl]-benzyl}-piperidin-2-yl)-methanol [107];
(1-{3-[2-(4-Methyl-3-nitro-phenylamino)-pyrimidin-4-yl]-benzyl}-piperidin-2-yl)-methanol [108];
3-[4-(4-Ethoxy-phenyl)-pyrimidin-2-ylamino]-phenol [109];
4-[4-(4-Methoxy-phenyl)-pyrimidin-2-ylamino]-phenol [110];
[4-(4-Methoxy-phenyl)-pyrimidin-2-yl]-(4-morpholin-4-yl-phenyl)-amine [111];
[4-(3-Chloro-phenyl)-pyrimidin-2-yl]-(3-nitro-phenyl)-amine [112];
4-[4-(3-Fluoro-phenyl)-pyrimidin-2-ylamino]-phenol [113];
3-[4-(2,5-Difluoro-phenyl)-pyrimidin-2-ylamino]-phenol [114];
3-[4-(3-Hydroxymethyl-phenyl)-pyrimidin-2-ylamino]-phenol [115];
{3-[2-(3-Fluoro-phenylamino)-pyrimidin-4-yl]-phenyl}-methanol [116];
{3-[2-(3,5-Dinitro-phenylamino)-pyrimidin-4-yl]-phenyl}-methanol [117];
(3-Fluoro-phenyl)-[4-(3-methoxy-phenyl)-pyrimidin-2-yl]-amine [118];
(3-Fluoro-phenyl)-[4-(4-methoxy-phenyl)-pyrimidin-2-yl]-amine [119];
3-[2-(3,5-Dimethoxy-phenylamino)-pyrimidin-4-yl]-phenol [121];
3-[2-(4-Hydroxy-phenylamino)-pyrimidin-4-yl]-phenol [122];
[4-(2,5-Difluoro-phenyl)-pyrimidin-2-yl]-(3-nitro-phenyl)-amine [123];
[4-(4-Methoxy-phenyl)-pyrimidin-2-yl]-(6-methoxy-pyridin-3-yl)-amine [124];
{3-[2-(6-Methoxy-pyridin-3-ylamino)-pyrimidin-4-yl]-phenyl}-methanol [125];
(3-Nitro-phenyl)-{4-[4-(2-[1,2,4]triazol-1-yl-ethyl)-phenyl]-pyrimidin-2-yl}-amine [126];
(1-{4-[2-(3-Nitro-phenylamino)-pyrimidin-4-yl]-benzyl}-piperidin-2-yl)-methanol [127];
N-Methyl-N-{3-[2-(3-nitro-phenylamino)-pyrimidin-4-yl]-phenyl}-methanesulfonamide [129];
N-{3-[2-(3-Hydroxy-phenylamino)-pyrimidin-4-yl]-phenyl}-N-methyl-methanesulfonamide [130];
N-{3-[2-(4-Hydroxy-phenylamino)-pyrimidin-4-yl]-phenyl}-N-methyl-methanesulfonamide [131]; and
N-{3-[2-(6-Methoxy-pyridin-3-ylamino)-pyrimidin-4-yl]-phenyl}-N-methyl-methanesulfonamide [132].

29. A compound according to claim 28 which exhibits an IC₅₀ value for kinase inhibition of less than 10 μM.

30. A compound according to claim 28 which exhibits an IC₅₀ value for kinase inhibition of less than 1 μM.

31. A compound according to claim 28 which exhibits an IC₅₀ value for kinase inhibition of less than 0.1 μM.

32. A compound according to claim 28 which exhibits an IC₅₀ value (average) of less than 10 μM against one or more transformed human cell lines in vitro as measured by a 72-h MTT cytotoxicity assay.

33. A compound according to claim 28 which exhibits an IC₅₀ value (average) of less than 5 μM against one or more transformed human cell lines in vitro as measured by a 72-h MTT cytotoxicity assay.

34. A compound according to claim 28 which exhibits an IC₅₀ value (average) of less than 1 μM against one or more transformed human cell lines in vitro as measured by a 72-h MTT cytotoxicity assay.

35. A pharmaceutical composition comprising a compound according to claim 1 or 28 admixed with a pharmaceutically acceptable diluent, excipient or carrier.

36. A method of treating a proliferative disorder, said method comprising administering to a subject in need thereof, a compound according to claim 1, or a pharmaceutically acceptable salt thereof, in an amount sufficient to treat the proliferative disorder.

37. The method according to claim 36 wherein the proliferative disorder is cancer or leukemia.

38. The method according to claim 36 wherein the proliferative disorder is glomerulonephritis, rheumatoid arthritis, psoriasis or chronic obstructive pulmonary disorder.

39. A method of treating a viral disorder, said method comprising administering to a subject in need thereof, a compound according to claim 1, or a pharmaceutically acceptable salt thereof, in an amount sufficient to treat the viral disorder.

40. The method according to claim 39 wherein the viral disorder is selected from human cytomegalovirus (HCMV), herpes simplex virus type 1 (HSV-1), human immunodeficiency virus type 1 (HIV-1), and varicella zoster virus (VZV).

41. A method of treating a CNS disorder, said method comprising administering to a subject in need thereof, a compound according to claim 1, or a pharmaceutically acceptable salt thereof, in an amount sufficient to treat the CNS disorder.

42. The method according to claim 41 wherein the CNS disorder is Alzheimer's disease or bipolar disorder.

43. A method of treating alopecia, said method comprising administering to a subject in need thereof, a compound according to claim 1, or a pharmaceutically acceptable salt thereof, in an amount sufficient to treat alopecia.

44. A method of treating a stroke, said method comprising administering to a subject in need thereof, a compound according to claim 1, or a pharmaceutically acceptable salt thereof, in an amount sufficient to treat the stroke.

45. The method according to claim 36 wherein the compound is administered in an amount sufficient to inhibit at least one PLK enzyme.

46. The method according to claim 45 wherein the PLK enzyme is PLK1.

47. The method according to claim 36 wherein the compound is administered in an amount sufficient to inhibit at least one CDK enzyme.

48. The method according to claim 47 wherein the CDK enzyme is CDK1, CDK2, CDK3, CDK4, CDK6, CDK7, CDK8 and/or CDK9.

49. The method according to claim 36 wherein the compound is administered in an amount sufficient to inhibit aurora kinase.

50. A method of treating diabetes, said method comprising administering to a subject in need thereof, a compound according to claim 1, or a pharmaceutically acceptable salt thereof, in an amount sufficient to treat diabetes.

51. The method according to claim 50 wherein the diabetes is Type II diabetes.

52. The method according to claim 50 wherein the compound is administered in an amount sufficient to inhibit GSK.

53. The method according to claim 53 wherein the compound is administered in an amount sufficient to inhibit GSK3β.

54. Use of a compound according to claim 1 in an assay for identifying further candidate compounds capable of inhibiting one or more of a cyclin dependent kinase, GSK and a PLK enzyme.

55. Use according to claim 54 wherein said assay is a competitive binding assay.

56. A process for preparing a compound of formula I as defined in claim 1, said process comprising the steps of: 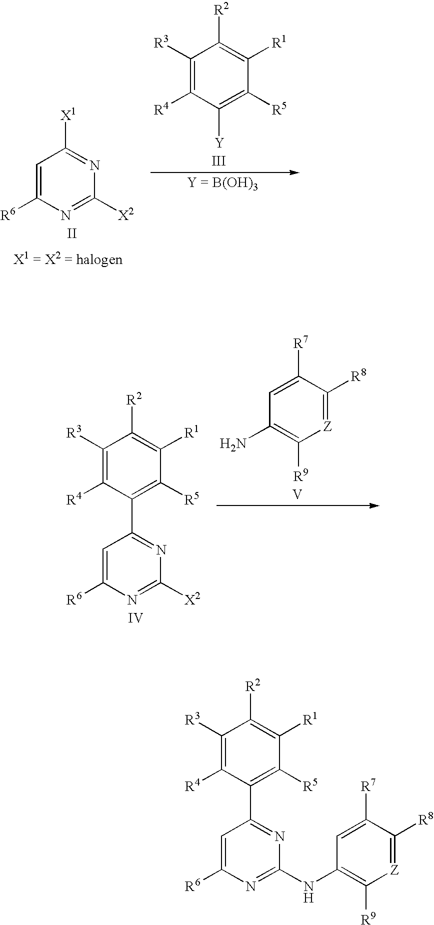
(i) reacting a phenyl boronic acid of formula III with a 2,4-dihalogenated pyrimidine of formula II to form a compound of formula IV; and
(ii) reacting said compound of formula IV with an aniline of formula V to form a compound of formula I.

57. A process for preparing a compound of formula I as defined in claim 1, said process comprising the steps of: 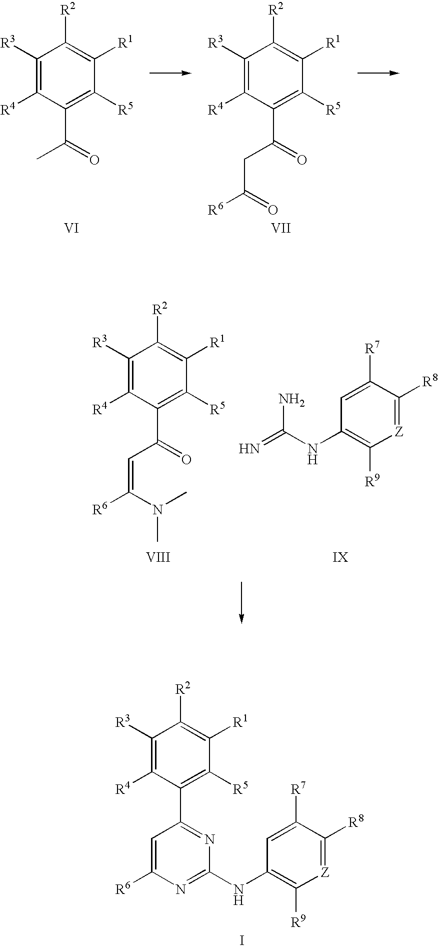
(i) reacting a compound of formula VI with R⁶COCl, where R⁶ is as defined in claim 1, to form a compound of formula VII;
(ii) converting said compound of formula VII to a compound of formula VIII; and
(iii) reacting said compound of formula VIII with a compound of formula IX to form a compound of formula I.

58. A method of treating an aurora kinase-dependent disorder, said method comprising administering to a subject in need thereof, a compound according to claim 1, or a pharmaceutically acceptable salt thereof, in an amount sufficient to inhibit aurora kinase.

59. A method of treating a PLK-dependent disorder, said method comprising administering to a subject in need thereof, a compound according to claim 1, or a pharmaceutically acceptable salt thereof, in an amount sufficient to inhibit PLK.

60. A method of treating an CDK-dependent disorder, said method comprising administering to a subject in need thereof, a compound according to claim 1, or a pharmaceutically acceptable salt thereof, in an amount sufficient to inhibit a cyclin dependent kinase.

61. A method of treating a GSK-dependent disorder, said method comprising administering to a subject in need thereof, a compound according to claim 1, or a pharmaceutically acceptable salt thereof, in an amount sufficient to inhibit GSK.
